(19) United States
(12) Patent Application Publication
(10) Pub. No.: US 2021/0379021 A1
(43) Pub. Date: Dec. 9, 2021

(54) METHOD FOR PREPARING AND DELIVERING BISANTRENE FORMULATIONS

(71) Applicant: Race Oncology Ltd., Welbourne (AU)

(72) Inventors: Daniel E. LEVY, San Mateo, CA (US); John ROTHMAN, Lebanon, NJ (US)

(21) Appl. No.: 17/282,692

(22) PCT Filed: Oct. 4, 2019
(86) PCT No.: PCT/US19/54778

§ 371 Date: Apr. 2, 2021

Related U.S. Application Data
(60) Provisional application No. 62/741,347, filed on Oct. 4, 2018.

(51) Int. Cl. A61K 31/4178; A61K 9/19; A61K 9/16; A61K 9/00; A61K 31/138; A61K 31/454; A61K 31/4196; A61K 31/675; A61K 31/337; A61K 31/519; A61K 31/513; A61K 39/395; A61K 38/20; A61K 33/36; A61K 31/436; A61K 31/5377; A61K 31/4545; A61K 31/517; A61K 31/7068; A61K 31/198

(52) U.S. Cl. CPC A61K 31/4178; A61K 38/2006; A61K 9/1688; A61K 9/0019; A61K 31/138; A61K 31/454; A61K 31/4196; A61K 31/675; A61K 31/337; A61K 31/519; A61K 31/513; A61K 39/3955; A61K 38/2013; A61K 33/36; A61K 31/436; A61K 38/2026; A61K 31/5377; A61K 38/2033; A61K 38/204; A61K 31/4545; A61K 31/517; A61K 31/7068; A61K 31/198; A61K 9/19

(57) ABSTRACT

The present invention is directed to an improved method for preparing bisantrene, specifically bisantrene dihydrochloride, for intravenous administration, as well as to preparations of bisantrene dihydrochloride for intravenous administration. The present invention is also directed to methods for treatment of malignancies treatable by administration of bisantrene, which can include administration of additional anti-neoplastic agents, wherein the bisantrene is prepared by a method according to the present invention.

### CROSS-REFERENCE TO RELATED APPLICATION

This application claims the benefit of U.S. Provisional Patent application Ser. No. 67/741,347 by D. E. Levy et al., entitled “Method for Preparing and Delivering Bisantrene Formulations,” and filed on Oct. 4, 2018, the contents of which are hereby incorporated in their entirety by this reference.

### FIELD OF THE INVENTION

This invention is directed to an improved method for preparing bisantrene for intravenous administration and to preparations of bisantrene for intravenous administration, as well as to methods for treatment of malignancies treatable by administration of bisantrene, which can include administration of additional antineoplastic agents.

### BACKGROUND OF THE INVENTION

The search for and identification of cures for many life-threatening diseases that plague humans still remains an empirical and sometimes serendipitous process. While many advances have been made from basic scientific research to improvements in practical patient management, there still remains tremendous frustration in the rational and successful discovery of useful therapies particularly for life-threatening diseases such as cancer, inflammatory conditions, infection, and other conditions.

Since the “War on Cancer” began in the early 1970's by the United States National Cancer Institute (NCI) of the National Institutes of Health (NIH), a wide variety of strategies and programs have been created and implemented to prevent, diagnose, treat and cure cancer. One of the oldest and arguably most successful programs has been the synthesis and screening of small chemical entities (<1500 MW) for biological activity against cancer. This program was organized to improve and streamline the progression of events from chemical synthesis and biological screening to preclinical studies for the logical progression into human clinical trials with the hope of finding cures for the many types of life-threatening malignant tumors. The synthesis and screening of hundreds of thousands of chemical compounds from academic and industrial sources, in addition to the screening of natural products and extracts from prokaryotes, invertebrate animals, plant collections, and other sources from all over the world has been and continues to be a major approach for the identification of novel lead structures as potential new and useful medicines. This is in addition to other programs including biotherapeutics designed to stimulate the human immune system with vaccines, therapeutic antibodies, cytokines, lymphokines, inhibitors of tumor blood vessel development (angiogenesis) or gene and antisense therapies to alter the genetic makeup of cancer cells, and other biological response modifiers.

The work supported by the NCI, other governmental agencies both domestic and foreign in academic or industrial research and development laboratories has resulted in an extraordinary body of biological, chemical and clinical information. In addition, large chemical libraries have been created, as well as highly characterized in vitro and in vivo biological screening systems that have been successfully used. However, from the tens of billions of dollars spent over the past thirty years supporting these programs both preclinically and clinically, only a small number of compounds have been identified or discovered that have resulted in the successful development of useful therapeutic products. Nevertheless, the biological systems both in vitro and in vivo and the “decision trees” used to warrant further animal studies leading to clinical studies have been validated. These programs, biological models, clinical trial protocols, and other information developed by this work remain critical for the discovery and development of any new therapeutic agent.

Unfortunately, many of the compounds that have successfully met the preclinical testing and federal regulatory requirements for clinical evaluation were either unsuccessful or disappointing in human clinical trials. Many compounds were found to have untoward or idiosyncratic side-effects that were discovered during human clinical Phase I dose-escalation studies used to determine the maximum tolerated dose (MTD) and side-effect profile. In some cases, these toxicities or the magnitude of their toxicity were not identified or predicted in preclinical toxicology studies. In other cases, chemical agents where in vitro and in vivo studies suggested a potentially unique activity against a particular tumor type, molecular target or biological pathway were not successful in human Phase II clinical trials where specific examination of particular cancer indications/types were evaluated in government sanctioned (e.g., U.S. FDA), IRB approved clinical trials. In addition, there are those cases where potential new agents were evaluated in randomized Phase III clinical trials where a significant clinical benefit could not be demonstrated; such cases have also been the cause of great frustration and disappointment. Finally, a number of compounds have reached commercialization, but their ultimate clinical utility has been limited by poor efficacy as monotherapy (<25% response rates) and untoward dose-limiting side-effects (Grade III and IV) (e.g., myelosuppression, neurotoxicity, cardiotoxicity, gastrointestinal toxicities, or other significant side effects).

In many cases, after the great time and expense of developing and moving an investigational compound into human clinical trials and where clinical failure has occurred, the tendency has been to return to the laboratory to create a better analog, look for agents with different structures but potentially related mechanisms of action, or try other modifications of the drug. In some cases, efforts have been made to try additional Phase I or II clinical trials in an attempt to make some improvement with the side-effect profile or therapeutic effect in selected patients or cancer indications. In many of those cases, the results did not realize a significant enough improvement to warrant further clinical development toward product registration. Even for commercialized products, their ultimate use is still limited by suboptimal performance.

With so few therapeutics approved for cancer patients and the realization that cancer is a collection of diseases with a multitude of etiologies and that a patient's response and survival from therapeutic intervention is complex with many factors playing a role in the success or failure of treatment including disease indication, stage of invasion and metastatic spread, patient gender, age, health conditions, previous therapies or other illnesses, genetic markers that can either promote or retard therapeutic efficacy, and other factors, the opportunity for cures in the near term remains elusive. Moreover, the incidence of cancer continues to rise due to a number of risk factors, such as, but not limited to, smoking and diet. In addition, with advances in diagnosis such as mammography for breast cancer and PSA tests for prostate cancer, more patients are being diagnosed at a younger age. For difficult to treat cancers, a patient's treatment options are often exhausted quickly resulting in a desperate need for additional treatment regimens. Even for the most limited of patient populations, any additional treatment opportunities would be of considerable value. This invention focuses on inventive compositions and methods for improving the therapeutic benefit of suboptimally administered chemical compounds including bisantrene and derivatives and analogs thereof, in particular by the use of improved preparations of bisantrene to improve stability and bioavailability.

Relevant literature includes Foye, W. O., “Cancer Chemotherapeutic Agents,” American Chemical Society, 1995, and Dorr, R. T., and Von Hoff, D. D., “Cancer Chemotherapy Handbook,” Appleton and Lange, 1994.

Bisantrene, generally employed as the dihydrochloride, is an unusual agent with direct cytotoxic action as well as genomic and immunologic methods of action. The chemical name for bisantrene dihydrochloride is 9,10-anthracenedicarboxaldehyde-bis [(4, 5-dihydro-1H-imidazole-2-yl) hydrazine] dihydrochloride. Although it is structurally an anthracene, it is classed as an anthracycline chemotherapeutic agent due to its mechanism of action and therapeutic activities. These are drugs with planar structures based around a resonant aromatic ring structure that intercalates within the helices of DNA and disrupt various functions, including replication, presumably due to a strong inhibitory effect on the enzyme topoisomerase II. It was found that, like other anthracyclines, it could kill tumor cells in clonogenic assays and intercalate with DNA, where it inhibits both DNA and RNA synthesis. The primary chemotherapeutic mechanism for bisantrene is its preferential binding to A-T rich regions where it effects changes to supercoiling and initiates strand breaks in association with DNA associated proteins. This results from the inhibition of the enzyme topoisomerase II, which relaxes DNA coiling during replication. It was found that while inactive orally, it was effective intravenously (i.v.), intraperitoneally (i.p.), or subcutaneously (s.c.) in cancer models using colon 26, Lewis lung, Ridgway osteosarcoma, B16, Lieberman plasma cell, P388 or L1210 cancer cells. Activity in clonogenic assays from 684 patients was seen in breast, small cell lung, large cell lung, squamous cell lung, ovarian, pancreatic, renal, adrenal, head and neck, sarcoma, gastric, lymphoma and melanoma tumor cells, but not in colorectal cancer. Importantly, a lack of cross resistance with Adriamycin and mitoxantrone was found.

However, bisantrene dihydrochloride has a number of toxicities. Toxicity studies in dogs and monkeys revealed that at high doses leukopenia, anorexia, diarrhea, injection site necrosis, enterocolitis, muscle degeneration, and pulmonary edema were observed. Although anthracyclines have limited therapeutic utility due to their propensity to cause cardiac toxicity, this primary dose-limiting toxicity characteristic of the anthracycline class of drugs was observed to be less for bisantrene than that of any other agent in the anthracycline class.

Because of its lack of aqueous solubility at physiologic pH, bisantrene precipitates in the body have been observed in studies of rabbits and calves. Deposition of drug into the tissues has been associated with phlebitis. Its lack of aqueous solubility has limited its bioavailability.

Bisantrene is normally administered intravenously. However, the intravenous administration of bisantrene has been associated with severe local venous toxicity. Various alternatives have been tried to minimize this toxicity. In one alternative, bisantrene doses have been infused via central venous access devices over 1 hour. In another alternative, bisantrene has been infused through peripheral veins over 2 hours, and has been “piggybacked” into a running dextrose infusion in an attempt to lessen delayed swelling in the arm used for infusion. In yet another alternative, to reduce venous irritation, hyperpigmentation, drug extravasation, and anaphylactoid reactions, patients have been given hydrocortisone (50 mg i.v.) and the antihistamine diphenhydramine (50 mg i.m.) immediately prior to bisantrene. Resultant nausea is frequently controlled with anti-emetic agents.

However, there is a need for improved formulations of bisantrene that reduce toxicity, improve bioavailability, and prevent venous damage, extravasation of the drug, and phlebitis. There is also a need for improved methods of preparation of such formulations, as well as a need for improved methods of administration of such formulations.

### SUMMARY OF THE INVENTION

The present invention is directed to improved formulations of bisantrene, particularly bisantrene dihydrochloride, that reduce toxicity, improved bioavailability, and prevent venous damage, extravasation of the drug, phlebitis, and other significant side effects by removing particulate contaminants from the formulations, as well as methods for preparation of the formulations. The present invention is also directed to methods for administration of the improved formulations to treat diseases and conditions treatable by administration of bisantrene, particularly malignancies.

One aspect of the invention is a method for preparing bisantrene dihydrochloride units for delivery to a patient in need of treatment with bisantrene dihydrochloride comprising the steps of:

(1) preparing an initial stock solution of bisantrene dihydrochloride;

(2) filtering the initial stock solution of bisantrene dihydrochloride;

(3) aliquoting the initial stock solution of bisantrene dihydrochloride into vials; and

(4) lyophilizing the aliquoted stock solution in the vials.

Typically, the initial stock solution of bisantrene dihydrochloride is prepared in sterile water for injection. Typically, the initial stock solution is prepared at a temperature of about 20° C. to about 25° C. Alternatively, the initial stock solution is prepared at a temperature of about 4° C. The initial mixture of bisantrene dihydrochloride can be at a concentration of about 40 mg/mL, at about 25 mg/mL, or at an intermediate concentration of any value between about 25 mg/mL and about 40 mg/m L.

Typically, the initial stock solution is filtered through 1 to 3 filters.

When the initial stock solution is filtered through one filter, typically, the filter has a filtration cutoff of about 0.2 μm. When the initial stock solution is filtered through two filters, typically, the first filter has a filtration cutoff of about 1-2 μm and the second filter has a filtration cutoff of about 0.2 μm. When the initial stock solution is filtered through three filters, typically, the first filter has a filtration cutoff of about 4-6 μm, the second filter has a filtration cutoff of about 1-2 μm, and the third filter has a filtration cutoff of about 0.2 μm.

The vials can be plastic vials or glass vials. When glass vials are used, they are typically silanized; typically, the silanization is performed by coating the interior of the vials with an organofunctional alkoxysilane selected from the group consisting of (3-aminopropyl)-triethoxysilane, (3-aminopropyl)-diethoxymethylsilane, (3-aminopropyl)-dimethyl-ethoxysilane, (3-aminopropyl)-trimethoxysilane, (3-glycidoxypropyl)-dimethyl-ethoxysilane, (3-mercaptopropyl)-trimethoxysilane, (3-mercaptopropyl)-methyl dimethoxysilane, and derivatives thereof. When plastic vials are used, the plastic is typically selected from the group consisting of cyclic olefin polymer (COP) plastic, cyclic olefin copolymer (COC) plastic, high-density polyethylene plastic, and high-density non-nucleated polypropylene plastic.

Typically, the volume of stock solution aliquoted into each vial is consistent with delivery of about 295 mg of bisantrene dihydrochloride into each vial. Typically, the volume of stock solution aliquoted into each vial is from about 5.0 mL to about 7.5 mL based on the concentration of the initial stock solution. Preferably, the volume of stock solution aliquoted into each vial is from about 5.625 mL to about 6.875 mL based on the concentration of the initial stock solution. Typically, the vials are of a volume from about 8 mL to about 12 mL in volume. Preferably, the vials are of a volume from about 9 mL to about 11 mL in volume. More preferably, the vials are about 10 mL in volume.

Another aspect of the present invention is a method for delivering bisantrene dihydrochloride units to a patient in need of treatment with bisantrene dihydrochloride comprising the steps of:

(1) reconstituting the contents of a bisantrene dihydrochloride unit vial with sterile water;

(2) filtering the reconstituted bisantrene dihydrochloride into a suitable i.v. infusion vehicle; and

(3) infusing into a patient a therapeutic volume of the bisantrene dihydrochloride-infusion vehicle formulation.

Typically, the bisantrene dihydrochloride units comprise about 295 mg of lyophilized bisantrene dihydrochloride. Typically, the contents of a bisantrene dihydrochloride unit vial are reconstituted with about 9 mL to about 11 mL of sterile water; preferably, the contents of a bisantrene dihydrochloride unit vial are reconstituted with about 10 mL of sterile water.

In one alternative, the filter is a sterile syringe filter. Typically, the sterile syringe filter has a filtration cutoff in a range of from about 0.15 μm to about 0.25 μm. Preferably, the sterile syringe filter has a filtration cutoff in a range of from about 0.175 μm to about 0.225 μm. More preferably, the sterile syringe filter has a filtration cutoff of about 0.2 μm.

Typically, the suitable i.v. infusion vehicle is 5% dextrose in water.

Typically, a volume of the i.v. infusion vehicle equivalent to the volume of reconstituted bisantrene dihydrochloride and any filter wash volume is removed before filtration of the reconstituted bisantrene dihydrochloride into the i.v. infusion vehicle.

Typically, the volume of the i.v. infusion vehicle is selected from the group consisting of 500 mL and 1 L. When the volume of the i.v. infusion vehicle is 500 mL, typically a single vial of lyophilized bisantrene dihydrochloride is reconstituted and filtered into the i.v. infusion vehicle. When the volume of the i.v. infusion vehicle is 1 L, typically two vials of lyophilized bisantrene dihydrochloride is reconstituted and filtered into the i.v. infusion vehicle.

In another alternative, the bisantrene dihydrochloride-infusion vehicle formulation is infused into a patient through an i.v. infusion set containing an in-line filter. Typically, the in-line filter has a filtration cutoff in a range of from about 0.15 μm to about 0.25 μm. Preferably, the in-line filter has a filtration cutoff in a range of from about 0.175 μm to about 0.225 μm. More preferably, the in-line filter has a filtration cutoff of about 0.2 μm.

Typically, the duration of the infusion is from about 1.5 hours to about 2.5 hours. Preferably, the duration of the infusion is from about 1.75 hours to about 2.25 hours. More preferably, the duration of the infusion is about 2.0 hours.

Typically, the dosage received by the patient is from about 200 mg/m² to about 300 mg/m² body surface area. Preferably, the dosage received by the patient is from about 225 mg/m² to about 275 mg/m² body surface area. More preferably, the dosage received by the patient is about 250 mg/m² body surface area.

In one alternative, the method can further comprise the step of administering to the patient a therapeutically effective quantity of an additional therapeutic agent.

In one alternative, the bisantrene dihydrochloride is administered to the patient to treat a malignancy selected from the group consisting of: breast cancer, acute myelocytic leukemia, acute lymphocytic leukemia of childhood, myelodysplastic syndrome, chronic myelocytic leukemia, chronic lymphocytic leukemia, Hodgkin's lymphoma, non-Hodgkin's lymphoma, mycosis fungoides, prostate cancer, lung small-cell carcinoma, lung non-small cell carcinoma, glioblastoma, a malignancy characterized by overexpressed topoisomerase II, a malignancy characterized by overexpressed and/or mutated EGFR, ovarian cancer, renal cancer, melanoma, gastric cancer, adrenal cancer, head and neck cancer, hepatocellular cancer, hypernephroma, bladder cancer, myeloma, and localized polyp stage colon cancer. When the method further comprises the step of administering to the patient an additional therapeutic agent, suitable additional therapeutic agents for treatment of these malignancies are described. Other additional therapeutic agents can be used. However, generally, in the absence of compatibility studies, administration of bisantrene dihydrochloride is recommended as a single drug and must not be mixed with other products, including additional therapeutic agents. Therefore, when one or more additional agents are administered, the one or more additional agents are administered separately from the bisantrene dihydrochloride, such as in one or more pharmaceutical compositions.

In yet another alternative, the bisantrene dihydrochloride is administered with a therapeutically effective quantity of an additional agent selected from the group consisting of: an agent inducing immunoactivity; an agent inducing macrophage activation; a cytokine; an agent inhibiting telomerase; an agent inhibiting survivin; an agent inhibiting methylation or modulating demethylation; an adjuvant; an antibody; an innate or adaptive immune stimulator; a checkpoint inhibitor; a mTOR antagonist; an Akt inhibitor; a notch inhibitor; an Hsp90 inhibitor; a phosphatidylinositide 3-kinase inhibitor; a kinase inhibitor; taxane; and taxol.

In yet another alternative, the bisantrene dihydrochloride is administered together with a therapeutically effective quantity of an additional agent, wherein the additional agent is a pyrimidine analog antimetabolite.

### DETAILED DESCRIPTION OF THE INVENTION

Bisantrene has been known for many years and was never fully developed for oncology in the United States. Phlebitis was observed during i.v. administration and the occurrence of phlebitis resulted in the need to deliver the bisantrene dihydrochloride through a central venous line.

The structure of bisantrene dihydrochloride is shown in Formula (I), below.

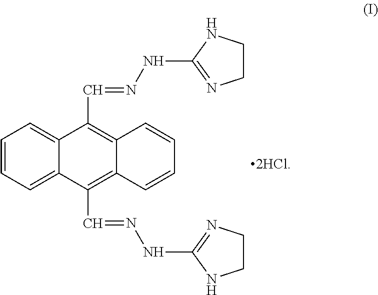

Bisantrene, more specifically bisantrene dihydrochloride, is a tricyclic aromatic compound with the chemical name, 9,10-anthracenedicarboxaldehyde bis[(4,5-dihydro-1H-imidazol-2-Ohydrazine] dihydrochloride. The molecular formula is C₂₂H₂₂N₈.2HCl and the molecular weight, 471.4. The alkylimidazole side chains are very basic and, at physiological pH, are positively charged. This is believed to facilitate electrostatic attractions to negatively charged ribose phosphate groups in DNA.

Bisantrene has shown antitumor activity in murine tumor models including P-388 leukemia and B-16 melanoma (R. V. Citarella et al., “Anti-Tumor Activity of 9,10-Anthracenedicarboxaldehyde bis[(4,5-dihydro-1H-imidazol-2-yl)hydrazine]dihydrochloride (Abstract #23) in Abstracts of the 20^{th} Interscience Conference on Antimicrobial Agents and Chemotherapy (Bethesda, Md., American Society for Microbiology 1980)). Human tumor cells that were sensitive to bisantrene as assessed by in vitro colony-forming assays include breast cancer, ovarian cancer, renal cancer, small cell and non-small cell lung cancer, lymphoma, acute myelogenous leukemia, melanoma, gastric cancer, adrenal cancer, and head and neck cancer (D. D. Von Hoff et al, “Activity of 9,10-Anthracenedicarboxaldehyde bis[(4,5-dihydro-1H-imidazol-2-yl)hydrazine]dihydrochloride (CL216,942) in a Human Tumor Cloning System,” Cancer Chemother. Pharmacol. 6: 141-144 (1981) (“Von Hoff et al. (1981a)”). In phase I clinical trials bisantrene showed activity in hepatocellular cancer and hypernephroma (one patient each) (D. D. Von Hoff et al., “Phase I Clinical Investigation of 9,10-Anthracenedicarboxaldehyde bis[(4,5-dihydro-1H-imidazol-2-yl)hydrazine]dihydrochloride (CL216,942),” Cancer Res. 3118-3121 (1981) (“Von Hoff et al. (1981b)”) and in lymphoma, myeloma, melanoma, renal cancer, and tumors of the bladder and lung (D. S. Alberts et al., “Phase I Clinical Investigation of 9,10-Anthracenedicarboxaldehyde bis[(4,5-dihydro-1H-imidazol-2-yl)hydrazine] Dihydrochloride with Correlative in Vitro Human Tumor Clonogenic Assay,” Cancer Res. 42: 1170-1175 (1982)). Phase I activity was also observed in two other hypernephroma patients (R. J. Spiegel et al., “Phase I Clinical Trial of 9,10-Anthracene Dicarboxaldehyde (Bisantrene) Administered in a Five-Day Schedule,” “Cancer Res. 42: 354-358 (1982)). Bisantrene was inactive in human colon cancer tested in vitro or in vivo (M. C. Perry et al. “Phase II Trial of Bisantrene in Advanced Colorectal Cancer: A Cancer and Leukemia Group B Study,” Cancer Treat. Rep. 66: 1997-1998 (1982); Von Hoff et al. (1981a); Von Hoff et al. (1981b). It was also inactive in refractory malignant melanoma (D. S. Alberts et al., “Phase II Evaluation of Bisantrene Hydrochloride in Refractory Malignant Melanoma,” Invest. New Drugs 5: 289-292 (1987)).

In Phase II clinical trials, bisantrene was active in patients with metastatic breast cancer (H.-Y. Yap et al., “Bisantrene, an Active New Drug in the Treatment of Metastatic Breast Cancer,” Cancer Res. 43: 1402-1404 (1983)). Partial response rates were observed in heavily pretreated patients with metastatic breast cancer. However, the study was terminated because of significant local toxicity observed.

The mechanism of action for bisantrene has been studied. Bisantrene has been shown to induce altered DNA supercoiling indicative of DNA intercalation (G. T. Bowden et al., “Comparative Molecular Pharmacology in Leukemic L1210 cells of the Anthracene Anticancer Drugs Mitoxantrone and Bisantrene, Cancer Res. 45: 4915-4920 (1985)). In L-1210 leukemia cells bisantrene was also shown to induce protein-associated DNA strand breaks typical of drug-induced inhibition of DNA topoisomerase II enzymes (Bowden et al., 1985). Both cytotoxicity and the DNA strand breaks appear to be reduced in hypoxic conditions (C. U. Ludwig et al., “Reduced Bisantrene-Induced Cytotoxicity and Protein-Associated DNA Strand Breaks Under Hypoxic Condition,” Cancer Treat. Rep. 68: 367-372 (1984)). The noncovalent binding of bisantrene to DNA appears to comprise two types of interactions: (1) intercalation of the planar anthracene moiety between DNA base pairs, and (2) electrostatic binding between negatively charged ribose phosphates of DNA and positively charged basic nitrogens on the alkyl side chains of the drug. This is reflected in the biphasic DNA dissociation curves for bisantrene in calf thymus DNA in vitro (W. O. Foye et al., “DNA-Binding Abilities of Bisguanylhydrazones of Anthracene-9,10-dicarboxaldehyde,” Anti-Cancer Drug Design 1: 65-71 (1986)).

In one alternative, bisantrene vials have been reconstituted with 2 to 5 mL of Sterile Water for Injection, USP, and then diluted with approximately 0.1 to 0.5 mg/mL in D5W (5% dextrose in water). Bisantrene is incompatible with saline and unstable in light (G. Powis et al., “Pharmacokinetic Study of ADAH in Humans and Sensitivity of ADAH to Light” (Abstract #C-74),” ASCO Proc. 1: 19 (1982).

Maximally tolerated doses in several bisantrene phase I schedules include: (1) 200 mg/m² weekly×3 (150 mg/mg² for patients with poor bone marrow reserve (e.g., those patients who have received radiotherapy or extensive chemotherapy regimens) (Alberts et al. (1982), supra); (2) 150 mg/m² weekly×3 (repeat every 4-5 week) (B.-S. Yap et al., “Phase I Clinical Evaluation of 9,10-Anthracenedicarboxaldehyde[bis(4,5-dihydro-1H-imidazol-2-yl)hydrazone]dihydrochloride (Bisantrene),” Cancer Treat. Rep. 66: 1517-1520 (1982)) (3) 260 mg/m² monthly (every 3-4 week) (240 mg/mg² for patients with poor bone marrow reserve (e.g., those patients who have received radiotherapy or extensive chemotherapy regimens) (Von Hoff et al., 1981b); and (4) 80 mg/m² daily×5 (repeat every 4 week) (R. J. Spiegel et al. (1982), supra).

More than 95% of bisantrene is bound to plasma proteins and the drug has a long terminal plasma half-life. There appeared to be three phases of elimination: an initial distributive phase of 6 minutes, a beta phase of approximately 1.5 hours, and a final gamma elimination phase of 23 to 54 hours (Alberts et al. (1983), supra). Typical areas under the plasma concentration×time curve are 4.4 to 5.7 mg-h/mL following intravenous doses of 260 to 340 mg/m², respectively (Alberts et al. 1983, supra). Less than 7% of a bisantrene dose is excreted in the urine and the majority of the drug is eliminated by the hepatobiliary route. The drug may be metabolized to some extent in vivo. In vitro bisantrene is a substrate for hepatic microsomal enzymes but specific metabolites have not been identified. Preclinical drug distribution studies showed that the tissues with the highest concentration (in descending order) are kidney, liver, gallbladder, spleen, lung, and heart. Brain levels were extremely low. The drug did distribute to lymph nodes and bone marrow (W. H. Wu & G. Nicolau, “Disposition and Metabolic Profile of a New Antitumor Agent, CL 216,942 (Bisantrene) in Laboratory Animals,” Cancer Treat Rep. 66: 1173-1185 (1982)).

The major dose-limiting toxic effect of bisantrene is leukopenia (Von Hoff et al. 1981b; Alberts et al. 1982, supra; Spiegel et al. 1982, supra; Yap et al 1982, supra)). On a schedule of every 3 to 4 weeks, the nadir for myelosuppression was 9 days with recovery by 19 days (Von Hoff et al. 1981b). Thrombocytopenia was mild although bisantrene can also inhibit platelet aggregation (M. E. Rybak et al., “The Effects of Bisantrene on Human Platelets,” Invest. New Drugs 4: 119-125 (1986)). Anemia and cumulative myelosuppressive toxic effects were not encountered with this drug.

In addition to myelosuppression, bisantrene produced severe phlebitis along peripheral veins used for drug infusion (Von Hoff et al. 1981b; Alberts et al. 1982). This results from drug precipitation in veins which has been documented in experimental models (G. Powis & J. S. Kovach 1983). The drug is a potent vesicant and produces severe local tissue necrosis if inadvertently extravasated (Von Hoff et al 1981b). Severe arm swelling, hyperpigmented veins, and punctate perivenous orange discolorations have been occasionally observed following bisantrene infusions given through peripheral veins. The arm swelling appeared to be the result of a localized capillary leak syndrome in the arm used for infusion. In an experimental mouse skin model, extravasation necrosis was blocked with a local injection of sodium bicarbonate which physically decomposes bisantrene (R. T. Dorr et al., “Bisantrene Solubility and Skin Toxicity Studies: Effect of Sodium Bicarbonate as a Local Ulceration Antidote,” Invest. New Drugs 2: 351-357 (1984)).

Up to 10% of patients experienced anaphylactoid reactions following a bisantrene infusion (J. W. Myers et al., “Anaphylactoid Reactions Associated with Bisantrene Infusions,” Invest. New Drugs 1: 85-88 (1983)). Symptoms included chills, chest pain, shortness of breath, flushing, and pruritus. These effects may be caused by drug-induced histamine release. Hypotension is also reported with bisantrene, and prolongation of the infusion was recommended to reduce this complication (Von Hoff et al., 1981b). In addition, a few patients experienced diaphoresis and palpitations, usually near the end of a bisantrene infusion (Von Hoff et al., 1981b). The drug was not cardiotoxic in animals and use in the clinic has confirmed less cardiotoxicity than other agents in its class. No patients experienced electrocardiographic changes while receiving the drug and radioangiocardiographic monitoring demonstrated no decrease in ejection fraction or any other significant change in cardiac function (J. W. Myers et al., “Radioangiocardiographic Monitoring in Patients Receiving Bisantrene,” Am. J. Clin. Oncol. 7: 129-130 (1984)).

Bisantrene has been reported to produce very little nausea or vomiting. Alopecia (hair loss) is also less intense with bisantrene compared with doxorubicin (J. D. Cowan et al., “Randomized Trial of Doxorubicin, Bisantrene, and Mitoxantrone in Advanced Breast Cancer: A Southwest Oncology Group Study,” J. Nat'l Cancer Inst. 83: 1077-1084 (1991)). However, bisantrene can produce a mild fever in some patients and malaise may be particularly common. This was reported by up to one-half of patients studied (Yap et al. (1982), supra).

Various formulations suitable for use in the administration of bisantrene or derivatives or analogs thereof are known in the art. U.S. Pat. No. 4,784,845 to Desai et al. discloses a composition of matter for delivery of a hydrophobic drug (i.e., bisantrene or a derivative or analog thereof) comprising: (i) the hydrophobic drug; (ii) an oleaginous vehicle or oil phase that is substantially free of butylated hydroxyanisole (BHA) or butylated hydroxytoluene (BHT); (iii) a co-surfactant or emulsifier; (iv) a co-surfactant or auxiliary emulsifier; and (v) benzyl alcohol as a co-solvent. U.S. Pat. No. 4,816,247 by Desai et al. discloses a composition of matter for delivery by intravenous, intramuscular, or intraarticular routes of hydrophobic drugs (such as bisantrene or a derivative or analog thereof) comprising: (i) the hydrophobic drug; (ii) a pharmaceutically acceptable oleaginous vehicle or oil selected from the group consisting of: (a) naturally occurring vegetable oils and (b) semisynthetic mono-, di-, and triglycerides, wherein the oleaginous vehicle or oil is free of BHT or BHA; (iii) a surfactant or emulsifier; (iv) a co-surfactant or emulsifier; (v) an ion-pair former selected from C₆-C₂₀ saturated or unsaturated aliphatic acids when the hydrophobic drug is basic and a pharmaceutically acceptable aromatic amine when the hydrophobic drug is acidic; and (vi) water. U.S. Pat. No. 5,000,886 to Lawter et al. and U.S. Pat. No. 5,143,661 to Lawter et al. disclose compositions for delivery of pharmaceutical agents such as bisantrene or a derivative or analog thereof comprising a microcapsule, wherein the microcapsule includes a hardening agent that is a volatile silicone fluid. U.S. Pat. No. 5,070,082 to Murdock et al., U.S. Pat. No. 5,077,282 to Murdock et al., and U.S. Pat. No. 5,077,283 to Murdock et al. disclose prodrug forms of poorly soluble hydrophobic drugs, including bisantrene and derivatives and analogs, that are salts of a phosphoramidic acid. U.S. Pat. No. 5,116,827 to Murdock et al. and U.S. Pat. No. 5,212,291 to Murdock et al. disclose prodrug forms of poorly soluble hydrophobic drugs, including bisantrene and derivatives and analogs, that are quinolinecarboxylic acid derivatives. U.S. Pat. No. 5,378,456 to Tsou discloses compositions containing an anthracene antitumor agent, such as bisantrene or a derivative or analog thereof, in which the bisantrene or derivative or analog thereof is conjugated to or admixed with a divinyl ether-maleic acid (MVE) copolymer. U.S. Pat. No. 5,609,867 to Tsou discloses polymeric 1,4-bis derivatives of bisantrene and copolymers of bisantrene and another monomer, such as a dianhydride.

In general, bisantrene should not be reconstituted in Ringer's solution or other solutions for parenteral use other than water for injection. For infusion, only the 5% dextrose solution should be used. In the absence of compatibility studies, administration of bisantrene is recommended as a single drug and bisantrene must not be mixed with other products. As detailed below, therefore, when one or more additional agents are administered besides bisantrene dihydrochloride, the one or more additional agents are administered separately from the bisantrene dihydrochloride, such as in one or more pharmaceutical compositions.

The present application, therefore, provides improved methods for the preparation and administration of particulate-free bisantrene dihydrochloride, particularly intravenous administration, to treat malignancies and other conditions as described below. As detailed further below, methods according to the present invention can also be applied to derivatives, analogs, and prodrugs of bisantrene dihydrochloride.

Bisantrene dihydrochloride powder is produced by combining solid bisantrene dihydrochloride with sterile water for injection at a concentration of 40 mg/m L. The resulting heterogeneous mixture is filtered first through a 5-μm first filter, then through a 1.2-μm second filter, and finally through an 0.2-μm third filter in order to produce a 40 mg/mL bisantrene solution. Removal of particulates at this stage does not diminish the bisantrene dihydrochloride content in solution, possibly because the insoluble material is a poorly soluble form of bisantrene or bisantrene dihydrochloride that is removed in HPLC prefilters when the bisantrene content is analyzed by HPLC, and therefore never assayed either before or after filtration. The resulting particle-free bisantrene dihydrochloride solution (6.25 mL) is filled into 10-mL vials and lyophilized. Finished vials are sealed under nitrogen and partial vacuum for storage. Storage of the vials is typically at 18° C. to 25° C.

Bisantrene dihydrochloride lyophilized powder, when reconstituted, contains particulates. Although applicants do not intend to be bound by this hypothesis, it is likely that the particulates are microcrystalline forms with limited dissolution rates. The source of these particles may be the freezing step of the lyophilization process. During the freezing step, low temperature induced crystallization may be occurring and may be in concert with nucleation sites on the surfaces of the manufacturing equipment and/or vials.

When bisantrene dihydrochloride is reconstituted and injected into an i.v. bag, analytical results have demonstrated the following. (1) The assay of fully diluted bisantrene dihydrochloride in the i.v. bag was approximately 5% lower than the assay of the reconstituted bisantrene dihydrochloride in the finished vial when samples were collected in glass vials; however, when samples were collected in plastic vials, there was no reduction in bisantrene dihydrochloride concentration. (2) The assay of fully diluted bisantrene dihydrochloride in the i.v. bag when passed through an 0.2-μm filter was approximately 5% lower than the i.v. bag assay when sample was collected in glass vials for analysis. (3) The assay of fully diluted bisantrene dihydrochloride in the i.v. bag when passed through an 0.2-μm filter approximately matched the i.v. bag assay when sample was collected in plastic vials (rather than glass vials). (4) These results suggest that bisantrene or bisantrene dihydrochloride adheres to glass.

Reconstituted bisantrene dihydrochloride formulations can be cleared of particulates by initial filtration of reconstituted bisantrene dihydrochloride through an 0.2-μrn syringe filter while injecting the formulation into an i.v. infusion vehicle for administration to a patient. Additional safety regarding particulates is achieved using an i.v. infusion set equipped with an in-line 0.2-μm filter. Without pre-filtration, reconstituted and diluted bisantrene dihydrochloride formulations will have the tendency to clog 0.2-μm i.v. infusion filters.

In one alternative, a filtration process is used to prepare the drug product. The contents of a reconstituted vial of lyophilized bisantrene dihydrochloride are drawn up and injected into an i.v. bag. An in-line filter is then placed in the infusion line. Typically, the filter placed in the infusion line is an 0.2-μm filter, although, as described below, a filter with a different filtration cutoff can be used. In another alternative, a syringe filter can also be used to perform initial filtration of the reconstituted bisantrene dihydrochloride while injecting the formulation into the i.v. bag; when used, the syringe filter is typically also an 0.2-μm filter, although a syringe filter with a different filtration cutoff can also be used. When used, the use of the syringe filter precedes the in-line filter.

Pre-filtration of stock bisantrene dihydrochloride solution eliminates particulates prior to the lyophilization process. Preparation of a bisantrene dihydrochloride stock solution at room temperature eliminates temperature-induced degradation of the bisantrene dihydrochloride (the active pharmaceutical ingredient (API). Alternatives for preparation of the bisantrene dihydrochloride lyophilized powder include: (i) preparation in plastic vials; (ii) preparation in glass vials; (iii) preparation at about 25 mg/mL; or (iv) preparation at 40 about mg/mL. Alternatively, as described below, preparation can be done at any concentration from about 25 mg/mL to about 40 mg/mL, including, but not limited to, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, or any value between these values. Preparation can be done in plastic vials or glass vials; in most cases, preparation in plastic vials is preferred to avoid nucleation that may occur in glass vials at certain stages of preparation. When preparation is done in plastic vials, the plastic can be selected from the group consisting of cyclic olefin polymer (COP) plastic, cyclic olefin copolymer (COC) plastic, high-density polyethylene plastic, and high-density non-nucleated polypropylene plastic. When preparation is done in glass vials, the glass vials can be coated with a silicone coating, such as an organofunctional alkoxysilane selected from the group consisting of (3-aminopropyl)-triethoxysilane, (3-aminopropyl)-diethoxymethylsilane, (3-aminopropyl)-dimethyl-ethoxysilane, (3-aminopropyl)-trimethoxysilane, (3-glycidoxypropyl)-dimethyl-ethoxysilane, (3-mercaptopropyl)-trimethoxysilane, (3-mercaptopropyl)-methyl-dimethoxysilane, and derivatives thereof. In general, preparation in plastic vials is preferred to avoid possible nucleation occurring on the surface of glass vials.

Elimination of exposure of patients receiving infusions of bisantrene dihydrochloride to particulates reduces or eliminates phlebitis at the site of injection, and also reduces the risk of other side effects, such as venous irritation, hyperpigmentation, drug extravasation, or anaphylactoid reactions. Elimination of exposure of exposure of patients receiving infusions of bisantrene dihydrochloride to particulates allows the use of standard i.v. infusion and eliminates the need for central line infusion. Additionally, elimination of exposure of exposure of patients receiving infusions of bisantrene dihydrochloride to particulates increases the safety of administration of bisantrene dihydrochloride as a chemotherapeutic agent while not reducing its effectiveness. Minimizing or eliminating phlebitis at i.v. infusion sites makes bisantrene safer, more acceptable to patients, and reduces treatment costs. Similarly, eliminating the need for central line infusion makes bisantrene safer, more acceptable to patients, and reduces treatment costs.

One alternative for preparation and administration of bisantrene dihydrochloride is as follows. Bisantrene dihydrochloride is prepared as a lyophilized powder in units of 250 mg bisantrene base (equivalent to 295 mg bisantrene dihydrochloride) in 10-mL vials, sealed under nitrogen and partial vacuum. Although photoprotective vials, such as amber-colored vials, can be used, their use is not essential as the lyophilizer is shielded from light and the finished vials can be packaged in light-protective cardboard boxes or other light-protective packaging. A method of preparation of dosage units includes the following steps. A 40 mg/mL initial mixture of bisantrene dihydrochloride in sterile water for injection is prepared at room temperature. The initial mixture is filtered through a 5-μm filter and then again through a 1.2-μm filter. The filtrate is then filtered again through an 0.2-μm filter to produce a stock solution. The stock solution is then assayed, such as by HPLC; general techniques for HPLC are described in L. R. Snyder et al., “Introduction to Modern Liquid Chromatography” (3^{rd} ed., John Wiley & Sons, New York, 2009). Aliquots of 6.25 mL of a 40 mg/mL stock solution of bisantrene dihydrochloride are filled into 10-mL vials. The bisantrene dihydrochloride in the 10-mL vials are lyophilized to a dry cake. The vials are then sealed under nitrogen and partial vacuum.

Subsequently, for administration of the bisantrene dihydrochloride, the vial contents are reconstituted using 10 mL of sterile water for injection. Reconstituted solutions are drawn into a syringe; in one alternative, the syringe can be fitted with an 0.2-μm syringe filter, although the use of the syringe filter is not required and is optional. When a syringe filter is used, the syringe is only fitted with the syringe filter after the reconstituted solution is drawn into the syringe. One unit of bisantrene dihydrochloride is then filtered directly into a 500-mL i.v. infusion bag (from which 12 mL are removed from the 500-mL initial volume) and the syringe filter is washed into the infusion bag using 2 mL of sterile water for injection. Alternatively, two units of bisantrene dihydrochloride are filtered into a 1-L infusion bag (from which 24 mL are removed from the 1 L initial volume). The contents of the infusion bag are then administered to a patient in need of treatment with bisantrene through an i.v. infusion set containing an 0.2-μm in-line filter. Infusion is continued for 2 hours at a rate such that an adult patient receives a total dosage of 250 mg/m² body surface area.

Accordingly, one aspect of the present invention is a method for preparing bisantrene dihydrochloride units for delivery to a patient in need of treatment with bisantrene dihydrochloride comprising the steps of:

(1) preparing an initial stock solution of bisantrene dihydrochloride;

(2) filtering the initial stock solution of bisantrene dihydrochloride;

(3) aliquoting the initial stock solution of bisantrene dihydrochloride into vials; and

(4) lyophilizing the aliquoted stock solution in the vials.

Typically, the initial stock solution of bisantrene dihydrochloride is prepared in sterile water for injection.

Typically, the initial stock solution is prepared at a temperature of about 20° C. to about 25° C. Alternatively, the initial stock solution can be prepared at a temperature of about 4° C.

Typically, the initial stock solution is prepared at a concentration of between about 25 mg/mL and about 40 mg/mL, such as at any concentration from about 25 mg/mL to about 40 mg/mL, including, but not limited to, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, or any value between these values. Preferably, the initial stock solution is prepared at a concentration of about 40 mg/m L.

The initial stock solution is filtered through 1 to 3 filters. When the initial stock solution is filtered through one filter, the filter has a filtration cutoff of about 0.2 μm. When the initial stock solution is filtered through two filters, the first filter has a filtration cutoff of about 1 to 2 μm, and the second filter has a filtration cutoff of about 0.2 μm. When the initial stock solution is filtered through three filters, the first filter has a filtration cutoff of about 4 to 6 μm, the second filter has a filtration cutoff of about 1 to 2 μm, and the third filter has a filtration cutoff of about 0.2 μm.

As described above, the vials can be glass vials or plastic vials. Typically, when glass vials are used, they are silanized. Typically, the silanization is performed by coating the interior of the vials with an organofunctional alkoxysilane selected from the group consisting of (3-aminopropyl)-triethoxysilane, (3-aminopropyl)-diethoxymethylsilane, (3-aminopropyl)-dimethyl-ethoxysilane, (3-aminopropyl)-trimethoxysilane, (3-glycidoxypropyl)-dimethyl-ethoxysilane, (3-mercaptopropyl)-trimethoxysilane, (3-mercaptopropyl)-methyl dimethoxysilane, and derivatives thereof. When plastic vials are used, typically the plastic is selected from the group consisting of cyclic olefin polymer (COP) plastic, cyclic olefin copolymer (COC) plastic, high-density polyethylene plastic, and high-density non-nucleated polypropylene plastic.

Typically, the volume of stock solution aliquoted into each vial is consistent with delivery of about 295 mg of bisantrene dihydrochloride into each vial. Typically, the volume of stock solution aliquoted into each vial is from about 5.0 mL to about 7.5 mL based on the concentration of the initial stock solution. Preferably, the volume of stock solution aliquoted into each vial is from about 5.625 mL to about 6.875 mL based on the concentration of the initial stock solution. Typically, the vials are of a volume of from about 8 mL to about 12 mL; more typically, the vials are of a volume of from about 9 mL to about 11 mL; preferably, the vials are of 10-mL volume.

The sealed vial can be a vial of a photoprotective color, such as amber. However, although photoprotective vials, such as amber-colored vials, can be used, their use is not essential as the lyophilizer is shielded from light and the finished vials can be packaged in light-protective cardboard boxes or other light-protective packaging.

Another aspect of the invention is a method for delivering bisantrene dihydrochloride units to a patient in need of treatment with bisantrene dihydrochloride comprising the steps of:

(1) reconstituting the contents of a bisantrene dihydrochloride unit vial with sterile water;

(2) filtering the reconstituted bisantrene dihydrochloride into a suitable i.v. infusion vehicle; and

(3) infusing into a patient a therapeutic volume of the bisantrene dihydrochloride-infusion vehicle formulation.

Typically, the bisantrene dihydrochloride units comprise about 295 mg of lyophilized bisantrene dihydrochloride. Typically, the contents of a bisantrene dihydrochloride unit vial are reconstituted with about 9 mL to about 11 mL of sterile water, preferably about 10 mL of sterile water.

In one alternative, the filter is a sterile syringe filter. Typically, the sterile syringe filter has a filtration cutoff in a range of from about 0.15 μm to about 0.25 μm. Preferably, the sterile syringe filter has a filtration cutoff in a range of from about 0.175 μm to about 0.225 μm. More preferably, the sterile syringe filter has a filtration cutoff of about 0.2 μm.

Typically, the suitable i.v. infusion vehicle is 5% dextrose in water. Typically, a volume of the i.v. infusion vehicle equivalent to the volume of reconstituted bisantrene dihydrochloride and any filter wash volume is removed before filtration of the reconstituted bisantrene dihydrochloride into the i.v. infusion vehicle. In one alternative, the filter is washed into the i.v. infusion vehicle with an additional volume of sterile water. Typically, the additional volume of sterile water is about 1 mL to about 3 mL. Preferably, the additional volume of sterile water is about 2 mL.

Typically, the volume of the i.v. infusion vehicle is selected from the group consisting of 500 mL and 1 L. When the volume of the i.v. infusion vehicle is 500 mL, typically, a single vial of lyophilized bisantrene dihydrochloride is reconstituted and filtered into the i.v. infusion vehicle. When the volume of the i.v. infusion vehicle is 1 L, typically, two vials of lyophilized bisantrene dihydrochloride are reconstituted and filtered into the i.v. infusion vehicle.

In another alternative, the bisantrene dihydrochloride-infusion vehicle formulation is infused into a patient through an i.v. infusion set containing an in-line filter. Typically, the in-line filter has a filtration cutoff in a range of from about 0.15 μm to about 0.25 μm. Preferably, the in-line filter has a filtration cutoff in a range of from about 0.175 μm to about 0.225 μm. More preferably, the in-line filter has a filtration cutoff of about 0.2 μm.

Typically, the duration of the infusion is from about 1.5 hours to 2.5 hours. Preferably, the duration of the infusion is from about 1.75 hours to 2.25 hours. More preferably, the duration of the infusion is about 2.0 hours.

Typically, the dosage received by the patient is from about 200 mg/m² to about 300 mg/m² body surface area. Preferably, the dosage received by the patient is from about 225 mg/m² to about 275 mg/m² body surface area. More preferably, the dosage received by the patient is about 250 mg/m² body surface area. The selected dosage level for bisantrene depends upon a variety of pharmacokinetic factors including the duration of administration, the rates of excretion and metabolism of bisantrene, the severity of the condition, such as the status of the malignancy being treated, other health considerations affecting the subject, and the status of liver and kidney function of the subject. It also depends on other drugs, compounds and/or materials used in combination with the bisantrene, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000. Optimal dosages for a given set of conditions can be ascertained by those skilled in the art using conventional dosage-determination tests in view of the experimental data for bisantrene.

Typically, the bisantrene dihydrochloride is administered to the patient to treat a malignancy selected from the group consisting of: breast cancer, acute myelocytic leukemia, acute lymphocytic leukemia of childhood, myelodysplastic syndrome, chronic myelocytic leukemia, chronic lymphocytic leukemia, Hodgkin's lymphoma, non-Hodgkin's lymphoma, mycosis fungoides, prostate cancer, lung small-cell carcinoma, lung non-small cell carcinoma, glioblastoma, a malignancy characterized by overexpressed topoisomerase II, a malignancy characterized by overexpressed and/or mutated EGFR, ovarian cancer, renal cancer, melanoma, gastric cancer, adrenal cancer, head and neck cancer, hepatocellular cancer, hypernephroma, bladder cancer, myeloma, and localized polyp stage colon cancer. The breast cancer can be, but is not limited to, refractory breast cancer, triple-negative breast cancer, or breast cancer characterized by overexpressed Her-2-neu. The acute myelocytic leukemia can be, but is not limited to, acute myelocytic leukemia of childhood. The prostate cancer can be, but is not limited to, androgen-resistant prostate cancer. The small-cell carcinoma of the lung can be characterized by either wild-type or mutated EGFR. The non-small-cell carcinoma of the lung can be characterized by either wild-type or mutated EGFR. The glioblastoma can be, but is not limited to, glioblastoma that is resistant to one or both of the following agents: temozolomide or bevacizumab. Additionally, the glioblastoma can be characterized by EGFR Variant III. However, the bisantrene dihydrochloride can also be administered to treat other diseases and conditions, including malignancies, hyperproliferative conditions other than malignancies, and conditions other than hyperproliferative conditions.

Methods according to the present application can include administration of a therapeutically effective quantity of at least one additional therapeutic agent to treat the malignancy or other condition treatable by administration of bisantrene dihydrochloride. What constitutes a “therapeutically effective quantity” of any additional therapeutic agent can be determined by one of skill in the art by consideration of a variety of pharmacokinetic factors including the duration of administration, the rates of excretion and metabolism of the additional agent, the severity of the condition, such as the status of the malignancy being treated, other health considerations affecting the subject, and the status of liver and kidney function of the subject. It also depends on other drugs, compounds and/or materials used in combination with the bisantrene and the one or more additional agents, as well as the age, weight, condition, general health and prior medical history of the subject being treated, and like factors. The term “therapeutically effective quantity” used in reference to the administration of bisantrene dihydrochloride or another therapeutic agent is not to be interpreted as implying a cure for any disease or condition being treated. As stated previously, in the absence of compatibility studies, administration of bisantrene is recommended as a single drug and bisantrene must not be mixed with other products. Therefore, when one or more additional agents are administered besides bisantrene dihydrochloride, the one or more additional agents are administered separately from the bisantrene dihydrochloride, such as in one or more pharmaceutical compositions. Further details on suitable pharmaceutical compositions for administration of additional agents are provided below.

When the malignancy is breast cancer, the additional therapeutic agent can be selected from the group consisting of tamoxifen, anastrozole, letrozole, cyclophosphamide, docetaxel, paclitaxel, methotrexate, fluorouracil, and trastuzumab, but is not limited to those agents.

When the malignancy is chronic myelocytic leukemia, the additional therapeutic agent can be selected from the group consisting of: cytarabine; hydroxyurea; an alkylating agent selected from the group consisting of melphalan, chlorambucil, cyclophosphamide, mechlorethamine, uramustine, ifosfamide, bendamustine, carmustine, lomustine, streptozotocin, busulfan, procarbazine, altretamine, dacarbazine, temozolomide, and mitozolomide; interferon alfa 2b; a steroid selected from the group consisting of prednisone and prednisolone; and a Bcr-Abl tyrosine kinase inhibitor selected from the group consisting of imatinib, dasatinib, bosutinib, and radotinib, but is not limited to those agents.

When the malignancy is myelodysplastic syndrome, the additional therapeutic agent can be selected from the group consisting of 5-azacytidine, decitabine, and lenalidomide, but is not limited to those agents.

When the malignancy is mycosis fungoides, the additional therapeutic agent can be selected from the group consisting of a corticosteroid, etretinate, arotinoid, acitretin, isotretinoin, bexarotene, carmustine, methotrexate, vorinostat, interferon α, denileukin diftitox, mechlorethamine, depsipeptide, panobinostat, belinostat, alemtuzumab, zanolimumab, cyclophosphamide, chlorambucil, etoposide, dexamethasone, doxorubicin, bleomycin, and vinblastine, but is not limited to those agents.

When the malignancy is ovarian cancer, the additional therapeutic agent can be selected from the group consisting of: a platinum-containing antineoplastic drug selected from the group consisting of cisplatin, carboplatin, oxaliplatin, nedaplatin, triplatin, phenanthriplatin, picoplatin, and satraplatin; paclitaxel; topotecan; gemcitabine; etoposide; and bleomycin, but is not limited to those agents.

When the malignancy is renal cancer, the additional therapeutic agent can be selected from the group consisting of everolimus, torisel, nexavar, sunitinib, axitinib, inferferon, interleukin-2, pazopanib, sorafenib, nivolumab, cabozanitib, and levanitib, but is not limited to those agents.

When the malignancy is lung small-cell carcinoma, the additional therapeutic agent can be selected from the group consisting of cyclophosphamide, cisplatin, etoposide, vincristine, paclitaxel, and carboplatin, but is not limited to those agents.

When the malignancy is lung non-small-cell carcinoma, the additional therapeutic agent can be selected from the group consisting of cisplatin, erlotinib, gefitinib, afatinib, crizotinib, bevacizumab, carboplatin, paclitaxel, nivolumab, and pembrolizumab, but is not limited to those agents.

When the malignancy is Hodgkin's lymphoma, the additional therapeutic agent can be selected from the group consisting of mechlorethamine, vincristine, prednisone, procarbazine, bleomycin, vinblastine, dacarbazine, etoposide, and cyclophosphamide, but is not limited to those agents.

When the malignancy is non-Hodgkin's lymphoma, the additional therapeutic agent can be selected from the group consisting of cyclophosphamide, vincristine, and prednisone, but is not limited to those agents.

When the malignancy is acute myelocytic leukemia, the additional therapeutic agent can be selected from the group consisting of cytarabine, fludarabine, all-trans-retinoic acid, interleukin-2, and arsenic trioxide, but is not limited to those agents.

When the malignancy is melanoma, the additional therapeutic agent can be selected from the group consisting of temozolomide, dacarbazine, interferon, interleukin-2, ipilimumab, pembrolizumab, nivolumab, vemurafenib, dabrafenib, and trametinib, but is not limited to those agents.

When the malignancy is adrenal cancer, the additional therapeutic agent can be selected from the group consisting of mitotane, cisplatin, etoposide, and streptozotocin, but is not limited to those agents.

When the malignancy is head and neck cancer, the additional therapeutic agent can be selected from the group consisting of paclitaxel, carboplatin, cetuximab, docetaxel, cisplatin, and 5-fluorouracil, but is not limited to those agents.

When the malignancy is hepatocellular cancer, the additional therapeutic agent can be selected from the group consisting of tamoxifen, octreoside, synthetic retinoids, cisplatin, 5-fluorouracil, interferon, taxol, and sorafenib, but is not limited to those agents.

When the malignancy is hypernephroma, the additional therapeutic agent can be selected from the group consisting of nivolumab, everolimus, sorafenib, axitinib, lenvatinib, temsirolimus, sunitinib, pazopanib, interleukin-2, cabozanitib, bevacizumab, interferon α, ipilimumab, atezolizumab, varilumab, durvalumab, tremelimumab, and avelumab, but is not limited to those agents.

When the malignancy is bladder cancer, the additional therapeutic agent can be selected from the group consisting of cisplatin, 5-fluorouracil, mitomycin C, gemcitabine, methotrexate, vinblastine, carboplatin, paclitaxel, docetaxel, ifosfamide, and pemetrexed, but is not limited to those agents.

When the malignancy is acute myelocytic leukemia of childhood, the additional therapeutic agent can be selected from the group consisting of methotrexate, nelarabine, asparaginase, blinatumomab, cyclophosphamide, clofarabine, cytarabine, dasatinib, methotrexate, imatinib, pomatinib, vincristine, 6-mercaptopurine, pegaspargase, and prednisone, but is not limited to those agents.

When the malignancy is acute lymphocytic leukemia, the additional therapeutic agent can be selected from the group consisting of asparaginase, vincristine, dexamethasone, methotrexate, 6-mercaptopurine, cytarabine, hydrocortisone, 6-thioguanine, prednisone, etoposide, cyclophosphamide, mitoxantrone, and teniposide, but is not limited to those agents.

When the malignancy is chronic lymphocytic leukemia, the additional therapeutic agent can be selected from the group consisting of fludarabine, cyclophosphamide, rituximab, vincristine, prednisolone, bendamustine, alemtuzumab, ofatumumab, obinutuzumab, ibrutinib, idelalisib, and venetoclax, but is not limited to those agents.

When the malignancy is prostate cancer, the additional therapeutic agent can be selected from the group consisting of temozolomide, docetaxel, cabazitaxel, bevacizumab, thalidomide, prednisone, sipuleucel-T, abiraterone, and enzalutamide, but is not limited to those agents.

When the malignancy is glioblastoma, the additional therapeutic agent can be selected from the group consisting of temozolomide and bevacizumab, but is not limited to those agents.

When the malignancy is myeloma, the additional therapeutic agent can be selected from the group consisting of bortezomib, lenalidomide, dexamethasone, melphalan, prednisone, thalidomide, and cyclophosphamide, but is not limited to those agents.

When the malignancy is a malignancy characterized by overexpressed topoisomerase II, the additional therapeutic agent can be selected from the group consisting of etoposide, teniposide, doxorubicin, daunorubicin, mitoxantrone, amsacrine, ellipticine, aurintricarboxylic acid, and HU-331 (3-hydroxy-2-[(1R)-6-isopropenyl-3-methyl-cyclohex-2-en-1-yl]-5-pentyl-1,4-benzoquinone), but is not limited to those agents.

When the malignancy is a malignancy characterized by overexpressed and/or mutated EGFR, the additional therapeutic agent can be selected from the group consisting of gefitinib, erlotinib, afatinib, brigatinib, icotinib, cetuximab, osimertinib, panitumumab, zalutumumab, nimotuzumab, matuzumab, and lapatinib, but is not limited to those agents.

When the malignancy is gastric cancer, the additional therapeutic agent can be selected from the group consisting of 5-fluorouracil, capecitabine, carmustine, semustine, doxorubicin, mitomycin C, cisplatin, taxotere, and trastuzumab, but is not limited to those agents.

When the malignancy is localized polyp stage colon cancer, the additional therapeutic agent can be selected from the group consisting of tegafur/uracil, capecitabine, 5-fluorouracil, oxaliplatin, irinotecan, bevacizumab, cetuximab, panitumumab, and folinic acid, but is not limited to those agents.

Methods for administration of those additional agents are known in the art, including suitable dosages, dose frequencies, routes of administration, durations of administration, and administration in pharmaceutical compositions, including carriers or excipients. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000. Optimal dosages for a given set of conditions can be ascertained by those skilled in the art using conventional dosage-determination tests in view of the experimental data for an agent. Typically, as stated above, when one or more additional therapeutic agents are administered, they are administered separately from the bisantrene dihydrochloride. The one or more additional therapeutic agents can be administered in one or more pharmaceutical compositions which contain at least one pharmaceutically acceptable carrier, excipient, or filler as is known in the art. When two or more additional therapeutic agents are administered in pharmaceutical compositions, each additional therapeutic agent can be administered in its own pharmaceutical compositions, or, if the additional therapeutic agents are compatible, two or more additional therapeutic agents can be administered in a single pharmaceutical composition.

In some alternatives, the additional therapeutic agent can be a pyrimidine analog antimetabolite which is administered in a therapeutically effective quantity. Suitable pyrimidine analog antimetabolites include, but are not limited to, a pyrimidine analog metabolite selected from the group consisting of cytarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, and 2′-C-cyano-2′-deoxy-13-arabinofuranosylcytosine. Preferably, the pyrimidine analog antimetabolite is selected from the group consisting of cytarabine, 5-azacytidine, gemcitabine, floxuridine, 5-fluorouracil, capecitabine, and 6-azauracil. A particularly preferred pyrimidine analog antimetabolite is cytarabine.

In another alternative, the bisantrene dihydrochloride can be administered with a therapeutically effective quantity of an additional agent selected from the group consisting of: an agent inducing immunoactivity; an agent inducing macrophage activation; a cytokine; an agent inhibiting telomerase; an agent inhibiting survivin; an agent inhibiting methylation or modulating demethylation; an adjuvant; an antibody; an innate or adaptive immune stimulator; a checkpoint inhibitor; a mTOR antagonist; an Akt inhibitor; a notch inhibitor; an Hsp90 inhibitor; a phosphatidylinositide 3-kinase inhibitor; a kinase inhibitor; taxane; and taxol.

Cytokines include, but are not limited to, interleukin-1, interleukin-2, interleukin-4, interleukin-5, interleukin-6, interferon-γ, TGF-β, interleukin-3, interleukin-7, GMCSF, MIP-1a, MIP-1b, MCP-1, RANTES, interleukin-8, lymphotactin, fractalkine, interleukin-10, interleukin-13, interferon-α, and interferon-β.

Telomerase inhibitors include, but are not limited to, 7-deaza-2′-deoxyguanosine, antisense oligonucleotides, imetelstat, BPPA (2,6-bis(3-piperidinopropionamido)anthraquinone), (−)-epigallocatechin gallate, H-7 (2,6-bis(3-piperidinopropionamido)anthraquinone), β-rubromycin, and BIBR1532 (2-[[(2E)-3-(2-naphthalenyl)-1-oxo-2-butenyl1-yl]amino]benzoic acid).

Inhibitors of survivin include, but are not limited to: antisense oligonucleotides; YM155 (septantronium bromide); 5-aminoimidazole-4-carboxamide-1-13-D-furanoside (AICAR); arctigenin; cephalochromin; FL118 (7-ethyl-7-hydroxy-10H-[1,3]dioxolo[4,5-g]pyrano[3′,4′:6,7]indolizino[1,2-b]quinoline-8,11(7H,13H)-dione); flavopiridol; KPT-185 (isopropyl (Z)-3-(3-(3-methoxy-5-(trifluoromethyl)phenyl)-1H-1,2,4-triazol-1-yl)acrylate); lapatinib; MK-2206 (8-(4-(1-aminocyclobutyl)phenyl)-9-phenyl-[1,2,4]triazolo[3,4-f][1,6]naphthyridin-3(2H)-one); panepoxydone; piperine; purvalanol A; shepherdin; terameprocol; UC112 (5-[(phenylmethoxy)methyl]-7-(1-pyrrolidinylmethyl)-8-quinolinol); NSC80467 (2-methyl-1-(2-methylpropyl)-3-[2-(4-nitrophenyl)-2-oxoethyl]benzo[f]benzimidazol-3-ium-4,9-dione bromide); SPC3042 (a locked antisense nucleic acid designed as an antisense 16-mer LNA gapmer) (J. B. Hansen et al., “SPC3042: A Proapoptotic Survivin Inhibitor,” Mol. Cancer Ther. 7: 2736-2745 (2008) targeting the region comprising the stop codon of the open reading frame in exon 4 of the survivin transcript); NU6140 (4-(6-cyclohexylmethoxy-9H-purin-2-ylamino)-N,N-diethylbenzamide); toxoflavin; gambogic acid; LLP-3 (4-(3,5-bis(benzyloxy)phenyl)-6-(5-chloro-2-hydroxyphenyl)-2-oxo-1,2-dihydropyridine-3-carbonitrile); gataparsen; (6S,9S)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide; 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate; tetra-O-methyl-nordihydroguaiaretic acid; butane-bridge-modified tetra-O-methyl-nordihydroguaiaretic acids, including 1,4-bis[3,4-bis[3-(piperidin-1-yl)propoxy]phenyl]-butane; tetra-substituted nordihydroguaiaretic acid derivatives via ether bonds or carbamate bonds; tetraglycinyl nordihydroguaiaretic acid; LY2181308 (an antisense nucleotide); dichloroacetic acid; and ICG-001 ((6S,9aS)-6-(4-hydroxybenzyl)-N-benzyl-8-(naphthalen-1-ylmethyl)-4,7-dioxo-hexahydro-2H-pyrazino[1,2-a]pyrimidine-1(6H)-carboxamide). Other survivin inhibitors and methods for inhibiting the expression of survivin are disclosed in U.S. Pat. No. 8,455,488 to Odagami et al., U.S. Pat. No. 8,318,815 to Huang et al., U.S. Pat. No. 8,232,277 to Chen et al., U.S. Pat. No. 8,178,527 to Chen et al., U.S. Pat. No. 7,959,923 to You et al., United States Patent Application Publication No. 20120088770 by Odagami et al., United States Patent Application Publication No. 20110263607 by Kouji et al., United States Patent Application Publication No. 20110092459 by Odagami et al., United States Patent Application Publication No. 20090304695 by He et al., United States Patent Application Publication No. 20090202539 by You et al., United States Patent Application Publication No. 20080267951 by You et al., United States Patent Application Publication No. 20060040883 by You et al., and United States Patent Application Publication No. 20030125287 by Kandimalla et al. Additional survivin inhibitors are disclosed in: U.S. Pat. No. 7,710,068 to Berezov et al., incorporated herein by this reference, and include compounds of Formula (A-I):

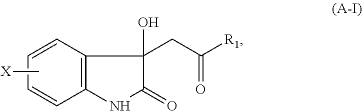

wherein: X is hydrogen, halogen, hydroxyl, alkoxy, or C₁-C₄ linear or branched alkyl; and R₁ is C₁-C₆ linear or branched alkyl or cycloalkyl optionally substituted with halogen, nitro, amine, or dioxole). Inhibitors or modulators or survivin are also disclosed in U.S. Pat. No. 8,026,355 to Hansen et al. (oligonucleotides, particularly antisense oligonucleotides, targeted to nucleic acids encoding survivin) and in U.S. Pat. No. 7,910,742 to Wendt et al. (a compound selected from the group consisting of tert-butyl 4-(((5-chloro-3-(4-(2-chloro-5-(trifluoromethyl)phenyl)-5-cyano-6-oxo-1,6-dihydropyridin-2-yl)-2-hydroxybenzyl)(methyl)amino)carbonyl)-1-piperidinecarboxylate; N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-piperidinecarboxamide; 1-acetyl-N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N,4-dimethyl-4-piperidinecarboxamide; tert-butyl 4-(((5-chloro-3-(4-(2-chloro-5-(trifluoromethyl)phenyl)-5-cyano-6-oxo-1,6-dihydropyridin-2-yl)-2-hydroxybenzyl)(methyl)amino)carbonyl)-4-phenyl-1-piperidinecarboxylate; N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-phenyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(4-pyridinyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-1-(4-cyanophenyl)-N-methyl-4-piperidinecarboxamide; 1-(4-acetylphenyl)-N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-piperidinecarboxamide; 1-acetyl-N-(5-chloro-3-(4-(2-chloro-5-(trifluoromethyl)phenyl)-5-cyano-6-oxo-1,6-dihydropyridin-2-yl)-2-hydroxybenzyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-1-(methoxyacetyl)-N-methyl-4-piperidinecarboxamide; 1-butyryl-N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(2-m ethylbutanoyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(4,4,4-trifluorobutanoyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(4,4,4-trifluorobutanoyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(tetrahydro-2-furanylcarbonyl)-4-piperidinecarboxamide; 1-(3-butynoyl)-N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(3-nitropropanoyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-1-(cyclopropylcarbonyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-1-(cyclopropylacetyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-1-(cyclohexylcarbonyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-propyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(2-phenylethyl)-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(2-(2,6,6-trimethyl-1-cyclohexen-1-yl)ethyl)-4-piperidinecarboxamide; 1-(2-(benzyloxy)ethyl)-N-(5-chloro-3-(4-(2-chloro-5-(trifluoromethyl)phenyl)-5-cyano-6-oxo-1,6-dihydropyridin-2-yl)-2-hydroxybenzyl)-N-methyl-4-piperidinecarboxamide; N-(5-chloro-3-(4-(2-chloro-5-(trifluoromethyl)phenyl)-5-cyano-6-oxo-1,6-dihydropyridin-2-yl)-2-hydroxybenzyl)-N-methyl-1-(3-(5-methyl-2-furyl)butyl)-4-piperidinecarboxamide; 1-acetyl-N-((4′-chloro-5-(4-(2-chloro-5-(trifluoromethyl)phenyl)-5-cyano-6-oxo-1,6-dihydropyridin-2-yl)-4-hydroxy(1,1′-biphenyl)-3-yl)methyl)-N-methyl-4-piperidinecarboxamide; and 1-acetyl-N-(3-(4-(2-chloro-5-(trifluoromethyl)phenyl)-5-cyano-6-oxo-1,6-dihydropyridin-2-yl)-5-cyclopentyl-2-hydroxybenzyl)-N-methyl-4-piperidinecarboxamide.

Agents inhibiting methylation include, but are not limited to, 5′-azacytidine, 5-aza-2′-deoxycytidine, zebularine, L-methionine, apicidine, hydralazine, procainamide, and antisense oligonucleotides directed against mRNA for DNA methyltransferase. Agents that inhibit DNA methylation are described in PCT Patent Application Publication No. WO 2009/106549 by Geroni et al. Additional drugs that modulate DNA demethylation include inhibitors of histone deacetylase (HDAC). These compounds include, but are not limited to, compounds disclosed in PCT Patent Application Publication No. WO 02/22577 by Bair et al., including, but not limited to, N-hydroxy-3-[4-[[(2-hydroxyethyl)[2-(1H-indol-3-yl)ethyl]-amino]methyl]phenyl]-2E-2-propenamide, suberoylanilide hydroxamic acid, 4-(2-amino-phenylcarbamoyl)-benzyl]-carbamic acid pyridine-3-ylmethyl ester and derivatives thereof, butyric acid, pyroxamide, trichostatin A, oxamflatin, apicidin, depsipeptide, depudecin, trapoxin, HC toxin, and sodium phenylbutyrate.

Adjuvants include, but are not limited to, GM-CSF, poly-ICLC (carboxymethylcellulose, polyinosinic-polycytidylic acid, and poly L-lysine), nanoparticles, microparticles, aluminum salts, squalene, QS-21 (a plant extract from Quillaja saponaria containing water-soluble triterpene glycosides), virosomes, IL-2, IL-7, IL-21, and type 1 interferons.

Checkpoint inhibitors include, but are not limited to, ipilimumab, nivolumab, pembrolizumab, atezolizumab, avelumab, durvalumab, and spartalizumab.

mTOR inhibitors include, but are not limited to: sirolimus; temsirolimus; everolimus; rapamune; ridaforolimus; AP23573 (deforolimus); CCI-779 (rapamycin 42-ester with 3-hydroxy-2-(hydroxymethyl)-2-methylpropionic acid); AZD8055 ((5-(2,4-bis((S)-3-methylmorpholino)pyrido[2,3-d]pyrimidin-7-yl)-2-methoxyphenyl)methanol); PKI-587 (1-(4-(4-(dimethylamino)piperidine-1-carbonyl)phenyl)-3-(4-(4,6-dimorpholino-1,3,5-triazin-2-yl)phenyl)urea); NVP-BEZ235 (2-methyl-2-{4-[3-methyl-2-oxo-8-(quinolin-3-yl)-2,3-dihydro-1H-imidazo[4,5-c]quinolin-1-yl]phenyl}propanenitrile); LY294002 ((2-(4-morpholinyl)-8-phenyl-4H-1-benzopyran-4-one); 40-O-(2-hydroxyethyl)-rapamycin; ABT578 (zotarolimus); biolimus-7; biolimus-9; AP23675; AP23841; TAFA-93; 42-O-(methyl-D-glucosylcarbonyl)rapamycin; 42-O-[2-(methyl-D-glucosylcarbonyloxy)ethyl]rapamycin; 31-O-(methyl-D-glucosylcarbonyl)rapamycin; 42-0-(2-hydroxyethyl)-31-O-(methyl-D-glucosylcarbonyl)rapamycin; 42-O-(2-O-methyl-D-fructosylcarbonyl)rapamycin; 42-O-[2-(2-O-methyl-D-fructosylcarbonyloxy)ethyl]rapamycin; 42-O-(2-O-methyl-L-fructosylcarbonyl)rapamycin; 42-O-[2-(2-O-methyl-L-fructosylcarbonyloxy)ethyl]rapamycin; 31-O-(2-O-methyl-D-fructosylcarbonyl)rapamycin; 42-O-(2-hydroxyethyl)-31-O-(2-O-methyl-D-fructosylcarbonyl)rapamycin; 31-O-(2-O-methyl-L-fructosylcarbonyl)rapamycin; 42-O-(2-hydroxyethyl)-31-O-(2-O-methyl-L-fructosylcarbonyl)rapamycin; 42-O-(D-allosylcarbonyl)rapamycin; 42-O-[2-(D-allosylcarbonyloxy)ethyl]rapamycin; 42-O-(L-allosylcarbonyl)rapamycin; 42-O-[2-(L-allosylcarbonyloxy)ethyl]rapamycin; 31-O-(D-allosylcarbonyl)rapamycin; 42-O-(2-hydroxyethyl)-31-O-(D-allosylcarbonyl)rapamycin; 31-O-(L-allosylcarbonyl)rapamycin; 42-O-(2-hydroxyethyl)-31-O-(L-allosylcarbonyl)rapamycin; 42-O-(D-fructosylcarbonyl)rapamycin; 42-O-[2-(D-fructosylcarbonyloxy)ethyl]rapamycin; 42-O-(L-fructosylcarbonyl)rapamycin; 42-O-[2-(L-fructosylcarbonyloxy)ethyl]rapamycin; 31-O-(D-fructosylcarbonyl)rapamycin; 42-O-(2-hydroxyethyl)-31-O-(D-fructosylcarbonyl)rapamycin; 31-O-(L-fructosylcarbonyl)rapamycin; 42-O-(2-hydroxyethyl)-31-O-(L-fructosylcarbonyl)rapamycin; 42-O-(D-fucitolylcarbonyl)rapamycin; 42-O-[2-(D-fucitolylcarbonyloxy)ethyl]rapamycin; 42-O-(L-fucitolylcarbonyl)rapamycin; 42-O-[2-(L-fucitolylcarbonyloxy)ethyl]rapamycin; 31-O-(D-fucitolylcarbonyl)rapamycin; 42-O-(2-hydroxyethyl)-31-O-(D-fucitolylcarbonyl)rapamycin; 31-O-(L-fucitolylcarbonyl)rapamycin; 42-O-(2-hydroxyethyl)-31-O-(L-fucitolylcarbonyl)rapamycin; 42-O-(D-glucalylcarbonyl)rapamycin; 42-O-[2-(D-glucalylcarbonyloxy)ethyl]rapamycin; 42-O-(D-glucosylcarbonyl)rapamycin; 42-O-[2-(D-glucosylcarbonyloxy)ethyl]rapamycin; glucosylcarbonyl)rapamycin; 42-O-[2-(L-glucosylcarbonyloxy)ethyl]rapamycin; 31-O-(D-glucalylcarbonyl)rapamycin; 42-O-(2-hydroxyethyl)-31-O-(D-glucalylcarbonyl)rapamycin; 31-O-(D-glucosylcarbonyl)rapamycin; 42-O-(2-hydroxyethyl)-31-O-(D-glucosylcarbonyl)rapamycin; 31-O-(L-glucosylcarbonyl)rapamycin; 42-O-(2-hydroxyethyl)-31-O-(L-glucosylcarbonyl)rapamycin; sorbosylcarbonyl)rapamycin; 42-O-(D-sorbosylcarbonyl)rapamycin; sorbosylcarbonyl)rapamycin; 31-O-(D-sorbosylcarbonyl)rapamycin; 42-O-[2-(L-sorbosylcarbonyloxy)ethyl]rapamycin; 42-O-[2-(D-sorbosylcarbonyloxy)ethyl]rapamycin; 42-O-(2-hydroxyethyl)-31-O-(D-sorbosylcarbonyl)rapamycin; 42-O-(2-hydroxyethyl)-31-O-(L-sorbosylcarbonyl)rapamycin; 42-O-(D-lactalylcarbonyl)rapamycin; 42-O-[2-(D-lactalylcarbonyloxy)ethyl]rapamycin; 31-O-(D-lactalylcarbonyl)rapamycin; 42-O-(2-hydroxyethyl)-31-O-(D-lactalylcarbonyl)rapamycin; 42-O-(D-sucrosylcarbonyl)rapamycin; 42-O-[2-(D-sucrosylcarbonyloxy)ethyl]rapamycin; 31-O-(D-sucrosylcarbonyl)rapamycin; 42-O-(2-hydroxyethyl)-31-O-(D-sucrosylcarbonyl)rapamycin; 42-O-(D-gentobiosylcarbonyl)rapamycin; 42-O-[2-(D-gentobiosylcarbonyloxy)ethyl]rapamycin; 31-O-(D-gentobiosylcarbonyl)rapamycin; 42-O-(2-hydroxyethyl)-31-O-(D-gentobiosylcarbonyl)rapamycin; 42-O-(D-cellobiosylcarbonyl)rapamycin; 42-O-[2-(D-cellobiosylcarbonyloxy)ethyl]rapamycin; 31-O-(D-cellobiosylcarbonyl)rapamycin; 42-O-(2-hydroxyethyl)-31-O-(D-cellobiosylcarbonyl)rapamycin; 42-O-(D-turanosylcarbonyl)rapamycin; 42-O-[2-(D-turanosylcarbonyloxy)ethyl]rapamycin; 31-O-(D-turanosylcarbonyl)rapamycin; 42-O-(2-hydroxyethyl)-31-O-(D-turanosylcarbonyl)rapamycin; 42-O-(D-palatinosylcarbonyl)rapamycin; 42-O-[2-(D-palatinosylcarbonyloxy)ethyl]rapamycin; 31-O-(D-palatinosylcarbonyl)rapamycin; 42-O-(2-hydroxyethyl)-31-O-(D-palatinosylcarbonyl)rapamycin; 42-O-(D-isomaltosylcarbonyl)rapamycin; 42-O-[2-(D-isomaltosylcarbonyloxy)ethyl]rapamycin; 31-O-(D-isomaltosylcarbonyl)rapamycin; 42-O-(2-hydroxyethyl)-31-O-(D-isomaltosylcarbonyl)rapamycin; 42-O-(D-maltulosylcarbonyl)rapamycin; 42-O-[2-(D-maltulosylcarbonyloxy)ethyl]rapamycin; 42-O-(D-maltosylcarbonyl)rapamycin; 42-O-[2-(D-maltosylcarbonyloxy)ethyl]rapamycin; 31-O-(D-maltulosylcarbonyl)rapamycin; 42-O-(2-hydroxyethyl)-31-O-(D-maltulosylcarbonyl)rapamycin; 31-O-(D-maltosylcarbonyl)rapamycin; 42-O-(2-hydroxyethyl)-31-O-(D-maltosylcarbonyl)rapamycin; 42-O-(D-lactosylcarbonyl)rapamycin; 42-O-[2-(D-lactosylcarbonyloxy)ethyl]rapamycin; 31-O-(methyl-D-lactosylcarbonyl)rapamycin; 42-O-(2-hydroxyethyl)-31-O-(methyl-D-lactosylcarbonyl)rapamycin; 42-O-(D-melibiosylcarbonyl)rapamycin; 31-O-(D-melibiosylcarbonyl)rapamycin; 42-O-(2-hydroxyethyl)-31-O-(D-melibiosylcarbonyl)rapamycin; 42-O-(D-leucrosylcarbonyl)rapamycin; 42-O-[2-(D-leucrosylcarbonyloxy)ethyl]rapamycin; 31-O-(D-leucrosylcarbonyl)rapamycin; 42-O-(2-hydroxyethyl)-31-O-(D-leucrosylcarbonyl)rapamycin; 42-O-(D-raffinosylcarbonyl)rapamycin; 42-O-[2-(D-raffinosylcarbonyloxy)ethyl]rapamycin; 31-O-(D-raffinosylcarbonyl)rapamycin; 42-O-(2-hydroxyethyl)-31-O-(D-raffinosylcarbonyl)rapamycin; 42-O-(D-isomaltotriosylcarbonyl)rapamycin; 42-O-[2-(D-isomaltosylcarbonyloxy)ethyl]rapamycin; 31-O-(D-isomaltotriosylcarbonyl)rapamycin; 42-O-(2-hydroxyethyl)-31-O-(D-isomaltotriosylcarbonyl)rapamycin; 42-O-(D-cellotetraosylcarbonyl)rapamycin; 42-O-[2-(D-cellotetraosylcarbonyloxy)ethyl]rapamycin; 31-O-(D-cellotetraosylcarbonyl)rapamycin; 42-O-(2-hydroxyethyl)-31-O-(D-cellotetraosylcarbonyl)rapamycin; 42-O-(valiolylcarbonyl)rapamycin; 42-O-[2-(D-valiolylcarbonyloxy)ethyl]rapamycin; 31-O-(valiolylcarbonyl)rapamycin; 42-O-(2-hydroxyethyl)-31-O-(valiolylcarbonyl)rapamycin; 42-O-(valiolonylcarbonyl)rapamycin; 42-O-[2-(D-valiolonylcarbonyloxy)ethyl]rapamycin; 31-O-(valiolonylcarbonyl)rapamycin; 42-O-(2-hydroxyethyl)-31-O-(valiolonylcarbonyl)rapamycin; 42-O-(valienolylcarbonyl)rapamycin; 42-O-[2-(D-valienolylcarbonyloxy)ethyl]rapamycin; 31-O-(valienolylcarbonyl)rapamycin; 42-O-(2-hydroxyethyl)-31-O-(valienolylcarbonyl)rapamycin; 42-O-(valienoneylcarbonyl)rapamycin; 42-O-[2-(D-valienoneylcarbonyloxy)ethyl]rapamycin; 31-O-(valienoneylcarbonyl)rapamycin; 42-O-(2-hydroxyethyl)-31-O-(valienoneylcarbonyl)rapamycin; PI-103 (3-[4-(4-morpholinyl)pyrido[3′,2′:4,5]furo[3,2-d]pyrimidin-2-yl]-phenol); KU-0063794 ((5-(24(2R,6S)-2,6-dimethylmorpholino)-4-morpholinopyrido[2,3-d]pyrimidin-7-yl)-2-methoxyphenyl)methanol); PF-04691502 (2-amino-8-((1r,4r)-4-(2-hydroxyethoxy)cyclohexyl)-6-(6-methoxypyridin-3-O-4-methylpyrido[2,3-d]pyrimidin-7(8H)-one); CH132799; RG7422 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one); Palomid 529 (3-(4-methoxybenzyloxy)-8-(1-hydroxyethyl)-2-methoxy-6H-benzo[c]chromen-6-one); PP242 (2-(4-amino-1-isopropyl-1H-pyrazolo[3,4-d]pyrimidin-3-yl)-1H-indol-5-ol); XL765 (N-[4-[[[3-[(3,5-dimethoxyphenyl)amino]-2-quinoxalinyl]amino]sulfonyl]phenyl]-3-methoxy-4-methyl-benzamide); GSK1059615 ((Z)-5-((4-(pyridin-4-yl)quinolin-6-yl)methylene)thiazolidine-2,4-dione); PKI-587 (1-(4-(4-(dimethylamino)piperidine-1-carbonyl)phenyl)-3-(4-(4,6-dimorpholino-1,3,5-triazin-2-yl)phenyl)urea); WAY-600 (6-(1H-indol-5-yl)-4-morpholino-1-(1-(pyridin-3-ylmethyl)piperidin-4-yl)-1H-pyrazolo[3,4-d]pyrimidine); WYE-687 (methyl 4-(4-morpholino-1-(1-(pyridin-3-ylmethyl)piperidin-4-yl)-1H-pyrazolo[3,4-d]pyrimidin-6-yl)phenylcarbamate); WYE-125132 (N-[4-[1-(1,4-dioxaspiro[4.5]dec-8-yl)-4-(8-oxa-3-azabicyclo[3.2.1]oct-3-yl)-1H-pyrazolo[3,4-d]pyrimidin-6-yl]phenyl]-N′-methyl-urea); and WYE-354 (4-[6-[4-[(methoxycarbonyl)amino]phenyl]-4-(4-morpholinyl)-1H-pyrazolo[3,4-d]pyrimidin-1-yl]-1-piperidinecarboxylic acid methyl ester). Additional inhibitors of mTOR are described in the following United States patents and patent applications: U.S. Pat. No. 8,461,157 to Cai et al.; U.S. Pat. No. 8,440,662 to Smith et al.; U.S. Pat. No. 8,436,012 to Ohtsuka et al.; U.S. Pat. No. 8,394,818 to Gray et al.; U.S. Pat. No. 8,362,241 to D'Angelo et al.; U.S. Pat. No. 8,314,111 to Chen et al.; U.S. Pat. No. 8,309,546 to Nakayama et al. (including 6-morpholinopurine derivatives); U.S. Pat. No. 8,268,819 to Jin et al.; U.S. Pat. No. 8,211,669 to Reed et al.; U.S. Pat. No. 8,163,755 to Jin et al.; U.S. Pat. No. 8,129,371 to Zask et al.; U.S. Pat. No. 8,097,622 to Nakayama et al.; U.S. Pat. No. 8,093,050 to Cho et al.; U.S. Pat. No. 8,008,318 to Beckmann et al.; U.S. Pat. No. 7,943,767 to Chen et al.; U.S. Pat. No. 7,923,555 to Chen et al.; U.S. Pat. No. 7,897,608 to Wilkinson et al.; U.S. Pat. No. 7,700,594 to Chen et al.; U.S. Pat. No. 7,659,274 to Crew et al.; U.S. Pat. No. 7,655,673 to Zhang et al. (39-desmethoxyrapamycin); U.S. Pat. No. 7,648,996 to Beckman et al.; U.S. Pat. No. 7,504,397 to Hummersone et al.; U.S. Pat. No. 7,169,817 to Pan et al.; U.S. Pat. No. 7,160,867 to Abel et al. (carbohydrate derivatives of rapamycin); U.S. Pat. No. 7,091,213 to Metcalf III et al. (“rapalogs”); United States Patent Application Publication No. 2013/0079303 by Andrews et al.; and United States Patent Application Publication No. 2013/0040973 by Vannuchi et al.

Akt inhibitors include, but are not limited to: triciribine: RX-0201 (a 20-mer oligonucleotide); perifosine; PX-316 ((R)-2-methoxy-3-(octadecyloxy)propyl ((1R,2R,3S,4R,6R)-2,3,4,6-tetrahydroxycyclohexyl) hydrogen phosphate); API-1 (4-amino-5,8-dihydro-5-oxo-8-β-D-ribofuranosyl-pyrido[2,3-d]pyrimidine-6-carboxamide); SR13668 (diethyl 6-methoxy-5,7-dihydroindolo[2,3-b]carbazole-2,10-dicarboxylate); AZD5363 (4-amino-N-[(1S)-1-(4-chlorophenyl)-3-hydroxypropyl]-1-(7H-pyrrolo[2,3-d]pyrimidin-4-yl)-4-piperidinecarboxamide); miltefosine; GSK690693 (4-(2-(4-amino-1,2,5-oxadiazol-3-yl)-1-ethyl-74(S)-piperidin-3-ylmethoxy)-1H-imidazo[4,5-c]pyridin-4-yl)-2-methylbut-3-yn-2-ol); A-443654 ((2S)-1-(1H-indol-3-yl)-3-[5-(3-methyl-2H-indazol-5-Opyridin-3-yl]oxypropan-2-amine); and SR13668 (diethyl 6-methoxy-5,7-dihydroindolo[2,3-b]carbazole-2,10-dicarboxylate). Additional inhibitors of Akt are described in the following United States patents and patent applications: U.S. Pat. No. 8,450,305 to Winssinger et al.; U.S. Pat. No. 8,445,509 to Miyamoto et al. (N-[4-({2-[(cyclopropylcarbonyl)amino]imidazo[1,2-a]pyridin-6-yl}oxy)-3-fluorophenyl]-N′-phenylcyclopropane-1,1-dicarboxamide, N-[4-({2-[(cyclopropylcarbonyl)amino]imidazo[1,2-a]pyridin-6-yl}oxy)-3-fluorophenyl]-6-methyl-2-oxo-1-phenyl-1,2-dihydropyridine-3-carboxamide, N-[4-({2-[(cyclopropylcarbonyl)amino]imidazo[1,2-a]pyridin-6-yl}oxy)-3-fluorophenyl]-1-(4-fluorophenyl)-6-methyl-2-oxo-1,2-dihydropyridine-3-carboxamide, and N-[5-({2-[(cyclopropylcarbonyl)amino]imidazo[1,2-a]pyridin-6-yl}oxy)pyridin-2-yl]-6-(4-fluorophenyl)-5-methylpyridine-2-carboxamide 1-oxide); U.S. Pat. No. 8,436,002 to Beight et al. ((R)-5-methyl-4-(4-(1-(2-(pyrrolidin-1-yl)ethyl)-4-(3,3,3-trifluoropropyl)-1H-imidazol-2-yl)piperidin-1-yl)-5,6-dihydropyrido[2,3-d]pyrimidin-7(8H)-one; (R)-4-(4-(4-ethyl-1-(2-(pyrrolidin-1-yl)ethyl)-1H-imidazol-2-yl)piperidin-1-yl)-5-methyl-5,6-dihydropyrido[2,3-d]pyrimidin-7(8H)-one; and (R)-4-(4-(1-(2-(azetidin-1-yl)ethyl)-4-(2,2,2-trifluoroethyl)-1H-imidazol-2-yl)piperidin-1-yl)-5-(trifluoromethyl)-5,6-dihydropyrido[2,3-d]pyrimidin-7(8H)-one); U.S. Pat. No. 8,420,690 to Seefeld et al. (N-(2-amino-1-phenylethyl)-5-(1-methyl-1H-pyrazol-5-yl)-3-thiophenecarboxamide; N-[2-amino-1-(phenylmethyl)ethyl]-5-(1-methyl-1H-pyrazol-5-yl)-3-thiophenecarboxamide; N-((1S)-2-amino-1-{[2-(trifluoromethyl)phenyl]methyl}ethyl)-5-(1-methyl-1H-pyrazol-5-yl)-3-thiophenecarboxamide; N-{(1S)-2-amino-1-[(2-fluorophenyl)methyl]ethyl}-5-(1-methyl-1H-pyrazol-5-yl)-3-thiophenecarboxamide; N-{(1S)-2-amino-1-[(2-chlorophenyl)methyl]ethyl}-5-(1-methyl-1H-pyrazol-5-yl)-3-thiophenecarboxamide; N[1-(aminomethyl)-2-methyl-2-phenylpropyl]-5-(1-methyl-1H-pyrazol-5-yl)-3-thiophenecarboxamide; N[2-amino-1-(1-naphthalenyl)ethyl]-5-(1-methyl-1H-pyrazol-5-yl)-3-thiophenecarboxamide; N[2-amino-1-(phenylmethyl)ethyl]-2-(3-furanyl)-5-(1-methyl-1H-pyrazol-5-yl)-3-thiophenecarboxamide; N-((1S)-2-amino-1-{[2-(trifluoromethyl)phenyl]methyl}ethyl)-5-(1-methyl-1H-pyrazol-5-yl)-3-furancarboxamide; N-((1S)-2-amino-1-{[2-(trifluoromethyl)phenyl]methyl}ethyl)-1-methyl-5-(1-methyl-1H-pyrazol-5-yl)-1H-pyrrole-3-carboxamide; N-((1S)-2-amino-1-{[2-(trifluoromethyl)phenyl]methyl}ethyl)-2-chloro-1-methyl-5-(1-methyl-1H-pyrazol-5-yl)-1H-pyrrole-3-carboxamide; and N-((1S)-2-amino-1-{[2-(trifluoromethyl)phenyl]methyl}ethyl)-2-chloro-5-(4-chloro-1-methyl-1H-pyrazol-5-yl)-1-methyl-1H-pyrrole-3-carboxamide); U.S. Pat. No. 8,420,678 to Mahadevan et al.; U.S. Pat. No. 8,410,158 to Seefeld et al. (N-{(1S)-2-amino-1-[(3,4-difluorophenyl)methyl]ethyl]-5-chloro-4-(4-chloro-1-methyl-1H-pyrazol-5-yl)-2-furancarboxamide); U.S. Pat. No. 8,338,434 to Seefeld et al. (N-[2-amino-1-(phenylmethyl)ethyl]-4-(1-methyl-1H-pyrazol-5-yl)benzamide; N-[2-amino-1-(phenylmethyl)ethyl]-2-chloro-4-(1-methyl-1H-pyrazol-5-yl)benzamide; N-[2-amino-1-(phenylmethyl)ethyl]-3-chloro-4-(1-methyl-1H-pyrazol-5-yl)benzamide; N-[2-amino-1-(phenylmethyl)ethyl]-4-(1-methyl-1H-pyrazol-5-yl)-3-(trifluoromethyl)benzamide; N-((1S)-2-amino-1-{[2-(trifluoromethyl)phenyl]methyl}ethyl)-3-chloro-4-(1-methyl-1H-pyrazol-5-yl)benzamide; N-[2-amino-1-(phenylmethyl)ethyl]-3-methyl-4-(1-methyl-1H-pyrazol-5-yl)benzamide; N-((1S)-2-amino-1-{[2-(trifluoromethyl)phenyl]methyl}ethyl)-3-fluoro-4-(1-methyl-1H-pyrazol-5-yl)benzamide; N-((1S)-2-amino-1-{[2-(trifluoromethyl)phenyl]methyl}ethyl)-3-methyl-4-(1-methyl-1H-pyrazol-5-yl)benzamide; N-[2-amino-1-(phenylmethyl)ethyl]-3-hydroxy-4-(1-methyl-1H-pyrazol-5-yl)benzamide; N-[2-amino-1-(phenylmethyl)ethyl]-3-fluoro-4-(1-methyl-1H-pyrazol-5-yl)benzamide; N-((1S)-2-amino-1-{[2-(trifluoromethyl)phenyl]methyl}ethyl)-4-chloro-5-(1-methyl-1H-pyrazol-5-yl)-2-pyridinecarboxamide; N-[2-amino-1-(phenylmethyl)ethyl]-4-(4-chloro-1-methyl-1H-pyrazol-5-yl)-3-fluorobenzamide; N-[2-amino-1-(phenylmethyl)ethyl]-3-chloro-4-(4-chloro-1-methyl-1H-pyrazol-5-yl)benzamide; N-((1S)-2-amino-1-{[2-(trifluoromethyl)phenyl]methyl}ethyl)-3-fluoro-4-(4-chloro-1-methyl-1H-pyrazol-5-yl)benzamide; N-((1S)-2-amino-1-{[2-(trifluoromethyl)phenyl]methyl}ethyl)-3-chloro-4-(4-chloro-1-methyl-1H-pyrazol-5-yl)benzamide; N-[2-amino-1-(phenylmethyl)ethyl]-3-bromo-4-(1-methyl-1H-pyrazol-5-yl)benzamide; N-((1S)-2-amino-1-{[2-(trifluoromethyl)phenyl]methyl}ethyl)-3-bromo-4-(1-methyl-1H-pyrazol-5-yl)benzamide; 3-amino-N-[3-chloro-4-(1-methyl-1H-pyrazol-5-yl)phenyl]-2-phenylpropanamide; and 3-amino-N-[3-chloro-4-(1-methyl-1H-pyrazol-5-yl)phenyl]-2-(phenylmethyl)propanamide); U.S. Pat. No. 8,273,782 to Seefeld et al. (N-{(1S)-2-amino-1-[(3-fluorophenyl)methyl]ethyl}-5-chloro-4-(4-chloro-1-methyl-1H-pyrazol-5-yl)-2-thiophenecarboxamide); U.S. Pat. No. 8,263,357 to Reed; U.S. Pat. No. 8,242,147 to Dumas et al.; U.S. Pat. No. 8,183,249 to Cheng et al.; U.S. Pat. No. 8,124,630 to Riedl et al.; U.S. Pat. No. 8,114,870 to Xiao et al.; U.S. Pat. No. 8,101,623 to Luke et al. ((S)-4-amino-N-(1-(4-chlorophenyl)-3-hydroxypropyl)-1-(7H-pyrrolo[2,3-d]pyrimidin-4-yl)piperidine-4-carboxamide); U.S. Pat. No. 8,067,412 to Winssinger et al.; U.S. Pat. No. 7,998,977 to Joseph et al. (4-[5-(2-amino-ethanesulfonyl)-isoquinolin-7-yl]-phenol); U.S. Pat. No. 7,982,037 to Bebbington et al.; U.S. Pat. No. 7,951,820 to Bebbington et al.; U.S. Pat. No. 7,987,623 to Riedl et al. (N-(4-chloro-3-(trifluoromethyl)phenyl)-N′-(4-(2-(N-methylcarbamoyl)-4-pyridyloxy)phenyl)urea); U.S. Pat. No. 7,879,853 to Stadlwieser et al. (N-[4-(6-dibenzofuran-4-yl-pyrimidin-4-ylamino)-phenyl]-4-morpholin-4-yl-benzamide, N-[4-(6-dibenzofuran-4-yl-pyrimidin-4-ylamino)-phenyl]-4-dimethylamino-benzamide, N-[4-(6-dibenzofuran-4-yl-pyrimidin-4-ylamino)-phenyl]-4-(4-methyl-piperazin-1-ylmethyl)-benzamide, N-[4-(6-dibenzofuran-4-yl-pyrimidin-4-ylamino)-phenyl]-2-(4-dimethyl-amino-phenyl)-acetamide, N-[4-(6-dibenzofuran-4-yl-pyrimidin-4-ylamino)-phenyl]-2-dimethylamino-benzamide, N-[4-(6-dibenzofuran-4-yl-pyrimidin-4-ylamino)-phenyl]-3-pyrrolidin-1-ylbenzamide, N-[4-(6-dibenzofuran-4-yl-pyrimidin-4-ylamino)-phenyl]-benzamide, 4-tert-butyl-N-[4-(6-dibenzofuran-4-yl-pyrimidin-4-ylamino)-phenyl]-benzamide, 3,4-dichloro-N-[4-(6-dibenzofuran-4-yl-pyrimidin-4-ylamino)-phenyl]-benzamide, N-[4-(6-dibenzofuran-4-yl-pyrimidin-4-ylamino)-phenyl]-3-dimethylamino-benzamide, N-[4-(6-dibenzofuran-4-yl-pyrimidin-4-ylamino)-phenyl]-isonicotinamide, N-[4-(6-dibenzofuran-4-yl-pyrimidin-4-ylamino)-phenyl]-4-dimethylaminomethyl-benzamide, N-[4-(6-dibenzofuran-4-yl-pyrimidin-4-ylamino)-phenyl]-4-morpholin-4-ylmethyl-benzamide, N-[4-(6-dibenzofuran-4-yl-pyrimidin-4-ylamino)-phenyl]-4-(4-methylpiperazin-1-yl)-benzamide, N-[4-(6-dibenzofuran-4-yl-pyrimidin-4-ylamino)-phenyl]-6-morpholin-4-yl-nicotinamide, N-[4-(6-dibenzofuran-4-yl-pyrimidin-4-ylamino)-phenyl]-3-[3-methoxy-1-(2-methoxyethyl)-propyl]-benzamide, tert-butyl N-{4-[4-(6-dibenzofuran-4-yl-pyrimidin-4-ylamino)-phenylcarbamoyl]-benzyl}-carbamate, tert-butyl N-{2-[4-(6-dibenzofuran-4-yl-pyrimidin-4-ylamino)-phenylcarbamoyl]-phenyl}-carbamate, tert-butyl N-{3-[4-(6-dibenzofuran-4-yl-pyrimidin-4-ylamino)-phenylcarbamoyl]-phenyl}-carbamate, tert-butyl 3-{4-[4-(6-dibenzofuran-4-yl-pyrimidin-4-ylamino)-phenylcarbamoyl]-phenyl}-piperidin-1-carboxylate, tert-butyl N-(4-{[4-(6-dibenzofuran-4-yl-pyrimidin-4-ylamino)-phenylcarbamoyl]-methyl}-phenyl)-carbamate, tert-butyl N-{3-[4-(6-dibenzofuran-4-yl-pyrimidin-4-ylamino)-phenylcarbamoyl]-benzyl}-carbamate, tert-butyl N-(2-{4-[4-(6-dibenzofuran-4-yl-pyrimidin-4-ylamino)-phenylcarbamoyl]phenyl}-ethyl)-carbamate, tert-butyl N-{2-[4-(6-dibenzofuran-4-yl-pyrimidin-4-ylamino)-phenylcarbamoyl]-pyridin-4-ylmethyl}-carbamate, tert-butyl N-{4-[4-(6-dibenzofuran-4-yl-pyrimidin-4-ylamino)-phenylcarbamoyl]-benzyl}-methyl-carbamate, tert-butyl {5-[4-(6-dibenzofuran-4-yl-pyrimidin-4-ylamino)-phenylcarbamoyl]-pyridin-2-ylmethyl}-carbamate, tert-butyl {4-[4-(6-dibenzofuran-4-yl-pyrimidin-4-ylamino)-phenylcarbamoyl]-pyridin-2-ylmethyl}-carbamate, tert-butyl (4-{[4-(6-dibenzofuran-4-yl-pyrimidin-4-ylamino)-phenylcarbamoyl]-methyl}-benzyl)-carbamate, tert-butyl N-(1-{4-[4-(6-dibenzofuran-4-yl-pyrimidin-4-ylamino)-phenylcarbamoyl]-phenyl}-1-methyl-ethyl)-carbamate, tert-butyl N-(2-{3-[4-(6-dibenzofuran-4-yl-pyrimidin-4-ylamino)-phenylcarbamoyl]-phenyl}-ethyl)-carbamate, {4-[4-(6-dibenzofuran-4-yl-pyrimidin-4-ylamino)-phenylcarbamoyl]-phenyl}-(2-methoxyethyl)-carbamate, tert-butyl N-{4-[4-(6-dibenzofuran-4-yl-pyrimidin-4-ylamino)-phenylcarbamoyl]-3-fluorobenzyl}carbamate, {6-[4-(6-dibenzofuran-4-yl-pyrimidin-4-ylamino)-phenylcarbamoyl]-pyridin-2-ylmethyl}-carbamate, tert-butyl N-{5-[4-(6-dibenzofuran-4-yl-pyrimidin-4-ylamino)-phenylcarbamoyl]-pyridin-3-ylmethyl}-carbamate, 3-cyano-N-[4-(6-dibenzofuran-4-yl-pyrimidin-4-ylamino)-phenyl]-benzamide, 3-carbamimidoyl-N-[4-(6-dibenzofuran-4-yl-pyrimidin-4-ylamino)-phenyl]-benzamide, 4-cyano-N-[4-(6-dibenzofuran-4-yl-pyrimidin-4-ylamino)-phenyl]-benzamide, 4-carbamimidoyl-N-[4-(6-dibenzofuran-4-yl-pyrimidin-4-ylamino)-phenyl]-benzamide, 4-aminomethyl-N-[4-(6-dibenzofuran-4-yl-pyrimidin-4-ylamino)-phenyl]-benzamide, 2-amino-N-[4-(6-dibenzofuran-4-yl-pyrimidin-4-ylamino)-phenyl]-benzamide, 3-amino-N-[4-(6-dibenzofuran-4-yl-pyrimidin-4-ylamino)-phenyl]-benzamide, N-[4-(6-dibenzofuran-4-yl-pyrimidin-4-ylamino)-phenyl]-4-piperidin-3-yl-benzamide, 2-(4-amino-phenyl)-N-[4-(6-dibenzofuran-4-yl-pyrimidin-4-ylamino)-phenyl]-acetamide, 3-aminomethyl-N-[4-(6-dibenzofuran-4-yl-pyrimidin-4-ylamino)-phenyl]-benzamide, 4-(2-amino-ethyl)-N-[4-(6-dibenzofuran-4-yl-pyrimidin-4-ylamino)-phenyl]-benzamide, N-[4-(6-dibenzofuran-4-yl-pyrimidin-4-ylamino)-phenyl]-4-methylaminomethyl-benzamide, 6-aminomethyl-N-[4-(6-dibenzofuran-4-yl-pyrimidin-4-ylamino)-phenyl]-nicotinamide, 2-aminomethyl-N-[4-(6-dibenzofuran-4-yl-pyrimidin-4-ylamino)-phenyl]-isonicotinamide, 2-(4-aminomethyl-phenyl)-N-[4-(6-dibenzofuran-4-yl-pyrimidin-4-ylamino)-phenyl]-acetamide, 4-(1-amino-1-methyl-ethyl)-N-[4-(6-dibenzofuran-4-yl-pyrimidin-4-ylamino)-phenyl]-benzamide, 3-(2-amino-ethyl)-N-[4-(6-dibenzofuran-4-yl-pyrimidin-4-ylamino)-phenyl]-benzamide, N-[4-(6-dibenzofuran-4-yl-pyrimidin-4-ylamino)-phenyl]-4-(2-methoxyethylamino)benzamide, 4-aminomethyl-N-[4-(6-dibenzofuran-4-yl-pyrimidin-4-ylamino)-phenyl]-2-fluorobenzamide, 5-aminomethyl-N-[4-(6-dibenzofuran-4-yl-pyrimidin-4-ylamino)-phenyl]-nicotinamide, 3-amino-N-[4-(6-dibenzofuran-4-yl-pyrimidin-4-ylamino)-phenyl]-2-(3,4-dichloro-phenyl)-propionamide, 5-aminomethyl-pyridine-2-carboxylic acid [4-(6-dibenzofuran-4-yl-pyrimidin-4-ylamino)-phenyl]-amide, 1,2,3,4-tetrahydro-isoquinoline-6-carboxylic acid [4-(6-dibenzofuran-4-yl-pyrimidin-4-ylamino)-phenyl]-amide, tert-butyl {4-[3-(6-dibenzofuran-4-yl-pyrimidin-4-ylamino)-phenylcarbamoyl]-benzyl}-carbamate, tert-butyl N-(2-{4-[3-(6-dibenzofuran-4-yl-pyrimidin-4-ylamino)-phenylcarbamoyl]-phenyl}-ethyl)-carbamate, tert-butyl N-{2-[3-(6-dibenzofuran-4-yl-pyrimidin-4-ylamino)-phenylcarbamoyl]-phenyl}-carbamate, tert-butyl {3-[3-(6-dibenzofuran-4-yl-pyrimidin-4-ylamino)-phenylcarbamoyl]-phenyl}-carbamate, tert-butyl N-{3-[3-(6-dibenzofuran-4-yl-pyrimidin-4-ylamino)-phenylcarbamoyl]-benzyl}-carbamate, tert-butyl N-{4-[3-(6-dibenzofuran-4-yl-pyrimidin-4-ylamino)-phenylcarbamoyl]-phenyl}-carbamate, tert-butyl N-{4-[3-(6-dibenzofuran-4-yl-pyrimidin-4-ylamino)-4-methyl-phenylcarbamoyl]-benzyl}-carbamate, N-[3-(6-dibenzofuran-4-yl-pyrimidin-4-ylamino)-phenyl]-4-dimethylamino-benzamide, N-[3-(6-dibenzofuran-4-yl-pyrimidin-4-ylamino)-phenyl]-4-dimethylaminomethyl-benzamide, N-[3-(6-dibenzofuran-4-yl-pyrimidin-4-ylamino)-phenyl]-3-dimethylamino-benzamide, 4-aminomethyl-N-[3-(6-dibenzofuran-4-yl-pyrimidin-4-ylamino)-phenyl]-benzamide, 4-(2-amino-ethyl)-N-[3-(6-dibenzofuran-4-yl-pyrimidin-4-ylamino)-phenyl]-benzamide, 2-amino-N-[3-(6-dibenzofuran-4-yl-pyrimidin-4-ylamino)-phenyl]-benzamide, 3-amino-N-[3-(6-dibenzofuran-4-yl-pyrimidin-4-ylamino)-phenyl]-benzamide, 3-aminomethyl-N-[3-(6-dibenzofuran-4-yl-pyrimidin-4-ylamino)-phenyl]-benzamide, 4-amino-N-[3-(6-dibenzofuran-4-yl-pyrimidin-4-ylamino)-phenyl]-benzamide, 4-aminomethyl-N-[3-(6-dibenzofuran-4-yl-pyrimidin-4-ylamino)-4-methyl-phenyl]-benzamide, 3-amino-N-[3-(6-dibenzofuran-4-yl-pyrimidin-4-ylamino)-phenyl]-2-(3,4-dichlorophenyl)-propionamide); U.S. Pat. No. 7,807,705 to Chen et al.; U.S. Pat. No. 7,795,290 to Dickson, Jr. et al.; U.S. Pat. No. 7,745,446 to Maier et al.; U.S. Pat. No. 7,691,853 to Bebbington et al.; U.S. Pat. No. 7,652,135 to Binch et al.; U.S. Pat. No. 7,652,027 to Lee et al.; U.S. Pat. No. 7,625,913 to Bebbington et al.; U.S. Pat. No. 7,625,890 to Heerding et al. (4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol; 4-(2-(4-amino-1,2,5-oxadiazol-3-yl)-1-ethyl-7-{[(2S)-2-thiomorpholinylmethyl]oxy}-1H-imidazo[4,5-c]pyridin-4-yl)-2-methyl-3-butyn-2-ol; 4-(2-(4-amino-1,2,5-oxadiazol-3-yl)-1-ethyl-7-{[(2S)-2-morpholinylmethyl]oxy}-1H-imidazo[4,5-c]pyridin-4-yl)-2-methyl-3-butyn-2-ol; and 4-[2-(4-amino-1,2,5-oxadiazol-3-yl)-1-ethyl-7-({[(2R)-6-methyl-2-morpholinyl]methyl}oxy)-1H-imidazo[4,5-c]pyridin-4-yl]-2-methyl-3-butyn-2-ol); U.S. Pat. No. 7,531,556 to Green; U.S. Pat. No. 7,449,477 to Barda et al.; U.S. Pat. No. 7,414,063 to Al-Awar et al.; U.S. Pat. No. 7,410,988 to Dickson, Jr. et al. (2-amidothiazole-based compounds); U.S. Pat. No. 7,390,815 to Davies et al. (pyrazole compounds); U.S. Pat. No. 7,354,919 to Hale et al. (isoxazole compounds); U.S. Pat. No. 7,345,054 to Hale et al.; U.S. Pat. No. 7,304,061 to Hale et al.; U.S. Pat. No. 7,253,187 to Cao et al.; U.S. Pat. No. 7,115,739 to Bebbington et al.; U.S. Pat. No. 7,098,330 to Bebbington et al. (pyrazolylamine-substituted quinazoline compounds); U.S. Pat. No. 7,087,603 to Bebbington et al. (pyrazole compounds); U.S. Pat. No. 7,041,687 to Binch et al. (indazole compounds); U.S. Pat. No. 7,008,948 to Bebbington et al. (fused pyrimidyl pyrazole compounds); U.S. Pat. No. 6,989,385 to Bebbington et al. (pyrazole compounds); U.S. Pat. No. 6,743,791 to Cao et al.; U.S. Pat. No. 6,696,452 to Bebbington et al. (pyrazole compounds); U.S. Pat. No. 6,664,247 to Bebbington et al. ((5-cyclopropyl-2H-pyrazol-3-yl)-[2-(naphtalen-2-ylsulfanyl)-6-phenylpyrimidin-4-yl]-amine; (5-cyclopropyl-2H-pyrazol-3-yl)-[2-(3-methoxycarbonyl-phenylylsulfanyl)-6-phenylpyrimidin-4-yl]-amine; (5-cyclopropyl-2H-pyrazol-3-yl)-[2-(naphthalen-2-ylsulfanyl)-pyrimidin-4-yl]-amine; (5-cyclopropyl-2H-pyrazol-3-yl)-[5,6-dimethyl-2-(naphthalen-2-ylsulfanyl)-pyrimidin-4-yl]-amine; (5-cyclopropyl-2H-pyrazol-3-yl)-[5-methyl-2-(naphthalen-2-ylsulfanyl)-pyrimidin-4-yl]-amine; (5-cyclopropyl-2H-pyrazol-3-yl)-[6-methyl-2-(naphthalen-2-ylsulfanyl)-pyrimidin-4-yl]-amine; (5-cyclopropyl-2H-pyrazol-3-yl)-[6-(morpholin-4-yl)-2-(naphthalen-2-ylsulfanyl)-pyrimidin-4-yl]-amine; (5-cyclopropyl-2H-pyrazol-3-yl)-[6-(1-methylpiperazin-4-yl)-2-(naphthalen-2-ylsulfanyl)-pyrimidin-4-yl]-amine; [6-(2,6-dimethylphenyl)-2-(naphthalen-2-ylsulfanyl)-pyrimidin-4-yl]-(5-methyl-2H-pyrazol-3-yl)-amine; [6-(2-methylphenyl)-2-(naphthalen-2-ylsulfanyl)-pyrimidin-4-yl]-(5-methyl-2H-pyrazol-3-yl)-amine; [2-(4-acetamido-phenylsulfanyl)-6-phenyl-pyrimidin-4-yl]-(5-methyl-2H-pyrazol-3-yl)-amine; (5-methyl-2H-pyrazol-3-yl)-[2-(naphthalen-2-ylsulfanyl)-6-phenyl-pyrimidin-4-yl]-amine; [2-(4-isobutyrylylamino-phenylsulfanyl)-6-phenylpyrimidin-4-yl]-(5-methyl-2H-pyrazol-3-yl)-amine; [6-(4-methylpiperazin-1-yl)-2-methylsulfanyl-pyrimidin-4-yl]-(5-methyl-2H-pyrazol-3-yl)-amine; (5-methyl-2H-pyrazol-3-yl)-[6-phenyl-2-(4-propionylamino-phenylsulfanyl)-pyrimidin-4-yl]-amine; [2-(4-cyclopropanecarbonylamino-phenylsulfanyl)-6-phenylpyrimidin-4-yl]-(5-methyl-2H-pyrazol-3-yl)-amine; (5-methyl-2H-pyrazol-3-yl)-{6-phenyl-2-[4-(propane-1-sulfonylamino)-phenylsulfanyl]-pyrimidin-4-yl}-amine; [2-(4-ethanesulfonylamino-phenylsulfanyl)-6-phenyl-pyrimidin-4-yl]-(5-methyl-2H-pyrazol-3-yl)-amine; [2-(4-acetamidophenyl-sulfanyl)-6-(2-methylphenyl)-pyrimidin-4-yl]-(5-methyl-2H-pyrazol-3-yl)-amine; [2-(4-isobutanecarbonylamino-phenyl-sulfanyl)-6-phenyl-pyrimidin-4-yl]-(5-methyl-2H-pyrazol-3-yl)-amine; [2-(4-acetamido-phenyl-sulfanyl)-5-methyl-6-phenyl-pyrimidin-4-yl]-(5-methyl-2H-pyrazol-3-yl)-amine; [2-(4-acetamido-phenyl-sulfanyl)-6-(4-methoxyphenyl)-pyrimidin-4-yl]-(5-methyl-2H-pyrazol-3-yl)-amine; [6-(3-acetamidophenyl)-2-(4-acetamido-phenyl-sulfanyl)-pyrimidin-4-yl]-(5-methyl-2H-pyrazol-3-yl)-amine; [2-(4-isopropanesulfonylamino-phenyl-sulfanyl)-6-phenyl-pyrimidin-4-yl]-(5-methyl-2H-pyrazol-3-yl)-amine; {2-[4-(2-dimethylamino-acetylamino)-phenylsulfanyl]-6-phenyl-pyrimidin-4-yl}-(5-methyl-2H-pyrazol-3-O-amine; [2-(3-chloro-benzylsulfanyl)-6-morpholin-4-yl-pyrimidin-4-yl]-(5-methyl-2H-pyrazol-3-yl)-amine; [2-(3-chloro-benzylsulfanyl)-6-(2-methoxy-ethylamino)-pyrimidin-4-yl]-(5-methyl-2H-pyrazol-3-yl)-amine; [2-benzylsulfanyl-6-(4-methylpiperazin-1-yl)-pyrimidin-4-yl]-(5-methyl-2H-pyrazol-3-yl)-amine; [2-benzylsulfanyl-6-morpholin-4-yl-pyrimidin-4-yl]-(5-methyl-2H-pyrazol-3-yl)-amine; [2-(3-chloro-benzylsulfanyl)-6-(4-methylpiperazin-1-yl)-pyrimidin-4-yl]-(5-methyl-2H-pyrazol-3-yl)-amine; [2-(4-methoxy-benzylsulfanyl)-6-(4-methylpiperazin-1-yl)-pyrimidin-4-yl]-(5-methyl-2H-pyrazol-3-yl)-amine; [2-(4-acetamido-phenyl-sulfanyl)-6-tert-butyl-pyrimidin-4-yl]-(5-methyl-2H-pyrazol-3-yl)-amine; (5-cyclopropyl-2H-pyrazol-3-yl)-[6-phenyl-2-(4-propionylamino-phenyl-sulfanyl)-pyrimidin-4-yl]-amine; [2-(3-chloro-benzylsulfanyl)-6-(piperidin-1-yl)-pyrimidin-4-yl]-(5-methyl-2H-pyrazol-3-yl)-amine; (5-methyl-2H-pyrazol-3-yl)-{2-[4-(morpholinesulfonyl)-benzylsulfanyl]-6-morpholin-4-yl-pyrimidin-4-yl}-amine; {6-(2-methoxy-ethylamino)-2-[4-(morpholinesulfonyl)-benzylsulfanyl]-pyrimidin-4-yl}-(5-methyl-2H-pyrazol-3-yl)-amine; {6-(4-methylpiperazin-1-yl)-2-[4-(morpholinesulfonyl)-benzylsulfanyl]-pyrimidin-4-yl}-(5-methyl-2H-pyrazol-3-yl)-amine; [6-methoxymethyl-2-(4-propionylamino-phenyl-sulfanyl)-pyrimidin-4-yl]-(5-methyl-2H-pyrazol-3-yl)-amine; [2-(4-methoxycarbonyl-phenyl-sulfanyl)-6-methoxymethyl-pyrimidin-4-yl]-(5-methyl-2H-pyrazol-3-yl)-amine; [2-(3,5-dimethoxy-benzylsulfanyl)-6-morpholin-4-yl-pyrimidin-4-yl]-(5-methyl-2H-pyrazol-3-yl)-amine; [2-(3,5-dimethoxy-benzylsulfanyl)-6-pyrrolidin-4-yl-pyrimidin-4-yl]-(5-methyl-2H-pyrazol-3-yl)-amine; (5-methyl-2H-pyrazol-3-yl)-[6-morpholin-4-yl-2-(naphthalene-2-yl-methylsulfanyl)-pyrimidin-4-yl]-amine; {2-(4-acetamido-phenyl-sulfanyl)-6-[4-(3-dimethylamino-propoxy)phenyl]-pyrimidin-4-yl}-(5-methyl-2H-pyrazol-3-yl)-amine; [2-(4-acetamidophenylsulfanyl)-6-(morpholin-4-yl)-pyrimidin-4-yl]-(5-methyl-2H-pyrazol-3-yl)-amine; [6-hydroxymethyl-2-(4-propionylamino-phenyl-sulfanyl)-pyrimidin-4-yl]-(5-methyl-2H-pyrazol-3-yl)-amine; [2-(4-acetamido-phenyl-sulfanyl)-pyrimidin-4-yl]-(5-methyl-2H-pyrazol-3-yl)-amine; [6-(1-butoxycarbonyl)-2-(4-propionylamino-phenyl-sulfanyl)pyrimidin-4-yl]-(5-methyl-2H-pyrazol-3-yl)-amine; and [6-methoxycarbonyl-2-(4-propionylamino-phenyl-sulfanyl)-pyrimidin-4-yl]-(5-methyl-2H-pyrazol-3-yl)-amine); U.S. Pat. No. 6,660,731 to Bebbington et al.; U.S. Pat. No. 6,653,301 to Bebbington et al. (pyrazole compounds); U.S. Pat. No. 6,649,640 to Hale et al. (isoxazole compounds); U.S. Pat. No. 6,638,926 to Davies et al. (pyrazole compounds); U.S. Pat. No. 6,613,716 to Knegtel et al. (pyrazole compounds); U.S. Pat. No. to U.S. Pat. No. 6,610,677 Davies et al. (pyrazole compounds); U.S. Pat. No. 6,495,582 to Hale et al. (isoxazole compounds, including 4-(4-{3-chloro-4-[(2-dimethylamino-acetylamino)-methyl]-phenyl}-isoxazol-5-yl)-1H-pyrrole-2-carboxylic acid [1-(3,5-dichloro-phenyl)-2-hydroxy-ethyl]-amide); United States Patent Application Publication No. 2013/0034598 by Cheng et al.; United States Patent Application Publication No. 2012/0329793 by Ashwell et al. (substituted imidazopyridinyl compounds); United States Patent Application Publication No. 2012/0329791 by Ashwell et al. (substituted imidazopyridinyl-aminopyridine compounds); United States Patent Application Publication No. 2012/0190707 by Ronai et al.; United States Patent Application Publication No. 2012/0149684 by Beight et al. ((R)-5-methyl-4-(4-(1-(2-(pyrrolidin-1-yl)ethyl)-4-(3,3,3-trifluoropropyl)-1H-imidazol-2-yl)piperidin-1-yl)-5,6-dihydropyrido[2,3-d]pyrimidin-7(8H)-one; (R)-4-(4-(4-ethyl-1-(2-(pyrrolidin-1-yl)ethyl)-1H-imidazol-2-yl)piperidin-1-yl)-5-methyl-5,6-dihydropyrido[2,3-d]pyrimidin-7(8H)-one; and (R)-4-(4-(1-(2-(azetidin-1-yl)ethyl)-4-(2,2,2-trifluoroethyl)-1H-imidazol-2-yl)piperidin-1-yl)-5-(trifluoromethyl)-5,6-dihydropyrido[2,3-d]pyrimidin-7(8H)-one); United States Patent Application Publication No. 2012/0108574 by Ashwell et al.; United States Patent Application Publication No. 2012/0071657 by Bebbington et al.; United States Patent Application Publication No. 2011/0318393 by Ladouceur et al.; United States Patent Application Publication No. 2011/0228142 by Chen et al. (N-{(1S)-2-amino-1-[(3-fluorophenyl)methyl]ethyl}-5-chloro-4-(4-chloro-1-methyl-1H-pyrazol-5-yl)-2-thiophenecarboxamide); United States Patent Application Publication No. 2011/0196009 by Rouse et al. (3-amino-N-[5-chloro-4-(4-chloro-1-methyl-1H-pyrazol-5-yl)-2-thienyl]-2-phenylpropanamide; 3-amino-N-[5-chloro-4-(4-chloro-1-methyl-1H-pyrazol-5-yl)-2-thienyl]-2-(phenylmethyl)propanamide; (2S)-3-amino-N-[5-chloro-4-(4-chloro-1-methyl-1H-pyrazol-5-yl)-2-thienyl]-2-(phenylmethyl)propanamide; (2R)-3-amino-N-[5-chloro-4-(4-chloro-1-methyl-1H-pyrazol-5-yl)-2-thienyl]-2-(phenylmethyl)propanamide; 3-amino-N-[3,5-dichloro-4-(1-methyl-1H-pyrazol-5-yl)-2-thienyl]-2-phenylpropanamide; 3-amino-N-[3,5-dichloro-4-(1-methyl-1H-pyrazol-5-yl)-2-thienyl]-2-(phenylmethyl)propanamide; (2S)-3-amino-N-[5-chloro-4-(4-chloro-1-methyl-1H-pyrazol-5-yl)-2-thienyl]-2-[(3,4-difluorophenyl)methyl]propanamide; (2R)-3-amino-N-[5-chloro-4-(4-chloro-1-methyl-1H-pyrazol-5-yl)-2-thienyl]-2-[(3,4-difluorophenyl)methyl]propanamide; (2S)-3-amino-N-[5-chloro-4-(4-chloro-1-methyl-1H-pyrazol-5-yl)-2-thienyl]-2-[(2-fluorophenyl)methyl]propanamide; (2R)-3-amino-N-[5-chloro-4-(4-chloro-1-methyl-1H-pyrazol-5-yl)-2-thienyl]-2-[(2-fluorophenyl)methyl]propanamide; (2S)-3-amino-N-[5-chloro-4-(4-chloro-1-methyl-1H-pyrazol-5-yl)-2-thienyl]-2-[(4-fluorophenyl)methyl]propanamide; (2R)-3-amino-N-[5-chloro-4-(4-chloro-1-methyl-1H-pyrazol-5-yl)-2-thienyl]-2-[(4-fluorophenyl)methyl]propanamide; (2S)-3-amino-N-[5-chloro-4-(4-chloro-1-methyl-1H-pyrazol-5-yl)-2-thienyl]-2-[(3-fluorophenyl)methyl]propanamide; and (2R)-3-amino-N-[5-chloro-4-(4-chloro-1-methyl-1H-pyrazol-5-yl)-2-thienyl]-2-[(3-fluorophenyl)methyl]propanamide); United States Patent Application Publication No. 2011/0160256 by Rouse et al. (heteropyrrole compounds); United States Patent Application Publication No. 2011/0160255 by Rouse et al. (heteropyrrole compounds); United States Patent Application Publication No. 2011/0129455 by Lin et al. (pyrrole compounds); United States Patent Application Publication No. 2011/0098221 by Lin et al. (heteropyrrole compounds, including N-{(1S)-2-amino-1-[(3-fluorophenyl)methyl]ethyl}-5-(1-methyl-1H-pyrazol-5-yl)-1,3-thiazole-2-carboxamide and N-{(1S)-2-amino-1-[(3,4-difluorophenyl)methyl]ethyl}-4-chloro-5-(1-methyl-1H-pyrazol-5-yl)-1H-imidazole-2-carboxamide); United States Patent Application Publication No. 2011/092423 by Rouse et al. (heteropyrrole compounds, including N-{(1S)-2-amino-1-[(3-fluorophenyl)methyl]ethyl}-2-(1-methyl-1H-pyrazol-5-yl)-1,3-thiazole-5-carboxamide; N-{(1S)-2-amino-1-[(3-fluorophenyl)methyl]ethyl}-2-(4-chloro-1-methyl-1H-pyrazol-5-yl)-1,3-thiazole-5-carboxamide; N-{(1S)-2-amino-1-[(3-fluorophenyl)methyl]ethyl}-2-(1-methyl-1H-pyrazol-5-yl)-1,3-oxazole-5-carboxamide; and N-{(1S)-2-amino-1-[(3-fluorophenyl)methyl]ethyl}-2-(4-chloro-1-methyl-1H-pyrazol-5-yl)-1,3-oxazole-5-carboxamide); United States Patent Application Publication No. 2011/0071182 by Seefeld et al. (heterocyclic carboxamide compounds, including N-{(1S)-2-amino-1-[(3,4-difluorophenyl)methyl]ethyl}-5-chloro-4-(4-chloro-1-methyl-1H-pyrazol-5-yl)-2-furancarboxamide); United States Patent Application Publication No. 2011/0053972 by Seefeld et al. (heterocyclic carboxamide compounds, including N-(2-amino-1-phenylethyl)-5-(1-methyl-1H-pyrazol-5-yl)-3-thiophenecarboxamide; N-[2-amino-1-(phenylmethyl)ethyl]-5-(1-methyl-1H-pyrazol-5-yl)-3-thiophenecarboxamide; N-((1S)-2-amino-1-{[2-(trifluoromethyl)phenyl]methyl}ethyl)-5-(1-methyl-1H-pyrazol-5-yl)-3-thiophenecarboxamide; N-{(1S)-2-amino-1-[(2-fluorophenyl)methyl]ethyl}-5-(1-methyl-1H-pyrazol-5-yl)-3-thiophenecarboxamide; N-{(1S)-2-amino-1-[(2-chlorophenyl)methyl]ethyl}-5-(1-methyl-1H-pyrazol-5-yl)-3-thiophenecarboxamide; N-[1-(aminomethyl)-2-methyl-2-phenylpropyl]-5-(1-methyl-1H-pyrazol-5-yl)-3-thiophenecarboxamide; N-[2-amino-1-(1-naphthalenyl)ethyl]-5-(1-methyl-1H-pyrazol-5-yl)-3-thiophenecarboxamide; N-[2-amino-1-(phenylmethyl)ethyl]-2-(3-furanyl)-5-(1-methyl-1H-pyrazol-5-yl)-3-thiophenecarboxamide; N-((1S)-2-amino-1-{[2-(trifluoromethyl)phenyl]methyl}ethyl)-5-(1-methyl-1H-pyrazol-5-yl)-3-furancarboxamide; N-((1S)-2-amino-1-{[2-(trifluoromethyl)phenyl]methyl}ethyl)-1-methyl-5-(1-methyl-1H-pyrazol-5-yl)-1H-pyrrole-3-carboxamide; N-((1S)-2-amino-1-{[2-(trifluoromethyl)phenyl]methyl}ethyl)-2-chloro-1-methyl-5-(1-methyl-1H-pyrazol-5-yl)-1H-pyrrole-3-carboxamide; and N-((1S)-2-amino-1-{[2-(trifluoromethyl)phenyl]methyl}ethyl)-2-chloro-5-(4-chloro-1-methyl-1H-pyrazol-5-yl)-1-methyl-1H-pyrrole-3-carboxamide); United States Patent Application Publication No. 2010/0267759 by Seefeld et al. (heterocyclic carboxamide compounds); United States Patent Application Publication No. 2010/0137338 by Seefeld et al. (pyrazole compounds, including N-[2-amino-1-(phenylmethyl)ethyl]-5-(1-methyl-1H-pyrazol-5-yl)-2-pyridine-carboxamide; N-[2-amino-1-(phenylmethyl)ethyl]-6-(1-methyl-1H-pyrazol-5-yl)-3-pyridine-carboxamide; N-(2-amino-1-benzylethyl)-5-(1-methyl-1H-pyrazol-5-yl)pyrimidine-2-carboxamide; N-[2-amino-1-(phenylmethyl)ethyl]-4-(1-methyl-1H-pyrazol-5-yl)benzamide; N-[2-amino-1-(phenylmethyl)ethyl]-2-chloro-4-(1-methyl-1H-pyrazol-5-yl)benzamide; N-[2-amino-1-(phenylmethyl)ethyl]-3-chloro-4-(1-methyl-1H-pyrazol-5-yl)benzamide; N-[2-amino-1-(phenylmethyl)ethyl]-4-(1-methyl-1H-pyrazol-5-yl)-3-(trifluoromethyl)benzamide; N-((1S)-2-amino-1-{[2-(trifluoromethyl)phenyl]methyl}ethyl)-5-(1-methyl-1H-pyrazol-5-yl)-2-pyridinecarboxamide; N-((1S)-2-amino-1-{[2-(trifluoromethyl)phenyl]methyl}ethyl)-3-chloro-4-(1-methyl-1H-pyrazol-5-yl)benzamide; N-[2-amino-1-(phenylmethyl)ethyl]-4-chloro-5-(1-methyl-1H-pyrazol-5-yl)-2-pyridinecarboxamide; N-[2-amino-1-(phenylmethyl)ethyl]-6-chloro-5-(1-methyl-1H-pyrazol-5-yl)-2-pyridinecarboxamide; N-[2-amino-1-(phenylmethyl)ethyl]-3-methyl-4-(1-methyl-1H-pyrazol-5-yl)benzamide; N-((1S)-2-amino-1-{[2-(trifluoromethyl)phenyl]methyl}ethyl)-3-fluoro-4-(1-methyl-1H-pyrazol-5-yl)benzamide; N-((1S)-2-amino-1-{[2-(trifluoromethyl)phenyl]methyl}ethyl)-3-methyl-4-(1-methyl-1H-pyrazol-5-yl)benzamide; N-[2-amino-1-(phenylmethyl)ethyl]-3-hydroxy-4-(1-methyl-1H-pyrazol-5-yl)benzamide; N-[2-amino-1-(phenylmethyl)ethyl]-3-fluoro-4-(1-methyl-1H-pyrazol-5-yl)benzamide; N-((1S)-2-amino-1-{[2-(trifluoromethyl)phenyl]methyl}ethyl)-4-chloro-5-(1-methyl-1H-pyrazol-5-yl)-2-pyridinecarboxamide; N-[2-amino-1-(phenylmethyl)ethyl]-4-(4-chloro-1-methyl-1H-pyrazol-5-yl)-3-fluorobenzamide; N-[2-amino-1-(phenylmethyl)ethyl]-3-chloro-4-(4-chloro-1-methyl-1H-pyrazol-5-yl)benzamide; N-((1S)-2-amino-1-{[2-(trifluoromethyl)phenyl]methyl}ethyl)-3-fluoro-4-(4-chloro-1-methyl-1H-pyrazol-5-yl)benzamide; N-((1S)-2-amino-1-{[2-(trifluoromethyl)phenyl]methyl}ethyl)-3-chloro-4-(4-chloro-1-methyl-1H-pyrazol-5-yl)benzamide; N-[2-amino-1-(phenylmethyl)ethyl]-3-bromo-4-(1-methyl-1H-pyrazol-5-yl)benzamide; N-((1S)-2-amino-1-{[2-(trifluoromethyl)phenyl]methyl}ethyl)-3-bromo-4-(1-methyl-1H-pyrazol-5-yl)benzamide; 3-amino-N-[3-chloro-4-(1-methyl-1H-pyrazol-5-yl)phenyl]-2-phenylpropanamide; 3-amino-N-[3-chloro-4-(1-methyl-1H-pyrazol-5-yl)phenyl]-2-(phenylmethyl)propanamide; N-((1S)-2-amino-1-{[2-(trifluoromethyl)phenyl]methyl}ethyl)-4-(1-methyl-1H-1,2,4-triazol-5-yl)benzamide; N-[(1S)-2-amino-1-(phenylmethyl)ethyl]-4-(1-methyl-1H-1,2,4-triazol-5-yl)benzamide; N-{(1S)-2-amino-1-[(3-fluorophenyl)methyl]ethyl}-4-(1-methyl-1H-1,2,4-triazol-5-yl)benzamide; N-((1S)-2-amino-1-{[2-(trifluoromethyl)phenyl]methyl}ethyl)-3-chloro-4-(1-methyl-1H-1,2,4-triazol-5-yl)benzamide; N-{(1S)-2-amino-1-[(3-fluorophenyl)methyl]ethyl}-3-chloro-4-(1-methyl-1H-1,2,4-triazol-5-yl)benzamide; N-[(1S)-2-amino-1-(phenylmethyl)ethyl]-3-chloro-4-(1-methyl-1H-1,2,4-triazol-5-yl)benzamide; N-((1S)-2-amino-1-{[2-(trifluoromethyl)phenyl]methyl}ethyl)-4-(1-methyl-1H-1,2,3-triazol-5-yl)benzamide; N-((1S)-2-amino-1-{[2-(trifluoromethyl)phenyl]methyl}ethyl)-4-(4-chloro-1-methyl-1H-1,2,3-triazol-5-yl)benzamide; N-[(1S)-2-amino-1-(phenylmethyl)ethyl]-4-(4-chloro-1-methyl-1H-1,2,3-triazol-5-yl)benzamide; N-{(1S)-2-amino-1-[(3-fluorophenyl)methyl]ethyl}-4-(4-chloro-1-methyl-1H-1,2,3-triazol-5-yl)benzamide; N-[(1S)-2-amino-1-(phenylmethyl)ethyl]-4-(1-methyl-1H-1,2,3-triazol-5-yl)benzamide; and N-{(1S)-2-amino-1-[(3-fluorophenyl)methyl]ethyl}-4-(1-methyl-1H-1,2,3-triazol-5-yl)benzamide); United States Patent Application Publication No. 2010/0056523 by Heerding et al. (1H-imidazo[4,5-c]pyridin-2-yl compounds including 4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol; 44244-amino-1,2,5-oxadiazol-3-yl)-1-ethyl-7-{[(2S)-2-thiomorpholinylmethyl]oxy}-1H-imidazo[4,5-c]pyridin-4-yl)-2-methyl-3-butyn-2-ol; 4-(2-(4-amino-1,2,5-oxadiazol-3-yl)-1-ethyl-7-{[(2S)-2-morpholinylmethyl]oxy}-1H-imidazo[4,5-c]pyridin-4-yl)-2-methyl-3-butyn-2-ol; and 4-[2-(4-amino-1,2,5-oxadiazol-3-yl)-1-ethyl-7-({[(2R)-6-methyl-2-morpholinyl]methyl}oxy)-1H-imidazo[4,5-c]pyridin-4-yl]-2-methyl-3-butyn-2-ol); PCT Patent Application Publication No. WO 2008/70016 by Kelly et al. (substituted naphthyridine compounds, including (8-[4-(1-aminocyclobutyl)phenyl]-9-phenyl[1,2,4]triazolo[3,4-f]-1,6-naphthyridin-3(2H)-one); and PCT Patent Application Publication No. WO 2007/58850 by Heerding et al. (1H-imidazo[4,5-c]pyridin-2-yl compounds).

Notch inhibitors include, but are not limited to, semagacestat, 7-(S)-[N′(3,5-difluorophenylacetyl)-L-alaninyl]amino-5-methyl-5,7-dihydro-6H-dibenz[b,d]azepin-6-one (YO-01027), and (2R,3S)—N-[(3S)-1-methyl-2-oxo-5-phenyl-2,3-dihydro-1H-1,4-benzodiazepin-3-yl]-2,3-bis(3,3,3-trifluoropropyl)succinamide (BMS-906024). Additional inhibitors of Notch are described in the following United States patents and patent applications: U.S. Pat. No. 8,377,886 to Susztak et al.; U.S. Pat. No. 8,362,075 to Lewis et al.; U.S. Pat. No. 8,343,923 to Long et al. (DAPT (N—[N-(3,5-difluorophenacetyl-L-alanyl)]-S-phenylglycine tert-butyl ester), 1-(S)-endo-N-(1,3,3)-trimethylbicyclo[2.2.1]hept-2-yl)-4-fluorophenyl sulfonamide, WPE-III31C, S-3-[N′-(3,5-difluorophenyl-alpha-hydroxyacetyl)-L-alaninyl]amino-2,3-dihydro-1-methyl-5-phenyl-1H-1,4-benzodiazepin-2-one, (N)—[(S)-2-hydroxy-3-methyl-butyryl]-1-(L-alaninyl)-(S)-1-amino-3-methyl-4,5,6,7-tetrahydro-2H-3-benzazepin-2-one); U.S. Pat. No. 8,242,103 to Lewis et al.; U.S. Pat. No. 8,133,857 to Aikawa; U.S. Pat. No. 8,119,366 to Stylianou; U.S. Pat. No. 7,901,876 to Di Fiore et al.; U.S. Pat. No. 7,837,993 to Conboy et al.; U.S. Pat. No. 7,807,630 to Dang et al.; United States Patent Application Publication No. 2013/0064832 by Aikawa et al.; United States Patent Application Publication No. 2013/0039930 by Alitalo et al.; United States Patent Application Publication No. 2013/0029972 by Hipskind et al. (4,4,4-trifluoro-N-[(1S)-2-[[(75)-5-(2-hydroxyethyl)-6-oxo-7H-pyrido[2,3-d][3]benzazepin-7-yl]amino]-1-methyl-2-oxo-ethyl]butanamide); United States Patent Application Publication No. 2012/0328608 by Siebel (antagonist antibodies and anti-Notch3 NRR (negative regulatory region) antibodies); United States Patent Application Publication No. 2011/0223183 by Kitajewski et al. (fusion proteins as decoy inhibitors); United States Patent Application Publication No. 2011/0178046 by Ross et al. (gamma secretase inhibitors, including semagacestat ((2S)-2-Hydroxy-3-methyl-N-[(1S)-1-methyl-2-oxo-2-[[(1S)-2,3,4,5-tetrahydro-3-methyl-2-oxo-1H-3-benzazepin-1-yl]amino]ethyl]butanamide, also known as LY450139; Eli Lilly and Co.), Compound E ([(2S)-2-{[(3,5-difluorophenyl)acetyl]amino}-N-[(3S)-1-methyl-2-oxo-5-phe-nyl-2,3-dihydro-1H-1,4-benzodiazepin-3-yl]propanamide], available from Alexis Biochemicals), LY411575 (Eli Lilly and Co.), L-685,458 (Sigma-Aldrich), BMS-289948 (4-chloro-N-(2,5-difluorophenyl)-N-((1R)-{4-fluoro-2-[3-(1H-imidazol-1-yl)propyl]phenyl}ethyl)benzenesulfonamide hydrochloride) and BMS-299897 (4-[2-((1R)-1-{[(4-chlorophenyl)sulfonyl]-2,5-difluoroanilino}ethyl)-5-fluorophenyl]butanoic acid) (Bristol Myers Squibb), MK0752 (3-((1r,4s)-4-(4-chlorophenylsulfonyl)-4-(2,5-difluorophenyl)cyclohexyl)propanoic acid), and MRK-003 ((3′R,6R,9R)-5′-(2,2,2-trifluoroethyl)-2-((E)-3-(4-(trifluoromethyl)piperidin-1-yl)prop-1-en-1-yl)-5,6,7,8,9,10-hexahydrospiro[6,9-methanobenzo[8]annulene-11,3′-[1,2,5]thiadiazolidine] 1′,1′-dioxide); United States Patent Application Publication No. 2011/0059096 by Dang et al. (antibodies that bind to epitopes selected from the group consisting of CFNTLGGHS (SEQ ID NO: 14), CVCVNGWTGES (SEQ ID NO: 15), CATAV (SEQ ID NO: 16), CFHGAT (SEQ ID NO: 17), CVSNP (SEQ ID NO: 18) and CLNGGS (SEQ ID NO: 19)); United States Patent Application Publication No. 2010/0292165 by Clevers et al. (gamma secretase inhibitors including DAPT ((N-[N-(3,5-difluorophenylacetyl)-L-alanyl]-5-phenylglycine tert-butyl ester), dibenzazepine, and a benzodiazepine); United States Patent Application Publication No. 2010/0267801 by Lewis et al.; United States Patent Application Publication No. 2010/0222283 by Susztak et al. (gamma secretase inhibitors including gamma secretase inhibitor I, gamma secretase inhibitor II, gamma secretase inhibitor III, gamma secretase inhibitor IV, gamma secretase inhibitor V, gamma secretase inhibitor VI, gamma secretase inhibitor VII, gamma secretase inhibitor IX, gamma secretase inhibitor X, gamma secretase inhibitor XI, gamma secretase inhibitor XII, gamma secretase inhibitor XIII, gamma secretase inhibitor XIV, gamma secretase inhibitor XVI, gamma secretase inhibitor XVII, gamma secretase inhibitor XIX, gamma secretase inhibitor XX, gamma secretase inhibitor XXI, gamma40 secretase inhibitor I, gamma40 secretase inhibitor II, and isovaleryl-V—V-Sta-A-Sta-OCH₃); and PCT Patent Application Publication No. WO 2012/129353 by Quesnelle et al.

Hsp90 inhibitors include, but are not limited to: IPI-493 (17-amino-17-demethoxygeldanamycin); IPI-504 (retaspimycin hydrochloride); 17-demethoxy-17-(2-propylamino)-geldanamycin; AUY-922 (5-(2,4-dihydroxy-5-isopropylphenyl)-N-ethyl-4-(4-(morpholinomethyl)phenyl)isoxazole-3-carboxamide); elesclomol; alvespimycin (17-demethoxy-17-[[2-(dim ethylamino)ethyl]amino]-geldanamycin hydrochloride); 5′-O-[(4-cyanophenyl)methyl]-8-[[(3,4-dichlorophenyl)methyl]amino]-adenosine; N1-[(3-endo)-8-[5-(cyclopropylcarbonyl)-2-pyridinyl]-8-azabicyclo[3.2.1]oct-3-yl]-2-methyl-5-[[(1R)-1-methylpropyl]amino]-1,4-benzenedicarboxamide; (2,4-dihydroxy-5-isopropylphenyl)(5-((4-methylpiperazin-1-yl)methyl)isoindolin-2-yl)methanone; 4-(6,6-dimethyl-4-oxo-3-(trifluoromethyl)-4,5,6,7-tetrahydroindazol-1-yl)-2-((1r,4r)-4-hydroxycyclohexylamino)benzamide; (1r, 4r)-4-(2-carbamoyl-5-(6,6-dim ethyl-4-oxo-3-(trifluoromethyl)-4,5,6,7-tetrahydroindazol-1-yl)phenylamino)cyclohexyl 2-aminoacetate; 2-amino-4-(2,4-dichloro-5-(2-(pyrrolidin-1-yl)ethoxy)phenyl)-N-ethylthieno[2,3-d]pyrimidine-6-carboxamide; 6-chloro-9-((4-methoxy-3,5-dimethylpyridin-2-yl)methyl)-9H-purin-2-amine; MPC-3100 ((S)-1-(4-(2-(6-amino-84(6-bromobenzo[d][1,3]dioxol-5-yl)thio)-9H-purin-9-yl)ethyl)piperidin-1-yl)-2-hydroxypropan-1-one); CCT-018159 (4-[4-(2,3-dihydro-1,4-benzodioxin-6-yl)-5-methyl-1H-pyrazol-3-yl]-6-ethyl-1,3-benzenediol); CCT-129397 (3-(5-chloro-2,4-dihydroxyphenyl)-N-ethyl-4-(4-m ethoxyphenyl)-1H-pyrazole-5-carboxamide); PU-H71 (6-amino-8-[(6-iodo-1,3-benzodioxol-5-yl)thio]-N-(1-methylethyl)-9H-purine-9-propanamine); SNX-2112 (4-(6,6-dimethyl-4-oxo-3-(trifluoromethyl)-4,5,6,7-tetrahydroindazol-1-yl)-2-((1r,4r)-4-hydroxycyclohexylamino)benzamide; ganetespib; onalespib; XL-888 (2-[[(2R)-butan-2-yl]amino]-4-N-[8-[5-(cyclopropanecarbonyl)pyridin-2-yl]-8-azabicyclo[3.2.1]octan-3-yl]-5-methylbenzene-1,4-dicarboxamide); CU-0305; tanespimycin; macbecin I; macbecin II; 11-O-methyl derivatives of geldanamycin; 17-allylamino-17-demethoxygeldanamycin, 17-(dimethylaminoethylamino)-17-demethoxygeldanamycin; 17-[2-(pyrrolidin-1-yl)ethyl]amino-17-demethoxygeldanamycin; 17-(dimethylaminopropylamino)-17-demethoxygeldanamycin; KF58333 (E isomer); cycloproparadicicol; pochonin D; B-zearalenol; celastrol; gedunin; dacinostat; and romidepsin. Other inhibitors of Hsp90 are known, including: (i) agents that affect post-translational modification, such as acetylation or phosphorylation, of Hsp90; or (ii) recombinant antibodies such as efungumab. Additional inhibitors of Hsp90 are described in the following United States patents and patent applications: U.S. Pat. No. 8,399,426 to Kim et al.; U.S. Pat. No. 8,343,913 to Cowen et al. (geldanamycin, 17-allylamino-17-demethoxygeldanamycin (17-AAG), 17-(desmethoxy)-17-dimethylaminoethylamino-geldanamycin (17-DMAG), radicicol); U.S. Pat. No. 8,329,179 to Ni et al. (17-aminogeldanamycin); U.S. Pat. No. 8,158,638 to Ohsuki et al. (pyrazolopyrimidine derivatives); U.S. Pat. No. 7,129,244 to Kasibhatla et al.; U.S. Pat. No. 6,903,116 to Yokota et al. (benzo-1,3-dioxole); U.S. Pat. No. 6,887,993 to Tian et al. (11-O-methylgeldanamycin compounds); U.S. Pat. No. 6,875,863 to Tian et al.; U.S. Pat. No. 6,872,715 to Santi et al. (benzoquinone amsacrine analogs); U.S. Pat. No. 5,392,566 to Schnur et al. (geldanamycin derivatives); U.S. Pat. No. 5,387,584 to Schnur et al.; U.S. Pat. No. 4,261,989 to Sasaki et al. (geldanamycin derivatives); United States Patent Application Publication No. 2012/0245186 by Blackman et al. (3-(2,4-dihydroxyphenyl)-4-(1-ethyl-indol-4-yl)-5-mercapto-[1,2,4]triazole; 3-(2,4-dihydroxyphenyl)-4-(1-isopropyl-indol-4-yl)-5-mercapto-[1,2,4]triazole; dihydroxyphenyl)-4-(indol-4-yl)-5-mercapto-[1,2,4]triazole; 3-(2,4-dihydroxyphenyl)-4-(1-methoxyethyl-indol-4-yl)-5-mercapto-[1,2,4]triazole; 3-(2,4-dihydroxy-5-ethyl-phenyl)-4-(1-isopropyl-indol-4-yl)-5-mercapto-[1,2,4]triazole; 3-(2,4-dihydroxyphenyl)-4-(1-dimethylcarbamoyl-indol-4-yl)-5-mercapto-[1,2,4]triazole; 3-(2,4-dihydroxy-5-ethyl-phenyl)-4-(1-propyl-indol-4-yl)-5-mercapto-[1,2,4]triazole; 3-(2,4-dihydroxy-5-ethyl-phenyl)-4-(1,2,3-trimethyl-indol-5-yl)-5-mercapto-[1,2,4]triazole; 3-(2,4-dihydroxy-5-ethyl-phenyl)-4-(2,3-dimethyl-indol-5-yl)-5-mercapto-[1,2,4]triazole; 3-(2,4-dihydroxy-5-ethyl-phenyl)-4-(1-acetyl-2,3-dimethyl-indol-5-yl)-5-mercapto-[1,2,4]triazole; 3-(2,4-dihydroxy-5-ethyl-phenyl)-4-(1-isopropyl-7-methoxy-indol-4-yl)-5-mercapto-[1,2,4]triazole; 3-(2,4-dihydroxy-5-ethyl-phenyl)-4-(1-propyl-2,3-dimethyl-indol-5-yl)-5-mercapto-[1,2,4]triazole; 3-(2,4-dihydroxy-5-ethyl-phenyl)-4-(N-methyl-tetrahydrocarbozol-7-yl)-5-mercapto-[1,2,4]triazole; 3-(2,4-dihydroxy-5-ethyl-phenyl)-4-(N-methyl-cyclononan[a]indol-5-yl)-5-mercapto-[1,2,4]triazole; 3-(2,4-dihydroxy-5-ethyl-phenyl)-4-(1-n-butyl-indol-4-yl)-5-mercapto-[1,2,4]triazole; 3-(2,4-dihydroxy-5-ethyl-phenyl)-4-(1-n-pentyl-indol-4-yl)-5-mercapto-[1,2,4]triazole; 3-(2,4-dihydroxy-5-ethyl-phenyl)-4-(1-n-hexyl-indol-4-yl)-5-mercapto-[1,2,4]triazole; 3-(2,4-dihydroxy-5-cyclopropyl-phenyl)-4-(1-(1-methylcyclopropyl)-indol-4-yl)-5-mercapto-[1,2,4]triazole; 3-(2,4-dihydroxy-5-cyclopropyl-phenyl)-4-(1-isopropyl-7-methoxy-indol-4-yl)-5-mercapto-[1,2,4]triazole; 3-(2,4-dihydroxy-5-cyclopropyl-phenyl)-4-(1,2,3-trimethyl-indol-5-yl)-5-mercapto-[1,2,4]triazole; 3-(2,4-dihydroxy-5-ethyl-phenyl)-4-(1-isopropyl-7-methoxy-indol-4-yl)-5-mercapto-[1,2,4]triazole disodium salt; 3-(2,4-dihydroxy-5-tert-butyl-phenyl)-4-(1-isopropyl-7-methoxy-indol-4-yl)-5-mercapto-[1,2,4]triazole; 3-(2,4-dihydroxy-5-cyclopropyl-phenyl)-4-(1-propyl-7-methoxy-indol-4-yl)-5-mercapto-[1,2,4]triazole; 3-(2,4-dihydroxy-5-ethyl-phenyl)-4-(1-methyl-3-ethyl-indol-5-yl)-5-mercapto-[1,2,4]triazole; 3-(2,4-dihydroxy-5-ethyl-phenyl)-4-(1,3-dimethyl-indol-5-yl)-5-mercapto-[1,2,4]triazole; 3-(2,4-dihydroxy-5-isopropyl-phenyl)-4-(1-isopropyl-7-methoxy-indol-4-yl)-5-mercapto-[1,2,4]triazole; 3-(2,4-dihydroxy-5-ethyl-phenyl)-4-(1-methyl-3-isopropyl-indol-5-yl)-5-mercapto-[1,2,4]triazole, 3-(2,4-dihydroxy-5-ethyl-phenyl)-4-(N-ethyl-carbozol-7-yl)-5-mercapto-[1,2,4]triazole; 3-(2,4-dihydroxy-5-ethyl-phenyl)-4-(1-isopropyl-7-hydroxy-indol-4-yl)-5-mercapto-[1,2,4]triazole; 3-(2,4-dihydroxy-5-ethyl-phenyl)-4-(1-isopropyl-7-ethoxy-indol-4-yl)-5-mercapto-[1,2,4]triazole; 3-(2,4-dihydroxy-5-ethyl-phenyl)-4-(1,2-dimethyl-indol-5-yl)-5-mercapto-[1,2,4]triazole; 3-(2,4-dihydroxy-5-ethyl-phenyl)-4-(N-methyl-indol-5-yl)-5-mercapto-[1,2,4]triazole; 3-(2,4-dihydroxy-5-isopropyl-phenyl)-4-(1,3-dimethyl-indol-5-yl)-5-mercapto-[1,2,4]triazole; dihydroxy-5-cyclopropyl-phenyl)-4-(1,3-dimethyl-indol-5-yl)-5-mercapto-[1,2,4]triazole; 3-(2,4-dihydroxy-5-cyclopropyl-phenyl)-4-(1-methyl-indol-5-yl)-5-mercapto-[1,2,4]triazole; 3-(2,4-dihydroxy-5-isopropyl-phenyl)-4-(1H-indol-5-yl)-5-mercapto-[1,2,4]triazole; 3-(2,4-dihydroxy-5-ethyl-phenyl)-4-(1,2-dimethyl-indol-5-yl)-5-mercapto-[1,2,4]triazole; 3-(2,4-dihydroxy-5-isopropyl-phenyl)-4-(1-ethyl-indol-5-yl)-5-mercapto-[1,2,4]triazole; and 3-(2,4-dihydroxy-5-isopropyl-phenyl)-4-(1-propyl-indol-5-yl)-5-mercapto-[1,2,4]triazole); United States Patent Application Publication No. 2012/0022026 by Krawczyk et al. (17-allylamino-17-demethoxygeldanamycin hydroquinone hydrochloride, pochonin, radester, 8-arylsulfanyladenine derivatives, 3,4-d iarylpyrazoleresorcinol derivatives, sheperdin and derivatives thereof, retaspimycin hydrochloride, (−) epigallocatechin-3-gallate, and 4,5-diarylisoxazole derivatives); United States Patent Application Publication No. 2011/0118298 by Fritz et al.; United States Patent Application Publication No. 2010/0093824 by Frydman et al.; United States Patent Application Publication No. 2010/0022635 by Rajewski (N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide; N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide; N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-1H-indole-2-carboxamide; N-(74(2R,3R,4S,5R)-3,4-dihydroxy-5-methoxy-6,6-dimethyl-tetrahydro-2H-pyran-2-yloxy)quinolin-3-yl)-4-methoxy-3-(3-methoxyphenyl)-benzamide; 3-(3′,6-dimethoxybiphenyl-3-ylcarboxamido)-8-methyl-2-oxo-2H-chromen-7-yl propionate; 3-(3′,6-dimethoxybiphenyl-3-ylcarboxamido)-8-methyl-2-oxo-2H-chromen-7-ylcyclopropane carboxylate; and 3-(3′,6-dimethoxybiphenyl-3-ylcarboxamido)-6-methoxy-8-methyl-2-oxo-2H-chromen-7-ylacetate).

Phosphatidylinositide 3-kinase inhibitors include, but are not limited to: wortmannin, demethoxyviridin, LY294002 (2-morpholin-4-yl-8-phenylchromen-4-one), idelalisib, copanlisib, taselisib, buparlisib, duvelisib, alpelisib, umbralisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), dactolisib, CUDC-907 (N-hydroxy-2-(((2-(6-methoxypyridin-3-yl)-4-morpholinothieno[3,2-d]pyrimidin-6-yl)methyl)(methyl)amino)pyrimidine-5-carboxamide), ME-401, IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide), SF1126 ((2S)-2-[[(2S)-3-carboxy-2-[[2-[[2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate), tenalisib, serabilisib, pictilisib, pilaralisib, Palomid 529, GSK1059615 OZ)-5-[(4-pyridin-4-ylquinolin-6-yi)methylidene]-1,3-thiazolidine-2.4-dione), ZSTK474 (4-[4-[2-(difluoromethyl)benzimidazo]-1-yl]-6-morpholin-4-yl-1,3,5-triazin-2-yl]morpholine), PVVT33597 ( ), IC87714 (24(6-amino-9H-purin-9-yl)methyl)-5-methyl-3-o-tolylquinazolin-4(3H)-one), TG100-115 (6,7-bis(3-hydroxyphenyl)pteridine-2,4-diamine), CAL263 ( ), RP6503 ((S)—N-(5-(4-amino-1-(1-(5-fluoro-3-(3-fluorophenyl)-4-oxo-4H-chromen-2-yl) ethyl)-1H-pyrazolo[3,4-d]pyrimidin-3-yl)-2-methoxyphenyl)methanesulfonamide), PI-103 (3-[4-(4-morpholinyl)pyrido[3′,2′:4,5]furo[3,2-d]pyrimidin-2-yl]-phenol), GNE-477 (5-(7-methyl-6-((4-(methylsulfonyl)piperazin-1-yl)methyl)-4-morpholinothieno[3,2-d]pyrimidin-2-yl)pyrimidin-2-amine), and AEZS-136.

Kinase inhibitors are well known in the art. Kinase inhibitors block the phosphorylation of one or more serine, threonine, tyrosine, or in some cases, histidine residues in proteins that are the substrates of kinases. Many kinases regulate cell proliferation and represent targets for chemotherapy. Kinase inhibitors can be either small molecules, monoclonal antibodies, or RNA aptamers. Small-molecule kinase inhibitors include, but are not limited to, afatinib, axitinib, bosutinib, crizotinib, dasatinib, erlotinib, fostamatinib, gefitinib, ibrutinib, lapatinib, lenvatinib, mubritinib, nilotinib, pazopanib, ruxolitinib, sorafenib, sunitinib, SU6656 ((3Z)—N,N-dimethyl-2-oxo-3-(4,5,6,7-tetrahydro-1H-indol-2-ylmethylidene)-2,3-dihydro-1H-indole-5-sulfonamide)), tofacitinib, vandetanib, and vemurafenib. Monoclonal antibody kinase inhibitors include, but are not limited to, bevacizumab, cetuximab, panitumumab, ranibizumab, and trastuzumab. RNA aptamer kinase inhibitors include, but are not limited to, pegaptinib.

Although these methods for preparation and administration are described above with respect to bisantrene dihydrochloride, they are also applicable to other salts of bisantrene and to derivatives or analogs of bisantrene, as well as to prodrugs of bisantrene.

Suitable derivatives and analogs of bisantrene include, but are not limited to the following compounds.

Analogs of bisantrene are disclosed in M. Folini et al., “Remarkable Interference with Telomeric Function by a G-Quadruplex Selective Bisantrene Regioisomer,” Biochem. Pharmacol. 79: 1781-1790 (2010), including compounds of Formulas (II), (III), (IV), (V), (VI), (VII), and (VIII):

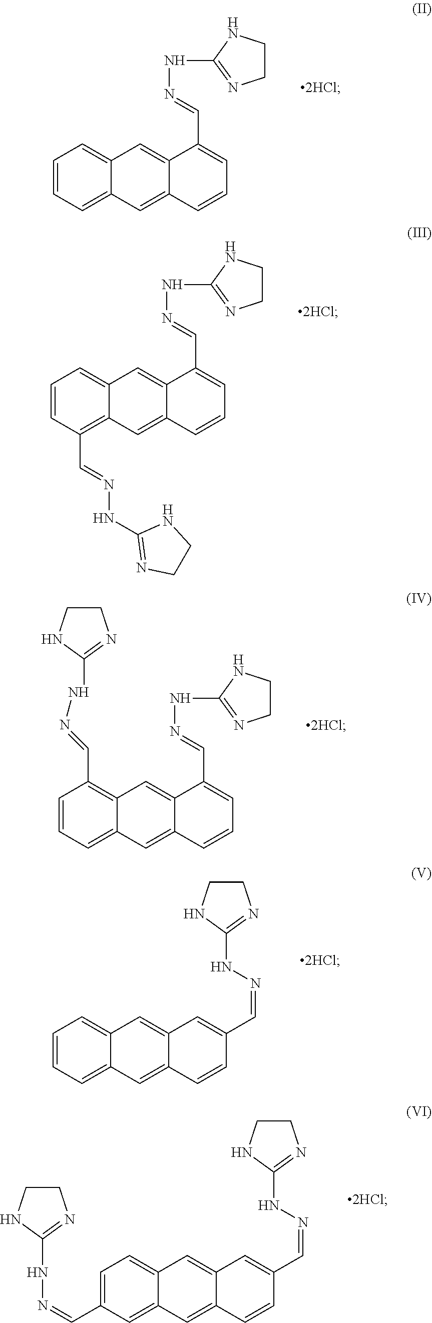

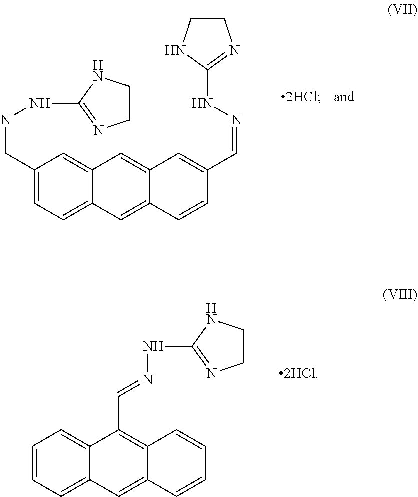 [SMILES: C(=N/NC1=NCCN1)/c1ccc2cc3ccc(CNNC4=NCCN4)cc3cc2c1 ; C(=N\NC1=NCCN1)/c1c2ccccc2cc2ccccc12]

Additional bisantrene analogs have been described in T. P. Wunz et al., “New Antitumor Agents Containing the Anthracene Nucleus,” J. Med. Chem. 30: 1313-1321 (1987), including N,N′-bis[2-(dimethylamino)ethyl]-9,10-anthracene-bis(methylamine) and N,N′-bis(1-ethyl-3-piperidinyl)-9,10-anthracene-bis(methylamine).

Yet another bisantrene analog is the compound known as HL-37 and described in S. Q. Xie et al., “Anti-Tumour Effects of HL-37, a Novel Anthracene Derivative, In-Vivo and In-Vitro,” J. Pharm. Pharmacol. 60: 213-219 (2008). HL-37 is anthracen-9-ylmethylene-[2-methoxyethoxymethylsulfanyl]-5-pyridin-3-yl-[1,2,4]triazol-4-amine and has the structure shown below as Formula (IX):

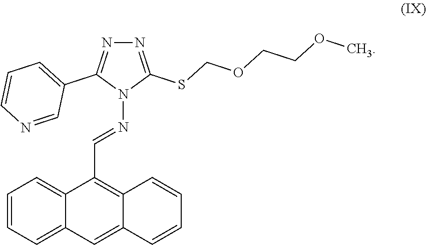

Still other bisantrene analogs are the compounds depicted below as Formulas (X), (XI), (XII), and (XIII):

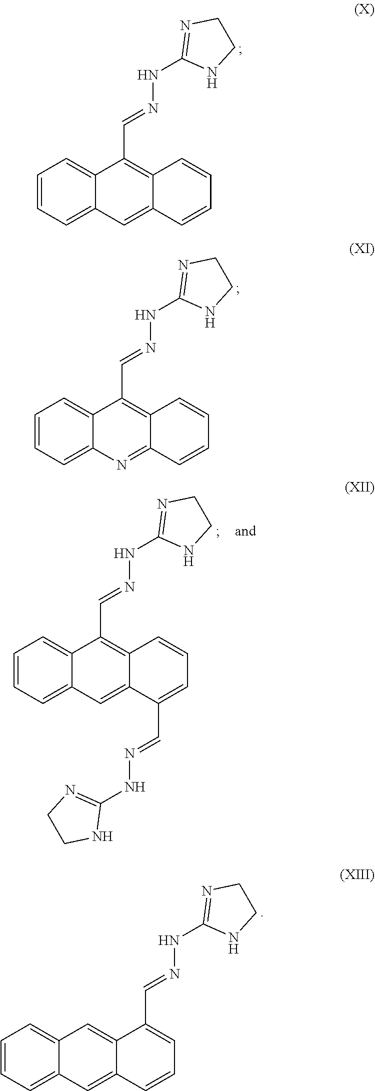

Additional derivatives and analogs of bisantrene include the diphosphoramidic and monophosphoramidic derivatives of bisantrene, disclosed in U.S. Pat. No. 4,900,838 to Murdock and U.S. Pat. No. 5,212,191 to Murdock et al. These compounds are compounds of Formula (XIV):

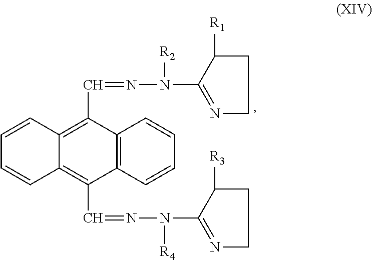

wherein R₁ and R₃ are the same or different and are hydrogen, C₁-C₆ alkyl, —C(O)—R₅, wherein R₅ is hydrogen, C₁-C₆ alkyl, phenyl, mono-substituted phenyl (wherein the substituent can be ortho, meta, or para and is fluoro, nitro, C₁-C₆ alkyl, C₁-C₃ alkoxy, or cyano), pentafluorophenyl, naphthyl, furanyl,

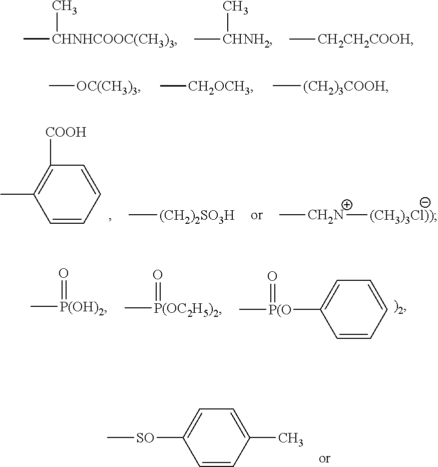

—SO₃H; wherein only one of R₁ and R₃ may be hydrogen or C₁-C₆ alkyl; R₂ and R₄ are the same or different and are: hydrogen, C₁-C₄ alkyl or —C(O)—Re, where R₆ is hydrogen, C₁-C₆ alkyl, phenyl, mono-substituted phenyl (wherein the substituent may be in the ortho, meta, or para position and is fluoro, nitro, C₁-C₆ alkyl, C₁-C₃ alkoxy, or cyano), pentafluorophenyl, naphthyl, furanyl, or —CH₂OCH₃. The compounds can have the schematic structure B(Q)ₙ, wherein B is the residue formed by removal of a hydrogen atom from one or more basic nitrogen atoms of an amine, amidine, guanidine, isourea, isothiourea, or biguanide-containing pharmaceutically active compound, and Q is hydrogen or A, wherein A is

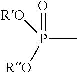

such that R′ and R″ are the same or different and are R (where R is C₁-C₆ alkyl, aryl, aralkyl, heteroalkyl, NC—CH₂CH₂—,

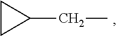

Cl₃C—CH₂—, or R₇OCH₂CH₂—, where R₇ is hydrogen or C₁-C₆ alkyl, hydrogen, or a pharmaceutically acceptable cation or R′ and R″ are linked to form a —CH₂CH₂— group or a

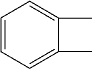

group, and n is an integer representing the number of primary or secondary basic nitrogen atoms in the compound such that at least one Q is A.

Additional bisantrene analogs are disclosed in M. Kozurkova et al., “DNA Binding Properties and Evaluation of Cytotoxic Activity of 9,10-Bis-N-Substituted (Aminomethyl)anthracenes,” Int. J. Biol. Macromol. 41: 415-422 (2007), incorporated herein by this reference. These compounds include 9,10-bis[(2-hydroxyethyl)iminomethyl]anthracene; 9,10-bis{[2-(-2-hydroxyethylamino)ethyl]iminomethyl}anthracene; 9,10-bis{[2-(morpholin-4-yl)ethyl]iminomethyl}anthracene; 9,10-bis[(2-hydroxyethyl)aminomethyl]anthracene; 9,10-bis{[2-(2-hydroxyethylamino)ethyl]aminomethyl}anthracene tetrahydrochloride; 9,10-bis{[2-(piperazin-1-yl)ethyl]aminomethyl}anthracene hexahydrochloride; and 9,10-bis{[2-(morpholin-4-yl)ethyl]aminomethyl}anthracene tetrahydrochloride.

Other analogs and derivatives are known in the art, including derivatives and salt forms of the compounds described above. In particular, positively-charged bisantrene derivatives and analogs can form salts such as, but not limited to, salts including sulfates, pyrosulfates, bisulfates, sulfites, bisulfites, phosphates, monohydrogenphosphates, dihydrogenphosphates, metaphosphates, pyrophosphates, bromides, iodides, acetates, propionates, decanoates, caprylates, acrylates, formates, isobutyrates, caproates, heptanoates, propionates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methanesulfonates, propanesulfonates, naphthalene-1-sulfonates, naphthalene-2-sulfonates, and mandelates, as well as with other negatively-charged counterions.

In another alternative, bisantrene dihydrochloride or a derivative or analog thereof can also be formulated and administered as a prodrug. As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24), incorporated herein by this reference. A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987), both incorporated herein by this reference. Exemplary advantages of a prodrug can include, but are not limited to, its physical properties, such as enhanced water solubility for parenteral administration at physiological pH compared to the parent compound, enhanced absorption from the digestive tract, or enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, γ-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y′ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Jarvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796, incorporated herein by this reference.

### Advantages of the Invention

The present invention provides compositions and improved methods for preparing and delivering bisantrene formulations that results in improved bioavailability and fewer side effects resulting from administration of the bisantrene; in particular, administration of the formulations of the present invention reduce the frequency and severity of phlebitis. Compositions according to the present invention can be administered to treat a range of malignancies, and can be used together with other anti-neoplastic drugs; they can also be used to treat other diseases and conditions. In particular, bisantrene has therapeutic activity of the anthracycline class without the common dose limiting toxicities of congestive heart disease and multi-drug resistance which limits the use of all other drugs in the class. By manufacturing and administering bisantrene in the manner presented herein the major dose limiting toxicity associated with bisantrene is significantly reduced allowing for broader use, better drug delivery and uptake, and better therapeutic outcomes, especially in patients with disease who have reached the end of the safe limits of their anthracycline exposure.

Compositions according to the present invention possess industrial applicability for treatment of diseases and conditions, including, but not limited to, malignancies. Methods for preparing compositions according to the present invention possess industrial applicability as methods for preparation of a pharmaceutically useful composition. Methods for administering compositions according to the present invention possess industrial applicability for the preparation of a medicament for the treatment of a number of diseases and conditions.

Where methods are referred to, the methods of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug or new formulations of an existing drug.

The inventions illustratively described herein can suitably be practiced in the absence of any element or elements, limitation or limitations, not specifically disclosed herein. Thus, for example, the terms “comprising,” “including,” “containing,” and similar language shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. Moreover, when the term “comprising” is used herein as a transitional phrase in claims, the term “comprising” also includes both “consisting essentially of” and “consisting of” unless the narrower terms are specifically excluded.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of skill in the art upon reviewing the above description. The scope of the invention should, therefore, be determined not with reference to the above description, but should instead be determined with reference to the appended claims, along with the full scope of equivalents to which such claims are entitled. The disclosures of all articles and references, including patents and patent publications, are incorporated herein by reference.

1. A method for preparing bisantrene dihydrochloride units for delivery to a patient in need of treatment with bisantrene dihydrochloride comprising the steps of:
(a) preparing an initial stock solution of bisantrene dihydrochloride;
(b) filtering the initial stock solution of bisantrene dihydrochloride;
(c) aliquoting the initial stock solution of bisantrene dihydrochloride into vials; and
(d) lyophilizing the aliquoted stock solution in the vials, wherein the initial stock solution is prepared at a temperature of about 20° C. to about 25° C. or at a temperature of about 4° C.

2. The method of claim 1 wherein the initial stock solution of bisantrene dihydrochloride is prepared in sterile water for injection.

3. The method of claim 1 wherein the initial stock solution is prepared at a temperature of about 20° C. to about 25° C. or about 4° C.

4. (canceled)

5. The method of claim 1 wherein the initial stock solution is prepared at a concentration of between about 25 mg/mL and about 40 mg/mL.

6. (canceled)

7. The method of claim 1 wherein the initial stock solution is filtered through 1 to 3 filters.

8. The method of claim 7 wherein the initial stock solution is filtered through 1 filter.

9. The method of claim 8 wherein the filter has a filtration cutoff of about 0.2 μm.

10. The method of claim 7 wherein the initial stock solution is filtered through 2 filters.

11. The method of claim 10 wherein the first filter has a filtration cutoff of about 1-2 μm.

12. The method of claim 10 wherein the second filter has a filtration cutoff of about 0.2 μm.

13. The method of claim 7 wherein the initial stock solution is filtered through 3 filters.

14. The method of claim 13 wherein the first filter has a filtration cutoff of about 4-6 μm.

15. The method of claim 13 wherein the second filter has a filtration cutoff of about 1-2 μm.

16. The method of claim 13 wherein the third filter has a filtration cutoff of about 0.2 μm.

17. The method of claim 1 wherein the vials are plastic vials or glass vials, wherein the glass vials are optionally silanized.

18.-20. (canceled)

21. The method of claim 17 wherein, when the vials are plastic vials, wherein the plastic is selected from the group consisting of cyclic olefin polymer (COP) plastic, cyclic olefin copolymer (COC) plastic, high-density polyethylene plastic, and high-density non-nucleated polypropylene plastic.

22. The method of claim 1 wherein the volume of stock solution aliquoted into each vial is consistent with delivery of about 295 mg of bisantrene dihydrochloride into each vial.

23.-27. (canceled)

28. A method for delivering bisantrene dihydrochloride units to a patient in need of treatment with bisantrene dihydrochloride comprising the steps of:
(a) reconstituting the contents of a bisantrene dihydrochloride unit vial with sterile water;
(b) filtering the reconstituted bisantrene dihydrochloride into a suitable intravenous infusion vehicle; and
(c) infusing into a patient a therapeutic volume of the bisantrene dihydrochloride-infusion vehicle formulation.

29. The method of claim 28 wherein the bisantrene dihydrochloride units comprise about 295 mg of lyophilized bisantrene dihydrochloride.

30. The method of claim 28 wherein the contents of a bisantrene dihydrochloride unit vial are reconstituted with about 9 mL to about 11 mL of sterile water.

31. (canceled)

32. The method of claim 28 wherein the filter is a sterile syringe filter and wherein the sterile syringe filter has a filtration cutoff in a range of from about 0.15 μm to about 0.25 μm.

33.-35. (canceled)

36. The method of claim 28 wherein the filter is washed into the intravenous infusion vehicle with an additional volume of sterile water.

37.-38. (canceled)

39. The method of claim 28 wherein the suitable intravenous infusion vehicle is 5% dextrose in water.

40.-45. (canceled)

46. The method of claim 28 wherein the bisantrene dihydrochloride-infusion vehicle formulation is infused into a patient through an intravenous infusion set containing an in-line filter.

47.-49. (canceled)

50. The method of claim 28 wherein the duration of the infusion is from about 1.5 hours to about 2.5 hours.

51.-52. (canceled)

53. The method of claim 28 wherein the dosage received by the patient is from about 200 mg/m² to about 300 mg/m² body surface area.

54.-55. (canceled)

56. The method of claim 28 wherein the method further comprises the step of administering to a patient a therapeutically effective quantity of an additional therapeutic agent.

57. The method of claim 28 wherein the bisantrene dihydrochloride is administered to the patient to treat a malignancy selected from the group consisting of: breast cancer, acute myelocytic leukemia, acute lymphocytic leukemia of childhood, myelodysplastic syndrome, chronic myelocytic leukemia, chronic lymphocytic leukemia, Hodgkin's lymphoma, non-Hodgkin's lymphoma, mycosis fungoides, prostate cancer, lung small-cell carcinoma, lung non-small cell carcinoma, glioblastoma, a malignancy characterized by overexpressed topoisomerase II, a malignancy characterized by overexpressed and/or mutated EGFR, ovarian cancer, renal cancer, melanoma, gastric cancer, adrenal cancer, head and neck cancer, hepatocellular cancer, hypernephroma, bladder cancer, myeloma, and localized polyp stage colon cancer.

58. The method of claim 57 wherein the malignancy is selected from the group consisting of:
(A) breast cancer, wherein the method further comprises the step of administering to the patient a therapeutically effective quantity of an additional therapeutic agent, and wherein the additional therapeutic agent is selected from the group consisting of tamoxifen, anastrozole, letrozole, cyclophosphamide, docetaxel, paclitaxel, methotrexate, fluorouracil, and trastuzumab;
(b) acute myelocytic leukemia, wherein the method further comprises the step of administering to the patient a therapeutically effective quantity of an additional therapeutic agent, and wherein the additional therapeutic agent is selected from the group consisting of cytarabine, fludarabine, all-trans-retinoic acid, interleukin-2, and arsenic trioxide;
(c) myelodysplastic syndrome, wherein the method further comprises the step of administering to the patient a therapeutically effective quantity of an additional therapeutic agent, and wherein the additional therapeutic agent is selected from the group consisting of 5-azacytidine, decitabine, and lenalidomide;
(d) chronic myelocytic leukemia, wherein the method further comprises the step of administering to the patient a therapeutically effective quantity of an additional therapeutic agent, and wherein the additional therapeutic agent is selected from the group consisting of: cytarabine; hydroxyurea; an alkylating agent selected from the group consisting of melphalan, chlorambucil, cyclophosphamide, mechlorethamine, uramustine, ifosfamide, bendamustine, carmustine, lomustine, streptozotocin, busulfan, procarbazine, altretamine, dacarbazine, temozolomide, and mitozolomide; interferon alfa 2b; a steroid selected from the group consisting of prednisone and prednisolone; and a Bcr-Abl tyrosine kinase inhibitor selected from the group consisting of imatinib, dasatinib, bosutinib, and radotinib;
(e) chronic lymphocytic leukemia, wherein the method further comprises the step of administering to the patient a therapeutically effective quantity of an additional therapeutic agent, and wherein the additional therapeutic agent is selected from the group consisting of fludarabine, cyclophosphamide, rituximab, vincristine, prednisolone, bendamustine, alemtuzumab, ofatumumab, obinutuzumab, ibrutinib, idelalisib, and venetoclax;
(f) Hodgkin's lymphoma, wherein the method further comprises the step of administering to the patient a therapeutically effective quantity of an additional therapeutic agent, and wherein the additional therapeutic agent is selected from the group consisting of mechlorethamine, vincristine, prednisone, procarbazine, bleomycin, vinblastine, dacarbazine, etoposide, and cyclophosphamide;
(g) non-Hodgkin's lymphoma, wherein the method further comprises the step of administering to the patient a therapeutically effective quantity of an additional therapeutic agent, and wherein the additional therapeutic agent is selected from the group consisting of cyclophosphamide, vincristine, and prednisone;
(h) mycosis fungoides, wherein the method further comprises the step of administering to the patient a therapeutically effective quantity of an additional therapeutic agent, and wherein the additional therapeutic agent is selected from the group consisting of a corticosteroid, etretinate, arotinoid, acitretin, isotretinoin, bexarotene, carmustine, methotrexate, vorinostat, interferon α, denileukin diftitox, mechlorethamine, depsipeptide, panobinostat, belinostat, alemtuzumab, zanolimumab, cyclophosphamide, chlorambucil, etoposide, dexamethasone, doxorubicin, bleomycin, and vinblastine;
(i) prostate cancer, wherein the method further comprises the step of administering to the patient a therapeutically effective quantity of an additional therapeutic agent, and wherein the additional therapeutic agent is selected from the group consisting of temozolomide, docetaxel, cabazitaxel, bevacizumab, thalidomide, prednisone, sipuleucel-T, abiraterone, and enzalutamide,
(j) lung small-cell carcinoma, wherein the method further comprises the step of administering to the patient a therapeutically effective quantity of an additional therapeutic agent, and wherein the additional therapeutic agent is selected from the group consisting of cyclophosphamide, cisplatin, etoposide, vincristine, paclitaxel, and carboplatin,
(k) lung non-small-cell carcinoma, wherein the method further comprises the step of administering to the patient a therapeutically effective quantity of an additional therapeutic agent, and wherein the additional therapeutic agent is selected from the group consisting of cisplatin, erlotinib, gefitinib, afatinib, crizotinib, bevacizumab, carboplatin, paclitaxel, nivolumab, and embrolizumab,
(l) glioblastoma, wherein the method further comprises the step of administering to the patient a therapeutically effective quantity of an additional therapeutic agent, and wherein the additional therapeutic agent is selected from the group consisting of temozolomide and bevacizumab,
(m) a malignancy characterized by overexpressed topoisomerase II, wherein the method further comprises the step of administering to the patient a therapeutically effective quantity of an additional therapeutic agent, and wherein the additional therapeutic agent is selected from the group consisting of etoposide, teniposide, doxorubicin, daunorubicin, mitoxantrone, amsacrine, ellipticine, aurintricarboxylic acid, and HU-331 (3-hydroxy-2-[(1R)-6-isopropenyl-3-methyl-cyclohex-2-en-1-yl]-5-pentyl-1,4-benzoquinone), a malignancy characterized by overexpressed and/or mutated EGFR,
(n) wherein the method further comprises the step of administering to the patient a therapeutically effective quantity of an additional therapeutic agent, and wherein the additional therapeutic agent is selected from the group consisting of gefitinib, erlotinib, afatinib, brigatinib, icotinib, cetuximab, osimertinib, panitumumab, zalutumumab, nimotuzumab, matuzumab, and lapatinib,
(o) ovarian cancer, wherein the method further comprises the step of administering to the patient a therapeutically effective quantity of an additional therapeutic agent, and wherein the additional therapeutic agent is selected from the group consisting of: a platinum-containing antineoplastic drug selected from the group consisting of cisplatin, carboplatin, oxaliplatin, nedaplatin, triplatin, phenanthriplatin, picoplatin, and satraplatin; paclitaxel; topotecan; gemcitabine; etoposide; and bleomycin;
(p) renal cancer, wherein the method further comprises the step of administering to the patient a therapeutically effective quantity of an additional therapeutic agent, and wherein the additional therapeutic agent is selected from the group consisting of everolimus, torisel, nexavar, sunitinib, axitinib, inferferon, interleukin-2, pazopanib, sorafenib, nivolumab, cabozanitib, and levanitib;
(q) melanoma, wherein the method further comprises the step of administering to the patient a therapeutically effective quantity of an additional therapeutic agent, and wherein the additional therapeutic agent is selected from the group consisting of temozolomide, dacarbazine, interferon, interleukin-2, ipilimumab, pembrolizumab, nivolumab, vemurafenib, dabrafenib, and trametinib;
(r) gastric cancer, wherein the method further comprises the step of administering to the patient a therapeutically effective quantity of an additional therapeutic agent, and wherein the additional therapeutic agent is selected from the group consisting of 5-fluorouracil, capecitabine, carmustine, semustine, doxorubicin, mitomycin C, cisplatin, taxotere, and trastuzumab;
(s) adrenal cancer, wherein the method further comprises the step of administering to the patient a therapeutically effective quantity of an additional therapeutic agent, and wherein the additional therapeutic agent is selected from the group consisting of mitotane, cisplatin, etoposide, and streptozotocin;
(t) head and neck cancer, wherein the method further comprises the step of administering to the patient a therapeutically effective quantity of an additional therapeutic agent, and wherein the additional therapeutic agent is selected from the group consisting of paclitaxel, carboplatin, cetuximab, docetaxel, cisplatin, and 5-fluorouracil;
(u) hepatocellular cancer, wherein the method further comprises the step of administering to the patient a therapeutically effective quantity of an additional therapeutic agent, and wherein the additional therapeutic agent is selected from the group consisting of tamoxifen, octreoside, synthetic retinoids, cisplatin, 5-fluorouracil, interferon, taxol, and sorafenib;
(v) hypernephroma, wherein the method further comprises the step of administering to the patient a therapeutically effective quantity of an additional therapeutic agent, and wherein the additional therapeutic agent is selected from the group consisting of nivolumab, everolimus, sorafenib, axitinib, lenvatinib, temsirolimus, sunitinib, pazopanib, interleukin-2, cabozanitib, bevacizumab, interferon α, ipilimumab, atezolizumab, varilumab, durvalumab, tremelimumab, and avelumab,
(w) bladder cancer, wherein the method further comprises the step of administering to the patient a therapeutically effective quantity of an additional therapeutic agent, and wherein the additional therapeutic agent is selected from the group consisting of cisplatin, 5-fluorouracil, mitomycin C, gemcitabine, methotrexate, vinblastine, carboplatin, paclitaxel, and emetrexed,
(x) myeloma, wherein the method further comprises the step of administering to the patient a therapeutically effective quantity of an additional therapeutic agent, and wherein the additional therapeutic agent is selected from the group consisting of bortezomib, lenalidomide, dexamethasone, melphalan, prednisone, thalidomide, and cyclophosphamide, and
(y) localized poly stage colon cancer, wherein the method further comprises the step of administering to the patient a therapeutically effective quantity of an additional therapeutic agent, and wherein the additional therapeutic agent is selected from the group consisting of wherein the additional therapeutic agent is selected from the group consisting of tegafur/uracil, capecitabine, 5-fluorouracil, oxaliplatin, irinotecan, bevacizumab, cetuximab, panitumumab, and folinic acid.

59.-116. (canceled)

117. The method of claim 56 wherein the additional agent is selected from the group consisting of: an agent inducing immunoactivity; an agent inducing macrophage activation; a cytokine; an agent inhibiting telomerase; an agent inhibiting survivin; an agent inhibiting methylation or modulating demethylation; an adjuvant; an antibody; an innate or adaptive immune stimulator; a checkpoint inhibitor; a mTOR antagonist; an Akt inhibitor; a notch inhibitor; an Hsp90 inhibitor; a phosphatidylinositide 3-kinase inhibitor; a kinase inhibitor; taxane; and taxol.

118. The method of claim 117 wherein the additional agent is selected from the group consisting of:
(A) a cytokine, wherein the cytokine is selected from the group consisting of interleukin-1, interleukin-2, interleukin-4, interleukin-5, interleukin-6, interferon-γ, TGF-β, interleukin-3, interleukin-7, GMCSF, MIP-1a, MIP-1b, MCP-1, RANTES, interleukin-8, lymphotactin, fractalkine, interleukin-10, interleukin-13, interferon-α, and interferon-β;
(b) a telomerase inhibitor, wherein the telomerase inhibitor is selected from the group consisting of 7-deaza-2′-deoxyguanosine, antisense oligonucleotides, imetelstat, BPPA (2,6-bis(3-piperidinopropionamido)anthraquinone), (−)-epigallocatechin gallate, H-7 (2,6-bis(3-piperidinopropionamido)anthraquinone), 13-rubromycin, and BIBR1532 (2-[[(2E)-3-(2-naphthalenyl)-1-oxo-2-butenyl1-yl]amino]benzoic acid);
(c) an inhibitor of survivin, wherein the inhibitor of survivin is selected from the group consisting of: antisense oligonucleotides; YM155 (septantronium bromide); 5-aminoimidazole-4-carboxamide-1-β-D-furanoside (AICAR); arctigenin; cephalochromin; FL118 (7-ethyl-7-hydroxy-10H-[1,3]dioxolo[4,5-g]pyrano[3′,4′:6,7]indolizino[1,2-b]quinoline-8,11(7H,13H)-dione); flavopiridol; KPT-185 (isopropyl (Z)-3-(3-(3-methoxy-5-(trifluoromethyl)phenyl)-1H-1,2,4-triazol-1-yl)acrylate); lapatinib; MK-2206 (8-(4-(1-aminocyclobutyl)phenyl)-9-phenyl-[1,2,4]triazolo[3,4-f][1,6]naphthyridin-3(2H)-one); panepoxydone; piperine; purvalanol A; shepherdin; terameprocol; UC112 (5-[(phenylmethoxy)methyl]-7-(1-pyrrolidinylmethyl)-8-quinolinol); NSC80467 (2-methyl-1-(2-methylpropyl)-3-[2-(4-nitrophenyl)-2-oxoethyl]benzo[f]benzimidazol-3-ium-4,9-dione bromide); SPC3042 (a locked antisense nucleic acid designed as an antisense 16-mer LNA gapmer; NU6140 (4-(6-cyclohexylmethoxy-9H-purin-2-ylamino)-N,N-diethylbenzamide); toxoflavin; gambogic acid, LLP-3 (4-(3,5-bis(benzyloxy)phenyl)-6-(5-chloro-2-hydroxyphenyl)-2-oxo-1,2-dihydropyridine-3-carbonitrile); gataparsen; (6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide; 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate; tetra-O-methyl-nordihydroguaiaretic acid; 1,4-bis[3,4-bis[3-(piperidin-1-yl)propoxy]phenyl]-butane; tetra-substituted nordihydroguaiaretic acid derivatives via ether bonds or carbamate bonds; tetraglycinyl nordihydroguaiaretic acid; LY2181308; dichloroacetic acid; and ICG-001 ((6S,9aS)-6-(4-hydroxybenzyl)-N-benzyl-8-(naphthalen-1-ylmethyl)-4,7-dioxo-hexahydro-2H-pyrazino[1,2-a]pyrimidine-1(6H)-carboxamide);
(d) an agent inhibiting methylation, wherein the agent inhibiting methylation is selected from the group consisting of 5′-azacytidine, 5-aza-2′-deoxycytidine, zebularine, L-methionine, apicidine, hydralazine, procainamide, and antisense oligonucleotides directed against mRNA for DNA methyltransferase;
(e) an agent modulating demethylation, wherein the agent modulating demethylation is an inhibitor of histone deacetylase selected from the group consisting of N-hydroxy-3-[4-[[(2-hydroxyethyl)[2-(1H-indol-3-yl)ethyl]-amino]methyl]phenyl]-2E-2-propenamide, suberoylanilide hydroxamic acid, 4-(2-amino-phenylcarbamoyl)-benzyl]-carbamic acid pyridine-3-ylmethyl ester and derivatives thereof, butyric acid, pyroxamide, trichostatin A, oxamflatin, apicidin, depsipeptide, depudecin, trapoxin, HC toxin, and sodium phenylbutyrate;
(f) an adjuvant, wherein the adjuvant is selected from the group consisting of GM-CSF, poly-ICLC (carboxymethylcellulose, polyinosinic-polycytidylic acid, and poly L-lysine), nanoparticles, microparticles, aluminum salts, squalene, QS-21 (a plant extract from Quillaja saponaria containing water-soluble triterpene glycosides), virosomes, IL-2, IL-7, IL-21, and type 1 interferons;
(g) a checkpoint inhibitor, wherein the checkpoint inhibitor is selected from the group consisting of ipilimumab, nivolumab, pembrolizumab, atezolizumab, avelumab, durvalumab, and spartalizumab;
(h) a mTOR inhibitor, wherein the mTOR inhibitor is selected from the group consisting of: sirolimus; temsirolimus; everolimus; rapamune; ridaforolimus; AP23573 (deforolimus); CCI-779 (rapamycin 42-ester with 3-hydroxy-2-(hydroxymethyl)-2-methylpropionic acid); AZD8055 ((5-(2,4-bis((S)-3-methylmorpholino)pyrido[2,3-d]pyrimidin-7-yl)-2-methoxyphenyl)methanol), PKI-587 (1-(4-(4-(dimethylamino)piperidine-1-carbonyl)phenyl)-3-(4-(4,6-dimorpholino-1,3,5-triazin-2-yl)phenyl)urea), NVP-BEZ235 (2-methyl-2-{4-[3-methyl-2-oxo-8-(quinolin-3-yl)-2,3-dihydro-1H-imidazo[4,5-c]quinolin-1-yl]phenyl}propanenitrile), LY294002 ((2-(4-morpholinyl)-8-phenyl-4H-1-benzopyran-4-one), 40-O-(2-hydroxyethyl)-rapamycin, ABT578 (zotarolimus), biolimus-7, biolimus-9, AP23675, AP23841, TAFA-93, 42-O-(methyl-D-glucosylcarbonyl)rapamycin, 42-O-[2-(methyl-D-glucosylcarbonyloxy)ethyl]rapamycin, 31-O-(methyl-D-glucosylcarbonyl)rapamycin, 42-O-(2-hydroxy ethyl)-31-O-(methyl-D-glucosylcarbonyl)rapamycin, 42-O-(2-O-methyl-D-fructosylcarbonyl)rapamycin, 42-O-[2-(2-O-methyl-D-fructosylcarbonyloxy)ethyl]rapamycin, 42-O-(2-O-methyl-L-fructosylcarbonyl)rapamycin, 42-O-[2-(2-O-methyl-L-fructosylcarbonyloxy)ethyl]rapamycin, 31-O-(2-O-methyl-D-fructosylcarbonyl)rapamycin, 42-O-(2-hydroxyethyl)-31-O-(2-O-methyl-D-fructosylcarbonyl)rapamycin; 31-O-[2-O-methyl-L-fructosylcarbonyl)rapamycin; 42-O-(2-hydroxyethyl)-31-O-(2-O-methyl-L-fructosylcarbonyl)rapamycin, 42-O-(D-allosylcarbonyl)rapamycin, 42-O-[2-(D-allosylcarbonyloxy)ethyl]rapamycin, 42-O-(L-allosylcarbonyl)rapamycin, 42-O-[2-(L-allosylcarbonyloxy)ethyl]rapamycin, 31-O-(D-allosylcarbonyl)rapamycin, 42-O-(2-hydroxyethyl)-31-O-(D-allosylcarbonyl)rapamycin, 31-O-(L-allosylcarbonyl)rapamycin, 42-O-(2-hydroxy ethyl)-31-O-(L-allosylcarbonyl)rapamycin, 42-O-(D- fructosylcarbonyl)rapamycin, 42-O-[2-(D-fructosylcarbonyloxy)ethyl]rapamycin, 42-O-(L-fructosylcarbonyl)rapamycin, 42-O-(2-fructosylcarbonyloxy)ethyl]rapamycin, 31-O-(D-fructosylcarbonyl)rapamycin, 42-O-(2-hydroxyethyl)-31-O-(D-fructosylcarbonyl)rapamycin, 31-O-(L-fructosylcarbonyl)rapamycin, 42-O-(2-hydroxyethyl)-31-O-(D-fructosylcarbonyl)rapamycin, 42-O-(D-fucitolylcarbonyl)rapamycin, 42-O-[2-(D-fucitolylcarbonyloxy)ethyl]rapamycin, 42-O-(L-fucitolylcarbonyl)rapamycin, 42-O-[2-(L-fucitolylcarbonyloxy)ethyl]rapamycin, 31-O-(D-fucitolylcarbonyl)rapamycin, 42-O-(2-hydroxyethyl)-31-O-(D-fucitolylcarbonyl)rapamycin, 31-O-(L-fucitolylcarbonyl)rapamycin, 42-O-(2-hydroxyethyl)-31-O(L-fucitolylcarbonyl)rapamycin, 42-O-(D-glucalylcarbonyl)rapamycin, 42-O-[2-(D-glucalylcarbonyloxy)ethyl]rapamycin, 42-O-(D-glucosylcarbonyl)rapamycin, 42-O-[2-(D-glucosylcarbonyloxy)ethyl]rapamycin, glucosylcarbonyl)rapamycin, 42-O-[2-(L-glucosylcarbonyloxy)ethyl]rapamycin, 31-O-(D-glucalylcarbonyl)rapamycin, 42-O-(2-hydroxyethyl)-31-O-(D-glucalylcarbonyl)rapamycin, 31-O-(D-glucosylcarbonyl)rapamycin; 42-O-(2-hydroxyethyl)-31-O-(D-glucosylcarbonyl)rapamycin; 31-O(L-glucosylcarbonyl)rapamycin; 42-O-(2-hydroxyethyl)-31-O-(L-glucosylcarbonyl)rapamycin; 42-O(L-sorbosylcarbonyl)rapamycin; 42-O-(D-sorbosylcarbonyl)rapamycin; 31-O(L-sorbosylcarbonyl)rapamycin; 31-O-(D-sorbosylcarbonyl)rapamycin; 42-O-[2-(L-sorbosylcarbonyloxy)ethyl]rapamycin; 42-O-[2-(D-sorbosylcarbonyloxy)ethyl]rapamycin; 42-O-(2-hydroxyethyl)-31-O-(D-sorbosylcarbonyl)rapamycin; 42-O-(2-hydroxyethyl)-31-O(L-sorbosylcarbonyl)rapamycin; 42-O-(D-lactalylcarbonyl)rapamycin; 42-O-[2-(D-lactalylcarbonyloxy)ethyl]rapamycin; 31-O-(D-lactalylcarbonyl)rapamycin; 42-O-(2-hydroxyethyl)-31-O-(D-lactalylcarbonyl)rapamycin; 42-O-(D-sucrosylcarbonyl)rapamycin; 42-O-[2-(D-sucrosylcarbonyloxy)ethyl]rapamycin; 31-O-(D-sucrosylcarbonyl)rapamycin; 42-O-(2-hydroxyethyl)-31-O-(D-sucrosylcarbonyl)rapamycin; 42-O-(D-gentobiosylcarbonyl)rapamycin; 42-O-[2-(D-gentobiosylcarbonyloxy)ethyl]rapamycin; 31-O-(D-gentobiosylcarbonyl)rapamycin; 42-O-(2-hydroxyethyl)-31-O-(D-gentobiosylcarbonyl)rapamycin; 42-O-(D-cellobiosylcarbonyl)rapamycin; 42-O-[2-(D-cellobiosylcarbonyloxy)ethyl]rapamycin; 31-O-(D-cellobiosylcarbonyl)rapamycin; 42-O-(2-hydroxyethyl)-31-O-(D-cellobiosylcarbonyl)rapamycin; 42-O-(D-turanosylcarbonyl)rapamycin; 42-O-[2-(D-turanosylcarbonyloxy)ethyl]rapamycin; 31-O-(D-turanosylcarbonyl)rapamycin; 42-O-(2-hydroxyethyl)-31-O-(D-turanosylcarbonyl)rapamycin; 42-O-(D-palatinosylcarbonyl)rapamycin; 42-O-[2-(D-palatinosylcarbonyloxy)ethyl]rapamycin; 31-O-(p-palatinosylcarbonyl)rapamycin; 42-O-(2-hydroxyethyl)-31-O-(D-palatinosylcarbonyl)rapamycin; 42-O-(D-isomaltosylcarbonyl)rapamycin; 42-O-[2-(D-isomaltosylcarbonyloxy)ethyl]rapamycin; 31-O-(D-isomaltosylcarbonyl)rapamycin; 42-O-(2-hydroxyethyl)-31-O-(D-isomaltosylcarbonyl)rapamycin; 42-O-(D-maltulosylcarbonyl)rapamycin; 42-O-[2-(D-maltulosylcarbonyloxy)ethyl]rapamycin; 42-O-(D-maltosylcarbonyl)rapamycin; 42-O-[2-(D-maltosylcarbonyloxy)ethyl]rapamycin; 31-O-(D-maltulosylcarbonyl)rapamycin; 42-O-(2-hydroxyethyl)-31-O-(D-maltulosylcarbonyl)rapamycin; 31-O-(D-maltosylcarbonyl)rapamycin; 42-O-(2-hydroxyethyl)-31-O-(D-maltosylcarbonyl)rapamycin; 42-O-(D-lactosylcarbonyl)rapamycin; 42-O-[2-(D-lactosylcarbonyloxy)ethyl]rapamycin; 31-O-(methyl-D-lactosylcarbonyl)rapamycin; 42-O-(2-hydroxyethyl)-31-O-(methyl-D-lactosylcarbonyl)rapamycin; 42-O-(D-melibiosylcarbonyl)rapamycin, 31-O-(D-melibiosylcarbonyl)rapamycin, 42-O-(2-hydroxyethyl)-31-O-(D-melibiosylcarbonyl)rapamycin, 42-O-(D-leucrosylcarbonyl)rapamycin, 42-O-[2-(D-leucrosylcarbonyloxy)ethyl]rapamycin; 31-O-(D-leucrosylcarbonyl)rapamycin, 42-O-(2-hydroxyethyl)-31-O-(D-leucrosylcarbonyl)rapamycin, 42-O-(D-raffinosylcarbonyl)rapamycin, 42-O-[2-(D-raffinosylcarbonyloxy)ethyl]rapamycin, 31-O-(D-raffinosylcarbonyl)rapamycin, 42-O-(2-hydroxyethyl)-31-O-(D-raffinosylcarbonyl)rapamycin, 42-O-(D-isomaltotriosylcarbonyl)rapamycin; 42-O-[2-(D-isomaltosylcarbonyloxy)ethyl]rapamycin, 31-O-(D-isomaltotriosylcarbonyl)rapamycin, 42-O-(2-hydroxyethyl)-31-O-(D-isomaltotriosylcarbonyl)rapamycin; 42-O-(D-cellotetraosylcarbonyl)rapamycin, 42-O-[2-(D-cellotetraosylcarbonyloxy)ethyl]rapamycin; 31-O-(D-cellotetraosylcarbonyl)rapamycin, 42-O-(2-hydroxyethyl)-31-O-(D-cell otetraosyl carbonyl)rapamycin; 42-O-(valiolylcarbonyl)rapamycin, 42-O-[2-(D-valiolylcarbonyloxy)ethyl]rapamycin, 31-O-(valiolylcarbonyl)rapamycin, 42-O-(2-hydroxyethyl)-31-O-(valiolylcarbonyl)rapamycin, 42-O-(valiolonylcarbonyl)rapamycin, 42-O-[2-(D-valiolonylcarbonyloxy)ethyl]rapamycin, 31-O-(valiolonylcarbonyl)rapamycin, 42-O-(2-hydroxyethyl)-31-O-(valiolonylcarbonyl)rapamycin, 42-O-(valienolylcarbonyl)rapamycin, 42-O-[2-(D-valienolylcarbonyloxy)ethyl]rapamycin, 31-O-(valienolylcarbonyl)rapamycin, 42-O-(2-hydroxyethyl)-31-O-(valienolylcarbonyl)rapamycin, 42-O-(valienoneylcarbonyl)rapamycin, 42-O-[2-(D-valienoneylcarbonyloxy)ethyl]rapamycin, 31-O-(valienoneylcarbonyl)rapamycin, 42-O-(2-hydroxyethyl)-31-O-(valienoneylcarbonyl)rapamycin, PI-103 (3-[4-(4-morpholinyl)pyrido[3′,2′:4,5]furo[3,2-d]pyrimidin-2-yl]-phenol), KU-0063794 ((5-(2-((2R,6S)-2,6-dimethylmorpholino)-4-morpholinopyrido[2,3-d]pyrimidin-7-yl)-2-methoxyphenyl)methanol), PF-04691502 (2-amino-8-((1r,4r)-4-(2-hydroxyethoxy)cyclohexyl)-6-(6-methoxypyridin-3-yl)-4-methylpyrido[2,3-d]pyrimidin-7(8H)-one), CH132799, RG7422 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one), Palomid 529 (3-(4-methoxybenzyloxy)-8-(1-hydroxyethyl)-2-methoxy-6H-benzo[c]chromen-6-one), PP242 (2-(4-amino-1-isopropyl-1H-pyrazolo[3,4-d]pyrimidin-3-yl)-1H-indol-5-ol), XL765 (N-[4-[[[3-[(3,5-dimethoxyphenyl)amino]-2-quinoxalinyl]amino]sulfonyl]phenyl]-3-methoxy-4-methyl-benzamide), GSK1059615 ((Z)-5-((4-(pyridin-4-yl)quinolin-6-yl)methylene)thiazolidine-2,4-dione); PKI-587 (1-(4-(4-(dimethylamino)piperidine-1-carbonyl)phenyl)-3-(4-(4,6-dimorpholino-1,3,5-triazin-2-yl)phenyl)urea); WAY-600 (6-(1H-indol-5-yl)-4-morpholino-1-(1-(pyridin-3-ylmethyl)piperidin-4-yl)-1H-pyrazolo[3,4-d]pyrimidine); WYE-687 (methyl 4-(4-morpholino-1-(1-(pyridin-3-ylmethyl)piperidin-4-yl)-1H-pyrazolo[3,4-d]pyrimidin-6-yl)phenylcarbamate); WYE-125132 (N-[4-[1-(1,4-dioxaspiro[4.5]dec-8-yl)-4-(8-oxa-3-azabicyclo[3.2.1]oct-3-yl)-1H-pyrazolo[3,4-d]pyrimidin-6-yl]phenyl]-N-methyl-urea); and WYE-354 (4-[6-[4-[(methoxycarbonyl)amino]phenyl]-4-(4-morpholinyl)-1H-pyrazolo[3,4-d]pyrimidin-1-yl]-1-piperidinecarboxylic acid methyl ester);
(i) an Akt inhibitor, wherein the Akt inhibitor is selected from the group consisting of: triciribine; RX-0201 (a 20-mer oligonucleotide); perifosine; PX-316 ((R)-2-methoxy-3-(octadecyloxy)propyl ((1R,2R,3S,4R,6R)-2,3,4,6-tetrahydroxycyclohexyl) hydrogen phosphate); API-1 (4-amino-5,8-dihydro-5-oxo-8-P-D-ribofuranosyl-pyrido[2,3-d]pyrimidine-6-carboxamide); SR13668 (diethyl 6-methoxy-5,7-dihydroindolo[2,3-b]carbazole-2,10-dicarboxylate); AZD5363 (4-amino-N-[(1S)-1-(4-chlorophenyl)-3-hydroxypropyl]-1-(7H-pyrrolo[2,3-d]pyrimidin-4-yl)-4-piperidinecarboxamide); miltefosine; miltefosine; GSK690693 (4-(2-(4-amino-1,2,5-oxadiazol-3-yl)-1-ethyl-74(S)-piperidin-3-ylmethoxy)-1H-imidazo[4,5-c]pyridin-4-yl)-2-methylbut-3-yn-2-ol); A-443654 ((2S)-1-(1H-indol-3-yl)-3-[5-(3-methyl-2H-indazol-5-yl)pyridin-3-yl]oxypropan-2-amine); and SR13668 (diethyl 6-methoxy-5,7-dihydroindolo[2,3-b]carbazole-2,10-dicarboxylate);
(j) a Notch inhibitor, wherein the Notch inhibitor is a gamma secretase inhibitor selected from the group consisting of gamma secretase inhibitor I, gamma secretase inhibitor II, gamma secretase inhibitor III, gamma secretase inhibitor IV, gamma secretase inhibitor V, gamma secretase inhibitor VI, gamma secretase inhibitor VII, gamma secretase inhibitor IX, gamma secretase inhibitor X, gamma secretase inhibitor XI, gamma secretase inhibitor XII, gamma secretase inhibitor XIII, gamma secretase inhibitor XIV, gamma secretase inhibitor XVI, gamma secretase inhibitor XVII, gamma secretase inhibitor XIX, gamma secretase inhibitor XX, gamma secretase inhibitor XXI, gamma40 secretase inhibitor I, gamma40 secretase inhibitor II, and isovaleryl-V—V-Sta-A-Sta-OCH₃;
(k) an Hsp90 inhibitor, wherein the Hsp90 inhibitor is selected from the group consisting of: IPI-493 (17-amino-17-demethoxygeldanamycin); IPI-504 (retaspimycin hydrochloride); 17-demethoxy-17-(2-propylamino)-geldanamycin; AUY-922 (5-(2,4-dihydroxy-5-isopropylphenyl)-N-ethyl-4-(4-(morpholinomethyl)phenyl)isoxazole-3-carboxamide); elesclomol; alvespimycin (17-demethoxy-17-[[2-(dimethylamino)ethyl]amino]-geldanamycin hydrochloride); 5′-O-[(4-cyanophenyl)methyl]-8-[[(3,4-dichlorophenyl)methyl]amino]-adenosine; N1-[(3-endo)-8-[5-(cyclopropylcarbonyl)-2-pyridinyl]-8-azabicyclo[3.2.1]oct-3-yl]-2-methyl-5-[[(1R)-1-methylpropyl]amino]-1,4-benzenedicarboxamide; (2,4-dihydroxy-5-isopropylphenyl)(5-(4-methylpiperazin-1-yl)methyl)isoindolin-2-yl)methanone; 4-(6,6-dimethyl-4-oxo-3-(trifluoromethyl)-4,5,6,7-tetrahydroindazol-1-yl)-2-((1r,4r)-4-hydroxycyclohexylamino)benzamide; (1r,4r)-4-(2-carbamoyl-5-(6,6-dimethyl-4-oxo-3-(trifluoromethyl)-4,5,6,7-tetrahydroindazol-1-yl)phenylamino)cyclohexyl 2-aminoacetate; 2-amino-4-(2,4-dichloro-5-(2-(pyrrolidin-1-yl)ethoxy)phenyl)-N-ethylthieno[2,3-d]pyrimidine-6-carboxamide; 6-chloro-9-(4-methoxy-3,5-dimethylpyridin-2-yl)methyl)-9H-purin-2-amine; MPC-3100 ((S)-1-(4-(2-(6-amino-8-((6-bromobenzo[d][1,3]dioxol-5-yl)thio)-9H-purin-9-yl)ethyl)piperidin-1-yl)-2-hydroxypropan-1-one); CCT-018159 (4-[4-(2,3-dihydro-1,4-benzodioxin-6-yl)-5-methyl-1H-pyrazol-3-yl]-6-ethyl-1,3-benzenediol); CCT-129397 (3-(5-chloro-2,4-dihydroxyphenyl)-N-ethyl-4-(4-methoxyphenyl)-1H-pyrazole-5-carboxamide); PU-H71 (6-amino-8-[(6-iodo-1,3-benzodioxol-5-yl)thio]-N-(1-methylethyl)-9H-purine-9-propanamine); SNX-2112 (4-(6,6-dimethyl-4-oxo-3-(trifluoromethyl)-4,5,6,7-tetrahydroindazol-1-yl)-2-((1r,4r)-4-hydroxycyclohexylamino)benzamide; ganetespib; onalespib; XL-888 (2-[[(2R)-butan-2-yl]amino]-4-N-[8-[5-(cyclopropanecarbonyl)pyridin-2-yl]-8-azabicyclo[3.2.1]octan-3-yl]-5-methylbenzene-1,4-dicarboxamide); CU-0305; tanespimycin; macbecin L macbecin II; an 11-O-methyl derivative of geldanamycin; 17-allylamino-17-demethoxygeldanamycin, 17-(dimethylaminoethylamino)-17-demethoxygeldanamycin; 17-[2-(pyrrolidin-1-yl)ethyl]amino-17-demethoxygeldanamycin; 17-(dimethylaminopropylamino)-17-demethoxygeldanamycin; KF58333 (E isomer); cycloproparadicicol; pochonin D; B-zearalenol; celastrol; gedunin; dacinostat; and romidepsin;
(l) a small-molecule kinase inhibitor, wherein the small-molecule kinase inhibitor is selected from the group consisting of afatinib, axitinib, bosutinib, crizotinib, dasatinib, erlotinib, fostamatinib, gefitinib, ibrutinib, lapatinib, lenvatinib, mubritinib, nilotinib, pazopanib, ruxolitinib, sorafenib, sunitinib, SU6656 ((3Z)—N,N-dimethyl-2-oxo-3-(4,5,6,7-tetrahydro-1H-indol-2-ylmethylidene)-2,3-dihydro-1H-indole-5-sulfonamide)), tofacitinib, vandetanib, and vemurafenib,
(m) a monoclonal antibody kinase inhibitor, wherein the monoclonal antibody kinase inhibitor is selected from the group consisting of bevacizumab, cetuximab, panitumumab, ranibizumab, and trastuzumab,
(n) an RNA aptamer kinase inhibitor, wherein the RNA aptamer kinase inhibitor is pegaptanib, and
(o) a pyrimidine analog antimetabolite, wherein the pyrimidine analog antimetabolite is selected from the group consisting of cytarabine, 5-azacytidine, gemcitabine, floxuridine, 5-fluorouracil, capecitabine, 6-azauracil, troxacitabine, thiarabine, sapacitabine, CNDAC, 2′-deoxy-2′-methylidenecytidine, 2′-deoxy-2′-fluoromethylidenecytidine, 2′-deoxy-2′-methylidene-5-fluorocytidine, 2′-deoxy-2′,2′-difluorocytidine, and 2′-C-cyano-2′-deoxy-P-arabinofuranosylcytosine.

119.-137. (canceled)
